SEQUENCE LISTING (107 sequences)

SEQ ID NO: 1 - protein, 84 aa, Homo sapiens
MVATGSLSSKNPASISELLDCGYHPESLLSGECPRCPPFSPPKVGASGSIGWAGFRLPPLPLCRRCHKIHLRRLLSKFSNIFSP

SEQ ID NO: 2 - protein, 30 aa, synthetic construct
  REGION 1..30: note = Amino acids encoded by the first exon
MVATGSLSSKNPASISELLDCGYHPESLLS

SEQ ID NO: 3 - protein, 54 aa, synthetic construct
  REGION 1..54: note = Amino acids encoded by retained introns
GECPRCPPFSPPKVGASGSIGWAGFRLPPLPLCRRCHKIHLRRLLSKFSNIFSP

SEQ ID NO: 4 - protein, 27 aa, synthetic construct
  REGION 1..27: note = IR-1
GECPRCPPFSPPKVGASGSIGWAGFRL

SEQ ID NO: 5 - protein, 27 aa, synthetic construct
  REGION 1..27: note = IR-2
PPLPLCRRCHKIHLRRLLSKFSNIFSP

SEQ ID NO: 6 - DNA, 20 bp, synthetic construct
  misc_feature 1..20: note = forward primer
  source 1..20: mol_type = other DNA
tgctggactgtggctatcac

SEQ ID NO: 7 - DNA, 21 bp, synthetic construct
  misc_feature 1..21: note = reverse primer
  source 1..21: mol_type = other DNA
acaaggacctttgagcaacca

SEQ ID NO: 8 - DNA, 255 bp, synthetic construct
  misc_feature 1..255: note = DIR open reading frame
  source 1..255: mol_type = other DNA
atggttgcaactggcagtttgagcagcaagaacccggccagcatttcagaattgctggactgtggctatcacccagagagcctgctaagtggtgagtgtccccgctgtccacccttctcccctcccaaggtcggtgcctctggatctattgggtgggcgggctttcgacttcctcccctacctctatgtaggaggtgccacaaaatccatctgaggagactcctttccaaattttctaacatcttctctccatga

SEQ ID NO: 9 - DNA, 2346 bp, synthetic construct
  misc_feature 1..2346: note = Gelsolin open reading frame
  source 1..2346: mol_type = other DNA
atggctccgcaccgccccgcgcccgcgctgctttgcgcgctgtccctggcgctgtgcgcgctgtcgctgcccgtccgcgcggccactgcgtcgcggggggcgtcccaggcgggggcgccccaggggcgggtgcccgaggcgcggcccaacagcatggtggtggaacaccccgagttcctcaaggcagggaaggagcctggcctgcagatctggcgtgtggagaagttcgatctggtgcccgtgcccaccaacctttatggagacttcttcacgggcgacgcctacgtcatcctgaagacagtgcagctgaggaacggaaatctgcagtatgacctccactactggctgggcaatgagtgcagccaggatgagagcggggcggccgccatctttaccgtgcagctggatgactacctgaacggccgggccgtgcagcaccgtgaggtccagggcttcgagtcggccaccttcctaggctacttcaagtctggcctgaagtacaagaaaggaggtgtggcatcaggattcaagcacgtggtacccaacgaggtggtggtgcagagactcttccaggtcaaagggcggcgtgtggtccgtgccaccgaggtacctgtgtcctgggagagcttcaacaatggcgactgcttcatcctggacctgggcaacaacatccaccagtggtgtggttccaacagcaatcggtatgaaagactgaaggccacacaggtgtccaagggcatccgggacaacgagcggagtggccgggcccgagtgcacgtgtctgaggagggcactgagcccgaggcgatgctccaggtgctgggccccaagccggctctgcctgcaggtaccgaggacaccgccaaggaggatgcggccaaccgcaagctggccaagctctacaaggtctccaatggtgcagggaccatgtccgtctccctcgtggctgatgagaaccccttcgcccagggggccctgaagtcagaggactgcttcatcctggaccacggcaaagatgggaaaatctttgtctggaaaggcaagcaggcaaacacggaggagaggaaggctgccctcaaaacagcctctgacttcatcaccaagatggactaccccaagcagactcaggtctcggtccttcctgagggcggtgagaccccactgttcaagcagttcttcaagaactggcgggacccagaccagacagatggcctgggcttgtcctacctttccagccatatcgccaacgtggagcgggtgcccttcgacgccgccaccctgcacacctccactgccatggccgcccagcacggcatggatgacgatggcacaggccagaaacagatctggagaatcgaaggttccaacaaggtgcccgtggaccctgccacatatggacagttctatggaggcgacagctacatcattctgtacaactaccgccatggtggccgccaggggcagataatctataactggcagggtgcccagtctacccaggatgaggtcgctgcatctgccatcctgactgctcagctggatgaggagctgggaggtacccctgtccagagccgtgtggtccaaggcaaggagcccgcccacctcatgagcctgtttggtgggaagcccatgatcatctacaagggcggcacctcccgcgagggcgggcagacagcccctgccagcacccgcctcttccaggtccgcgccaacagcgctggagccacccgggctgttgaggtattgcctaaggctggtgcactgaactccaacgatgcctttgttctgaaaaccccctcagccgcctacctgtgggtgggtacaggagccagcgaggcagagaagacgggggcccaggagctgctcagggtgctgcgggcccaacctgtgcaggtggcagaaggcagcgagccagatggcttctgggaggccctgggcgggaaggctgcctaccgcacatccccacggctgaaggacaagaagatggatgcccatcctcctcgcctctttgcctgctccaacaagattggacgttttgtgatcgaagaggttcctggtgagctcatgcaggaagacctggcaacggatgacgtcatgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcc

SEQ ID NO: 10 - protein, 8 aa, synthetic construct
  REGION 1..8: note = A2D10 HCDR1
GYTFTEYT

SEQ ID NO: 11 - protein, 8 aa, synthetic construct
  REGION 1..8: note = A2D10 HCDR2
IYPHNGGT

SEQ ID NO: 12 - protein, 8 aa, synthetic construct
  REGION 1..8: note = A2D10 HCDR3
TEDLTFDY

SEQ ID NO: 13 - protein, 115 aa, synthetic construct
  REGION 1..115: note = A2D10
EVQLQQSGPELVKPGASVKISCKTSGYTFTEYTMHWVKQSHGKSLEWIGGIYPHNGGTNYNQKFKGKATLTVDKSSSTAYMELRSLTSEDSAVYYCTEDLTFDYWGQGTTLTVSS

SEQ ID NO: 14 - protein, 345 aa, synthetic construct
  REGION 1..345: note = A2D10
GAGGTCCAGCTGCAACAGTCTGGACCTGAGCTGGTGAAGCCTGGGGCTTCAGTGAAGATATCCTGCAAGACTTCTGGATACACATTCACTGAATACACCATGCACTGGGTGAAGCAGAGCCATGGAAAGAGCCTTGAGTGGATTGGAGGTATTTATCCTCACAATGGTGGTACTAACTACAACCAGAAGTTCAAGGGCAAGGCCACATTGACTGTAGACAAGTCCTCCAGCACAGCCTACATGGAGCTCCGCAGCCTGACATCTGAGGATTCTGCAGTCTATTACTGTACAGAAGATCTAACTTTTGACTACTGGGGCCAAGGCACCACTCTCACAGTCTCCTCA

SEQ ID NO: 15 - protein, 6 aa, synthetic construct
  REGION 1..6: note = A2D10 LCDR1
QDISNY

SEQ ID NO: 16 - skipped

SEQ ID NO: 17 - protein, 9 aa, synthetic construct
  REGION 1..9: note = A2D10 LCDR3
LQYSKLPFT

SEQ ID NO: 18 - protein, 107 aa, synthetic construct
  REGION 1..107: note = A2D10
DIQMTQTTSSLSASLGDRVTISCSASQDISNYLNWYQQKPDGTVKLLIYYTSSLHSGVPSRFSGSGSGTDYSLTISNLEPEDIATYHCLQYSKLPFTFGSGTKLEIK

SEQ ID NO: 19 - protein, 321 aa, synthetic construct
  REGION 1..321: note = A2D10
GATATCCAGATGACACAGACTACATCCTCCCTGTCTGCCTCTCTGGGAGACAGAGTCACCATCAGTTGCAGTGCAAGTCAGGACATTAGCAATTATTTAAACTGGTATCAGCAGAAACCAGATGGAACTGTTAAACTCCTGATCTATTACACATCAAGTTTACACTCAGGAGTCCCATCAAGGTTCAGTGGCAGTGGGTCTGGGACAGATTATTCTCTCACCATCAGCAACCTGGAACCTGAAGATATTGCCACTTACCATTGTCTACAGTATAGTAAGCTTCCATTCACGTTCGGCTCGGGGACAAAGTTGGAAATAAAA

SEQ ID NO: 20 - protein, 8 aa, synthetic construct
  REGION 1..8: note = HCDR1
GYTFSSYW

SEQ ID NO: 21 - protein, 8 aa, synthetic construct
  REGION 1..8: note = HCDR2
ILPGSGTT

SEQ ID NO: 22 - protein, 12 aa, synthetic construct
  REGION 1..12: note = HCDR3
ARSYDGYYVFDY

SEQ ID NO: 23 - protein, 119 aa, synthetic construct
  REGION 1..119: note = VH
QVQLKESGAELMKPGASVKISCKATGYTFSSYWIEWVKQRPGHGLEWIGEILPGSGTTNYNDKFKGKATFTADTSSNTAYMQLSNLTSEDSAVYYCARSYDGYYVFDYWGQGTTLTVSS

SEQ ID NO: 24 - protein, 357 aa, synthetic construct
  REGION 1..357: note = nucleotides encoding VH
CAGGTGCAGCTGAAGGAGTCAGGAGCTGAGCTGATGAAGCCTGGGGCCTCAGTGAAGATATCCTGCAAGGCTACTGGCTACACATTCAGTAGCTACTGGATAGAGTGGGTAAAGCAGAGGCCTGGACATGGCCTTGAGTGGATTGGAGAGATTTTACCTGGAAGTGGTACTACTAACTACAATGACAAGTTCAAGGGCAAGGCCACATTCACTGCAGATACATCCTCCAACACAGCCTACATGCAACTCAGCAACCTGACATCTGAGGACTCTGCCGTCTATTACTGTGCAAGATCCTATGATGGTTACTACGTCTTTGACTACTGGGGCCAAGGCACCACTCTCACAGTCTCCTCA

SEQ ID NO: 25 - protein, 10 aa, synthetic construct
  REGION 1..10: note = LCDR1
QSVDYDGDSY

SEQ ID NO: 26 - skipped

SEQ ID NO: 27 - protein, 9 aa, synthetic construct
  REGION 1..9: note = LCDR3
QQSNEDPRT

SEQ ID NO: 28 - protein, 111 aa, synthetic construct
  REGION 1..111: note = VL
DIELTQSPASLAVSLGQRATISCKASQSVDYDGDSYMNWYQQKPGQPPKLLIYAASNLESGIPARFSGSGSGTDFTLNIHPVEEEDAATYYCQQSNEDPRTFGGGTKLEIK

SEQ ID NO: 29 - protein, 333 aa, synthetic construct
  REGION 1..333: note = nucleotides encoding VL
GACATTGAGCTCACCCAATCTCCAGCTTCTTTGGCTGTGTCTCTAGGGCAGAGGGCCACCATCTCCTGCAAGGCCAGCCAAAGTGTTGATTATGATGGTGATAGTTATATGAACTGGTACCAACAGAAACCAGGACAGCCACCCAAACTCCTCATCTATGCTGCATCCAATCTAGAATCTGGGATCCCAGCCAGGTTTAGTGGCAGTGGGTCTGGGACAGACTTCACCCTCAACATCCATCCTGTGGAGGAGGAGGATGCTGCAACCTATTACTGTCAGCAAAGTAATGAGGATCCTCGGACGTTCGGTGGAGGCACCAAGCTGGAAATCAAA

SEQ ID NO: 30 - protein, 17 aa, synthetic construct
  REGION 1..17: note = HCDR3
ARSGVITARGDYYAMDY

SEQ ID NO: 31 - protein, 124 aa, synthetic construct
  REGION 1..124: note = VH
EVQLQQSGAELMKPGASVKISCKATGYTFSSYWIEWVKQRPGHGLEWIGEILPGSGTTNYNEKFKGKATFTVDTSSNTAYMQLSSLTSEDSAVYYCARSGVITARGDYYAMDYWGQGTSVTVSS

SEQ ID NO: 32 - protein, 372 aa, synthetic construct
  REGION 1..372: note = nucleotides encoding VH
GAGGTCCAGCTGCAGCAGTCAGGAGCTGAGCTGATGAAGCCTGGGGCCTCAGTGAAGATATCCTGCAAGGCTACTGGCTACACATTCAGTAGCTACTGGATAGAGTGGGTAAAGCAGAGGCCTGGACATGGCCTTGAGTGGATTGGAGAAATTTTACCTGGAAGTGGTACTACTAACTACAATGAGAAGTTCAAGGGCAAGGCCACATTCACTGTAGATACATCCTCCAACACAGCCTACATGCAACTCAGCAGCCTGACATCTGAGGACTCTGCCGTCTATTATTGTGCAAGATCAGGAGTGATTACGGCGAGAGGGGATTACTATGCTATGGACTACTGGGGTCAAGGAACCTCAGTCACCGTCTCCTCA

SEQ ID NO: 33 - protein, 111 aa, synthetic construct
  REGION 1..111: note = VL
DIVMTQSPASLAVSLGQRATISCKASQSVDYDGDSYMNWYQQKPGQPPKLLIYAASNLDSGIPARFSGSGSGTDFTLNIHPVEEEDAATYYCQQSNEDPRTFGGGTKLEIK

SEQ ID NO: 34 - protein, 333 aa, synthetic construct
  REGION 1..333: note = nucleotides encoding VL
GACATTGTAATGACCCAATCTCCAGCTTCTTTGGCTGTGTCTCTAGGGCAGAGGGCCACCATATCCTGCAAGGCCAGCCAAAGTGTTGATTATGATGGTGATAGTTATATGAACTGGTACCAACAGAAACCAGGACAGCCACCCAAACTCCTCATCTATGCTGCATCCAATCTAGATTCTGGGATCCCAGCCAGGTTTAGTGGCAGTGGGTCTGGGACAGACTTCACCCTCAACATCCATCCTGTGGAGGAGGAGGATGCTGCAACCTATTACTGTCAGCAAAGTAATGAGGATCCTCGGACGTTCGGTGGAGGCACCAAGCTGGAAATCAAA

SEQ ID NO: 35 - protein, 8 aa, synthetic construct
  REGION 1..8: note = HCDR1
GYTFSNYW

SEQ ID NO: 36 - protein, 9 aa, synthetic construct
  REGION 1..9: note = HCDR2
ILPGSYYST

SEQ ID NO: 37 - protein, 17 aa, synthetic construct
  REGION 1..17: note = HCDR3
ARSGMITTRGDYFAMDY

SEQ ID NO: 38 - protein, 125 aa, synthetic construct
  REGION 1..125: note = VH
EVQLQQSGAELMKPGASVKISCKATGYTFSNYWIEWIKQRPGHGLEWIGEILPGSYYSTNYNEKFKGKATFTADTSSNTAYIQLSSLTSEDSAVYYCARSGMITTRGDYFAMDYWGQGTSVTVSS

SEQ ID NO: 39 - protein, 375 aa, synthetic construct
  REGION 1..375: note = nucleotide encoding VH
GAGGTCCAGCTGCAGCAGTCAGGAGCTGAGCTGATGAAGCCTGGGGCCTCAGTGAAGATATCCTGCAAGGCTACTGGCTACACATTCAGTAACTACTGGATAGAGTGGATAAAGCAGAGGCCTGGACATGGCCTTGAATGGATTGGAGAGATTTTACCTGGAAGTTATTATAGTACTAACTACAATGAGAAGTTCAAGGGCAAGGCCACATTCACTGCAGATACTTCCTCCAACACAGCCTACATACAACTCAGCAGCCTGACATCTGAGGACTCTGCCGTCTATTACTGTGCAAGATCAGGAATGATTACGACGAGAGGGGATTACTTTGCTATGGACTACTGGGGTCAAGGAACCTCAGTCACCGTCTCCTCA

SEQ ID NO: 40 - protein, 111 aa, synthetic construct
  REGION 1..111: note = VL
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPRTFGGGTKLEIK

SEQ ID NO: 41 - protein, 333 aa, synthetic construct
  REGION 1..333: note = nucleotide encoding VL
GACATTGTGCTCACCCAGTCTCCAGCTTCTTTGGCTGTGTCTCTAGGGCAGAGGGCCACCATCTCCTGCAAGGCCAGCCAAAGTGTTGATTATGATGGTGATAGTTATATGAACTGGTACCAACAGAAACCAGGACAGCCACCCAAACTCCTCATCTATGCTGCATCCAATCTAGAATCTGGGATCCCAGCCAGGTTTAGTGGCAGTGGGTCTGGGACAGACTTCACCCTCAACATCCATCCTGTGGAGGAGGAGGATGCTGCAACCTATTACTGTCAGCAAAGTAATGAGGATCCTCGGACGTTCGGTGGAGGCACCAAGCTGGAAATCAAA

SEQ ID NO: 42 - protein, 8 aa, synthetic construct
  REGION 1..8: note = HCDR2
ILPGSGST

SEQ ID NO: 43 - protein, 15 aa, synthetic construct
  REGION 1..15: note = HCDR3
ARTDDGYLSYFAMDY

SEQ ID NO: 44 - protein, 122 aa, synthetic construct
  REGION 1..122: note = VH
QVQLQQSGAELMKPGASVKISCKATGYTFSSYWIEWVKQRPGHGLEWIGEILPGSGSTNYNEKFKGKATFTADTSSNTAYMQLNSLTSEDSAVYYCARTDDGYLSYFAMDYWGQGTSVTVSS

SEQ ID NO: 45 - protein, 366 aa, synthetic construct
  REGION 1..366: note = nucleotide encoding VL
CAGGTTCAGCTGCAGCAGTCTGGAGCTGAGCTGATGAAGCCTGGGGCCTCAGTGAAGATATCCTGCAAGGCTACTGGCTACACATTCAGTAGCTACTGGATAGAGTGGGTTAAGCAGAGGCCTGGACATGGCCTTGAGTGGATTGGGGAGATTTTACCTGGAAGTGGTAGTACTAACTACAATGAAAAGTTCAAGGGCAAGGCCACATTCACTGCAGATACATCCTCCAACACAGCCTACATGCAACTCAACAGCCTGACATCTGAGGACTCTGCCGTCTATTACTGTGCGAGAACCGATGATGGTTACCTATCTTACTTTGCTATGGACTACTGGGGTCAAGGAACCTCAGTCACCGTCTCCTCA

SEQ ID NO: 46 - protein, 111 aa, synthetic construct
  REGION 1..111: note = VL
DILMTQTPASLAVSLGQRATISCKASQSVDYDGDSYMNWYQQKPGQPPKLLIYAASNLESGIPARFSGSGSGTDFTLNIHPVEEEDAATYYCQQSNEDPRTFGGGTKLEIK

SEQ ID NO: 47 - protein, 333 aa, synthetic construct
  REGION 1..333: note = nucleotide encoding VL
GACATTTTGATGACCCAGACTCCAGCTTCTTTGGCTGTGTCTCTAGGGCAGAGGGCCACCATCTCCTGCAAGGCCAGCCAAAGTGTTGATTATGATGGTGATAGTTATATGAACTGGTACCAACAGAAACCAGGACAGCCACCCAAACTCCTCATCTATGCTGCATCCAATCTAGAATCTGGGATCCCAGCCAGGTTTAGTGGCAGTGGGTCTGGGACAGACTTCACCCTCAACATCCATCCTGTGGAGGAGGAGGATGCTGCAACCTATTACTGTCAGCAAAGTAATGAGGATCCTCGGACGTTCGGTGGCGGCACCAAGCTGGAAATCAAA

SEQ ID NO: 48 - protein, 8 aa, synthetic construct
  REGION 1..8: note = HCDR2
ILPGGYNT

SEQ ID NO: 49 - protein, 17 aa, synthetic construct
  REGION 1..17: note = HCDR3
ARSGMITTTGDYYAMDY

SEQ ID NO: 50 - protein, 124 aa, synthetic construct
  REGION 1..124: note = VH
QVQLQQSGAEVMKPGASVKISCKATGYTFSSYWIEWIKQRPGHGLEWIGEILPGGYNTNSNENFKGKATFTADTSSNTAYMQLSSLTSEDSAVYYCARSGMITTTGDYYAMDYWGQGTSVTVSS

SEQ ID NO: 51 - protein, 372 aa, synthetic construct
  REGION 1..372: note = nucleotide encoding VH
CAGGTGCAGCTGCAGCAGTCTGGAGCTGAGGTGATGAAGCCAGGGGCCTCAGTGAAGATATCCTGCAAGGCTACTGGCTACACATTCAGTAGCTACTGGATAGAGTGGATAAAGCAGAGGCCTGGACATGGCCTTGAGTGGATTGGAGAGATTTTACCTGGAGGCTATAATACTAACTCCAATGAGAACTTCAAGGGCAAGGCCACATTCACTGCAGATACATCCTCCAACACAGCCTACATGCAACTCAGCAGCCTGACATCTGAGGACTCTGCCGTCTATTACTGTGCAAGATCAGGAATGATTACGACGACAGGGGATTACTATGCTATGGACTACTGGGGTCAAGGAACCTCAGTCACCGTCTCCTCA

SEQ ID NO: 52 - protein, 10 aa, synthetic construct
  REGION 1..10: note = LCDR1
QSVDYDGESY

SEQ ID NO: 53 - protein, 9 aa, synthetic construct
  REGION 1..9: note = LCDR3
QQSNDDPRT

SEQ ID NO: 54 - protein, 111 aa, synthetic construct
  REGION 1..111: note = VL
DIELTQSPASLAVSLGQRATISCKASQSVDYDGESYMNWYQQKPGQPPKLLIYAASNLESGIPARFSGSGSGTDFTLNIHPVEEEDAATYYCQQSNDDPRTFGGGTKLEIK

SEQ ID NO: 55 - protein, 333 aa, synthetic construct
  REGION 1..333: note = nucleotide encoding VL
GACATTGAGCTGACCCAATCTCCAGCTTCTTTGGCTGTGTCTCTAGGCCAGAGGGCCACCATCTCCTGCAAGGCCAGCCAAAGTGTTGATTATGATGGTGAGAGTTATATGAACTGGTACCAACAGAAACCAGGACAGCCACCCAAACTCCTCATCTATGCTGCATCCAATTTAGAGTCTGGGATCCCAGCCAGGTTTAGTGGCAGTGGGTCTGGGACAGACTTCACCCTCAACATCCATCCTGTGGAGGAGGAGGATGCTGCAACCTATTACTGTCAGCAAAGTAATGACGATCCTCGGACGTTCGGTGGAGGCACCAAGCTGGAAATCAAA

SEQ ID NO: 56 - protein, 8 aa, synthetic construct
  REGION 1..8: note = HCDR1
GYTFSRYW

SEQ ID NO: 57 - protein, 17 aa, synthetic construct
  REGION 1..17: note = HCDR3
ARSGVITTRGDYFAMDY

SEQ ID NO: 58 - protein, 125 aa, synthetic construct
  REGION 1..125: note = VH
QVQLQQSGAELMKPGASVKISCKATGYTFSRYWIEWIKQRPGHGLEWIGEILPGSYYSTNSNEKFKGKATFTADTSSNTAYMQLSSLTSEDSAVYYCARSGVITTRGDYFAMDYWGHGTSVTVSS

SEQ ID NO: 59 - protein, 375 aa, synthetic construct
  REGION 1..375: note = nucleotide encoding VL
CAGGTTCAACTGCAGCAGTCTGGAGCTGAGCTGATGAAGCCTGGGGCCTCAGTGAAGATATCCTGCAAGGCTACTGGCTACACATTCAGTAGGTACTGGATAGAGTGGATAAAGCAGAGGCCTGGACATGGCCTTGAGTGGATTGGAGAGATTTTACCTGGAAGTTATTATAGTACTAACTCCAATGAGAAGTTCAAGGGCAAGGCCACATTCACTGCAGATACATCCTCCAACACAGCCTACATGCAACTCAGCAGCCTGACATCTGAGGACTCTGCCGTCTACTACTGTGCAAGATCAGGAGTAATTACGACGAGAGGGGATTACTTTGCTATGGACTACTGGGGTCACGGAACCTCAGTCACCGTCTCCTCA

SEQ ID NO: 60 - protein, 111 aa, synthetic construct
  REGION 1..111: note = VL
DIVLTQSPASLAVSLGQRATISCKASQSVDYDGDSYMNWYQQKPGQPPKLLMYAASNLESGIPARFSGSGSGTDFTLNIHPVEEEDAATYYCQQSNEDPRTFGGGTKLEIK

SEQ ID NO: 61 - protein, 333 aa, synthetic construct
  REGION 1..333: note = nucleotide encoding VL
GACATTGTGCTCACCCAGTCTCCAGCTTCTTTGGCTGTGTCTCTAGGGCAGAGGGCCACCATCTCCTGCAAGGCCAGCCAAAGTGTTGATTATGATGGTGATAGTTATATGAACTGGTACCAACAGAAACCAGGACAGCCACCCAAACTCCTCATGTATGCTGCATCCAATCTAGAATCTGGGATCCCAGCCAGGTTTAGTGGCAGTGGGTCTGGGACAGACTTCACCCTCAACATCCATCCTGTGGAGGAGGAGGATGCTGCTACCTATTACTGTCAGCAAAGTAATGAGGATCCTCGGACGTTCGGTGGAGGCACCAAGCTGGAAATCAAA

SEQ ID NO: 62 - protein, 17 aa, synthetic construct
  REGION 1..17: note = HCDR3
ARSGIITTRGDYFAMDY

SEQ ID NO: 63 - protein, 125 aa, synthetic construct
  REGION 1..125: note = VH
QVQLQQSGAELMKPGASVKISCKATGYTFSNYWIEWIKQRPGHGLEWIGEILPGSYYSTNYNEKFKDKATFTADTSSNTAYIQLSSLTSEDSAVYYCARSGIITTRGDYFAMDYWGQGTSVTVSS

SEQ ID NO: 64 - protein, 375 aa, synthetic construct
  REGION 1..375: note = nucleotide encoding VH
CAGGTTCAGCTGCAGCAGTCTGGAGCTGAGCTGATGAAGCCTGGGGCCTCAGTGAAGATATCCTGCAAGGCTACTGGCTACACATTCAGTAACTACTGGATAGAGTGGATAAAGCAGAGGCCTGGACATGGCCTTGAGTGGATTGGAGAGATTTTACCTGGAAGTTATTATAGTACTAACTACAATGAGAAGTTCAAGGACAAGGCCACATTCACTGCAGATACATCCTCCAACACAGCCTACATACAACTCAGCAGCCTGACATCTGAGGACTCTGCCGTCTATTATTGTGCAAGATCAGGAATAATTACGACGAGAGGGGATTACTTTGCTATGGACTACTGGGGTCAAGGAACCTCAGTCACTGTCTCCTCA

SEQ ID NO: 65 - protein, 124 aa, synthetic construct
  REGION 1..124: note = VH
QVQLQQSGAELMKPGASVKISCKATGYTFSNYWIEWVKQRPGHGLEWIGEILPGSGSTNYNEKFKGKATFTADTSSNTAYMQLSSLTSEDSAVYYCARSGMITTRGDYFAMDYWGQGTSVTVSS

SEQ ID NO: 66 - protein, 372 aa, synthetic construct
  REGION 1..372: note = nucleotide encoding VH
CAGGTGCAGCTGCAGCAGTCTGGAGCTGAGCTGATGAAGCCTGGGGCCTCAGTGAAGATATCCTGCAAGGCTACTGGCTACACATTCAGTAACTACTGGATAGAGTGGGTAAAGCAGAGGCCTGGACATGGCCTTGAGTGGATTGGAGAGATTTTACCTGGAAGTGGTAGTACTAACTACAATGAGAAGTTCAAGGGCAAGGCCACATTCACTGCAGATACATCCTCCAACACAGCCTACATGCAACTCAGCAGCCTGACATCTGAGGACTCTGCCGTCTATTACTGTGCAAGATCGGGGATGATTACGACGAGAGGGGATTACTTTGCTATGGACTACTGGGGTCAAGGAACCTCAGTCACCGTCTCCTCA

SEQ ID NO: 67 - protein, 9 aa, synthetic construct
  REGION 1..9: note = LCDR3
QQSYDDPRA

SEQ ID NO: 68 - protein, 111 aa, synthetic construct
  REGION 1..111: note = VL
DIELTQSPASLAVSLGQRATISCKASQSVDYDGESYMNWYQQKPGQPPKLLIYAASNLESGIPARFSGSGSGTDFTLNIHPVEEEDAATYYCQQSYDDPRAFGGGTKLEIK

SEQ ID NO: 69 - protein, 333 aa, synthetic construct
  REGION 1..333: note = nucleotide encoding VL
GACATTGAGCTGACCCAATCTCCAGCTTCTTTGGCTGTGTCTCTAGGCCAGAGGGCCACCATCTCCTGCAAGGCCAGCCAAAGTGTTGATTATGATGGTGAGAGTTATATGAACTGGTACCAACAGAAACCAGGACAGCCACCCAAACTCCTCATCTATGCTGCATCCAATCTAGAATCTGGGATCCCAGCCAGGTTTAGTGGCAGTGGGTCTGGGACAGACTTCACCCTCAACATCCATCCTGTGGAGGAGGAGGATGCTGCAACCTATTACTGTCAGCAAAGTTATGACGATCCTCGGGCGTTCGGTGGAGGCACCAAGCTGGAAATCAAA

SEQ ID NO: 70 - protein, 8 aa, synthetic construct
  REGION 1..8: note = HCDR1
  SITE 5: note = misc_feature - Xaa = Ser Thr
  SITE 6: note = misc_feature - Xaa = Glu Asn Arg Ser
  SITE 8: note = misc_feature - Xaa = Thr Trp
  source 1..8: note = HCDR1 general formula
GYTFXXYX

SEQ ID NO: 71-75 - skipped (5 entries)

SEQ ID NO: 76 - DNA, 35 bp, synthetic construct
  misc_feature 1..35: note = pCMV-flag-DIRFlag-DIR
  source 1..35: mol_type = other DNA
ccatggaggcccgaattcggatggttgcaactggc

SEQ ID NO: 77 - DNA, 44 bp, synthetic construct
  misc_feature 1..44: note = pCMV-flag-DIRFlag-DIR
  source 1..44: mol_type = other DNA
actcatcaatgtatcttatcttatggagagaagatgttagaaaa

SEQ ID NO: 78 - DNA, 30 bp, synthetic construct
  misc_feature 1..30: note = pcDNA3.1-DDIT4L-myc-his
  source 1..30: mol_type = other DNA
gccaccatggttgcaactggcagtttgagc

SEQ ID NO: 79 - DNA, 30 bp, synthetic construct
  misc_feature 1..30: note = pcDNA3.1-DDIT4L-myc-his
  source 1..30: mol_type = other DNA
gaattccaccacactggactagtggatccg

SEQ ID NO: 80 - DNA, 32 bp, synthetic construct
  misc_feature 1..32: note = Gelsolin-pEGFP
  source 1..32: mol_type = other DNA
tcaagcttcgaattcatggctccgcaccgccc

SEQ ID NO: 81 - DNA, 35 bp, synthetic construct
  misc_feature 1..35: note = Gelsolin-pEGFP
  source 1..35: mol_type = other DNA
cgggcccgcggtaccggcagccagctcagccatgg

SEQ ID NO: 82 - DNA, 32 bp, synthetic construct
  misc_feature 1..32: note = pcDNA3.1-Gelsolin-myc-his
  source 1..32: mol_type = other DNA
gtggaattcgccaccatggctccgcaccgccc

SEQ ID NO: 83 - DNA, 35 bp, synthetic construct
  misc_feature 1..35: note = pcDNA3.1-Gelsolin-myc-his
  source 1..35: mol_type = other DNA
ccctctagactcgagggcagccagctcagccatgg

SEQ ID NO: 84 - RNA, 19 nt, synthetic construct
  misc_feature 1..19: note = DIR siRNA1
  source 1..19: mol_type = other RNA
tttggaaaggagtctcctc

SEQ ID NO: 85 - RNA, 19 nt, synthetic construct
  misc_feature 1..19: note = DIR siRNA1
  source 1..19: mol_type = other RNA
gaggagactcctttccaaa

SEQ ID NO: 86 - RNA, 19 nt, synthetic construct
  misc_feature 1..19: note = DIR siRNA2
  source 1..19: mol_type = other RNA
tgttagaaaatttggaaag

SEQ ID NO: 87 - RNA, 19 nt, synthetic construct
  misc_feature 1..19: note = DIR siRNA2
  source 1..19: mol_type = other RNA
ctttccaaattttctaaca

SEQ ID NO: 88 - DNA, 23 bp, synthetic construct
  source 1..23: mol_type = other DNA
  misc_feature 1..23: note = Knock in QDLIR sgRNA
atttcagagttgttggacggtgg

SEQ ID NO: 89 - DNA, 3000 bp, synthetic construct
  misc_feature 1..3000: note = 5
  source 1..3000: mol_type = other DNA
caacttgactatatttggaatgaactacaatccagaattggaaggctcaccagtgacccttatctggaggcttggagatccttatctggatcttggtatggagatcttgagccatagtgactatggattccagaagattgaatctcagagtttaaggaacacacctttaacctgggctacacctttcatctgggattaaaggtgtggtggaacacacctttaatctgggctacaccttctgctggagacaatataaggacattggaagaagggagtctagctcttgctccttcgcctgcttgctgcgtgagactgagtaactgctagatccttggacttccattcacagcttctactgaacaattgttgggaattgggctgccgactgtaagtcatcaataaattcctttactatccataagttctgtgactctagagaaccctgactaatacagtagtcttggtttttcttgattctgtttgaatccttaaaatatttcattttcttaagtcatctacttagatgcaaagatagaagttggtgagagcatgtggcttgaaatcatgttaggtgacctttaagaaagacaatctctgtcggttactgcaggtatatgctctacacctcctgattcctcctgccctaagcttcctgatagcatcactcgttcagtcaaaaagaatgctcttatttgaaatttttgcatatgggtaaattgccttctgagataaaagcccagagagtgatataaaattgtagctaggtgagccaaaagtaaataccacattctagaactttctataactttgtcccggtggaagtatgaattatgcctaaatacccgagtggaaatgctgcattttctctattctacccacgtccattctgatcacttactatcataacactaataacattccttgaagcagggaggacgcagctgtcattaacaaggctgactagataaggaccagcggctctgtagcagctaaaggacccattctaaccggcttaaatgggattgcttgttttccacttttgttattaaaatagaagtggaaatctgctttccccagcctatcagcatcccgtgcctgagcctgcctgaccttgagcagtgcagctgcttcgggagagttaaaaatggaccttcaagctggctttagtttctgttgctctgcctgcaagtgtcctcccactgatctgtttatcacactctctttaccacttttgttgttgtttttccttctgagagtttttttagcctggacagcggatcctacttctcaaggccctaacctgcaggtccttccttgcacgtggggacccaaagtgtgcgggagtggtgctggaacctggccttgaatgaggttcaccctcaccccaagtatctcggtctcttccttttctgatccacctcaaccatctcagcaacctcagcaacctcgctgggaacgcccacccattgcaaaccctgtgccactggcgcagggcagcaggaagggtggcttagtaaagttagaagaaaacgtttggcatcgtgcttttagtggtgtcccagaccctctgttccctcttgctcaagactccccggaaagagtcgcagagcttatcctagcaggacaactctttattctcatgccccacaagctttttaaagaccactaaagcttaaacttcttcccaaacaagcccaccgggaacttttgcttctgggaatggcaattgctcctaactatcctgcggctggagcaggcaactgagatcctgttagtggttttgtttagtttcttggcactggccctcttcaattcaagttctgtgcctttcgaagccaggtcttagaacagggccaggctcgccctggccgcggagaaaaacggtttagtcacagatgtagtatccccaaagatgccccattgattcctgagtgcagctgaagctagagagcccttaccacatcccgaccacctgcggggagactgggtgcggactggagctgatgggttagagtcagaacatgggcataaaggatgctcacagagcacagtgtcaactaagccaaatacttattcagaatgacacagcaaagttttgggtcatgcctaacaggacctggagcttctagctgcttgaaaagttccatagccaaaacctgagcagtctgagcctggaagcaggcaagctgtgggcaggggctcacgccaaagaatctaggttagtggccgaagggagatgctctttaggagggtgaccagtggccaactcagggtgaccagtggccaactcaggaagcagcaggtgtccgcccaaggcgaggctgatcaccaactcccaggggcttgcctgggcccctgcctgggaaggaactgaaaggtgacctgaccctgcaggggcgggccggggcacgggcccgagcgagtggcgcaggtgggcgtgccggccaggaggagaggcgggacctataaagcgctctccattcgggaggcggagcggccgggtgcagcctgtgctggtgggcgcggtggcccgcagccttcggcttgctgcaggactgtgcaggggaccactgtccaagcttcggactactgaggggcgcctgcctcggtttacccttcagcgtctggtgaaatccggcaggtgagcgacccgaggaggcgggatagggcagggtcgcttcccagttgtgctgactttccagctacttccagcctgttggctttagttggcgcctccgctcctagggctttgtcaacttgagttgctaaggctggtccctccctttggaagggcagtgcccgggctagttcgccctggcgatgccgggactctgaggagcctgtgcacttcttgcgtgcttttgcaaacttctcactttttcctcccagcgcctagggaaagatccgttctgctccgcgagggaaacagagccgttgaccatggttgcaacgggcagtttgagcagtaagaacccggccagcatttcagagttgttggac

SEQ ID NO: 90 - DNA, 195 bp, synthetic construct
  misc_feature 1..195: note = QDLIR 64
  source 1..195: mol_type = other DNA
tgtggctatcacccagagagcctgctaagtggtgagtgtccccgctgtccacccttctcccctcccaaggtcggtgcctctggatctattgggtgggcgggctttcgacttcctcccctacctctatgtaggaggtgccacaaaatccatctgaggagactcctttccaaattttctaacatcttctctccatga

SEQ ID NO: 91 - DNA, 3000 bp, synthetic construct
  misc_feature 1..3000: note = 3
  source 1..3000: mol_type = other DNA
ggtggctatcaccctgggagtctgctaagtggtgagtctctgggctgtccacctctttcctatccgaaggttgcggggcccctctctagtgtaagcgtccctgcctgagtgcgcgagacttcatctcctcctagagacacagaggactatgaaacccatctgagatctctttctaaacttcctttgcattgtctgcccaccaaaaacaaacgtaaggggaataatgcgttgttccagtctgctaagaggctcttggggatgaagttccctatctcatggttggaacattaagtgtacaggctgggtgtgtttaaagacatgcgcttacttgtgtagctagcaccaccacgtccgtcccccctccccccccccccccaaattctcccttttctgtttgccacctaatccttctctttcccgtctccagaagaccatccttcggcattttgttaccacgttagttttgcttttagaatgttaaataataagtgtatatgctcttctcagttcggtttgctttcccaccgtgtgatgttgtagagttatacacttacctcatcagtgagttccttttcgttgctaagtaagattctattatgaaatgcacccactgtgcattaatgtcagaagaaaacttgaacagccctggttctacaggaatcatcatttgcaacttgaaacgtcaatttctaatctgacagactattcaactatggaagagtggtgtgagcatatattaagtggcttaagctctttagattttcttcatctataagataatattataattctaaatttgtctgtcttcaatgtggataaaaatacagatatgatttggaatctatttgttggaatttaaaaaataaaacaaaaaacaaaaaacaagcagtgccctctaagtgagctaatgacatagaaaaccaggagaggagaggaaggctcgtggccgcagtgtggcatggcacccggccaagtagtaggattaatttggaaggtaggtgaccaaatgcagagctatcttttttcctctgagtaaacctaactttgttttcctgggactgttttgttttgctcagctaagaaaaactgacccagcccggttataaataaactcaggaacctcaaaagtcctctctcctctgcgagcaaacttgaatcaaacaggtcaaagccaacactggacctccaggccatcctagtaaggacttctaacgaaaactaaggctggtagatcattagcattgggacaggctcactttacagcgtttgaaaccattctgcacgccaacggtccctttgttaagcaatagtgatgatttttattccagatgttctgcatggacttgtgaaaaaaaactaaaataaacttggaaaaccctacagagagagcaagggggaaaggagatcactagtaatcttggatgaccttggacagatttttgccacataatcctcaagactacggggagagaagaaattgtaaaagagggttgttttaacctattcattatgtttatcagagtctttagggcatagatgagctttcagtcctgaaaggttagaaggcctgttctattacctacatggttaacagcaaaattgagatgagagaaagcagtattaaaagcaggtagatatgaagctagaagtggccagtcttactatgccagtaagacctcgtacaccttgcttctctgttcactaaaactttcatttctttcagatttcgactactgggattatgttgtccctgagcccaacctcaacgaggtggtatttgaagagacaacatgccagaatttggttaaaatgttggagaactgtctgtccagatcaaagcaaaccaaacttggttgctctaaggtcttggtccctgagaaactgacccagagaattgctcaagatgtcctgcggctctcgtccacggagccctgcggccttcggggttgtgttatgcacgtgaacttggagattgaaaatgtatgtaaaaagctggataggatcgtgtgtgatgctagcgtggtgccgacctttgagctcacgcttgtgtttaagcaggagagctgcccgtggaccagcctcaaggacttcttctttagcagaggtcgcttctcctcaggccttaagcgaactctgattctcagctcaggatttcgacttgtgaagaaaaaactgtactccctgattggaacgacagtcattgaagagtgctaaggaggaaaaacaattaaaggcccctaatgagtggataataaacctctgaagctatccagccagtcatttgtagtttgcctgcttgccctcaccaggaaatcccagatttaggcccttttccttctgtgtgtctcacccatagcaacccactttaaaggctgattttttttttttttgtccagcttttaagacataattcaagagagcaaattgcccacatatgtgtccatatgggatagaatggaataaggaggaaaaagaagcaatctcctttaattttatatttggtctcatatttagcaggtagtcttagcaacagagcaagatccactttgcctaatttgactttcatgtgcccagggcttgctgttattgtacccgcctctctctctctctctctctctctctctgtgtgtgtgtgtgtgtgtgtgtgtatatatatatatctgtctgtctctctctgtctctctgtctctctctctctcctcccctccctccctctctgagagccatcttttcaatgacaggacctgacaagatgatgtgcagccccatcaaaacatatagagatctgcactaccagcttcctccctgaatttgaagagcttttacacaagagggtacagttaaattgatccgctggattaaaacatcaccttacatagagcgaataaacatcacagtgacaagagcatggcttccctgcctggtggtctccccacacccgtgcccaggagacacgagaagtgtgcctcttactgcaatgattacatgatggctttcagt

SEQ ID NO: 92 - DNA, 4599 bp, Homo sapiens
  source 1..4599: mol_type = genomic DNA
gctactgcgaggagccggcgcagggtggcccgggaggggtgagcagggtgccgctggctgctggggtctgcaggtcaccgagtccccaggagaggggactcctaagaagccacctgcctgtgtttacccggcagcgagcgcgcaggcccccgcgaactcctggcaggtgagcgaagctgcggccttggtgggggcagcggggccggggggcgctccgcggggcaggtcgcttccaagttttgcgaactgttgagttagtttcaggttgcggctccgctcccagggctttgtcaaattgagttgcagaggccggttccacccctttggcaaaggagttcctccgctagttcgctcccgggacgccgggcattttaggagcctctgcacgactcgcctgcttttgcaaatccgtctcttctcgcctcattttttccagcgctcaggaaaggccgttgcgcctcgcgaaggaaacagagccgttgaccatggttgcaactggcagtttgagcagcaagaacccggccagcatttcagaattgctggactgtggctatcacccagagagcctgctaagtggtgagtgtccccgctgtccacccttctcccctcccaaggtcggtgcctctggatctattgggtgggcgggctttcgacttcctcccctacctctatgtaggaggtgccacaaaatccatctgaggagactcctttccaaattttctaacatcttctctccatgatagaaacaaaagtagcaaaagtagaatacccgagggctgcatctttctagtctggtgccagtaggccccttgggattaagttccttatcttacatgattgtgaagaacacagttatgtgtgttttagcaaatgtatttaccctgtaatcaatatcgcccctccccctgaattccgacgcacctatttgccaggtattcctcactctcccctgactccagggaactactgatctgatttttatcattccaagttagttttgcctttaggtaagtggaaccatacagtaggtattcttttgtgtctggtttctttcccgcagcctaatgtttttgagattcttttatgtccgttgcttaaatcagtgtgttcgtgtttattgtttattactgagtaggattttcagtcgtggatatatccattattccatttttaatgtgagaaggaaactgttgaacagcagcggtgcttgacttgctgtcatttggaattttaacagcagtttctaggtgaacacactgctctgacactaaagagctgtaagagcatagataaactgcttaagctcttcgtgtttttctcctttcaaggtgaagtggttgatgacctaatttacaggccagaactccaaacttacacgaagtggagaggaaaagccagtggctgatgaaggtggagtaggcttagagagaggagctgggagggcatctgcctgcaccttgttcatttgactccgtgtggtgtggcatcctgccaaggggtaggattaatttgaggtgtaggtgaccaaatgcagagctatctcttttcctttgagcccagctaattttatttccctaggactattttgttttgcttggctaagaaaaaccacccagccggttacaaatatcctcatgaacctcaaaggtcttctctgcacaatttttaattaaagaggctaaagctaaaattgggacctacaggccactccagtaaagacttaaagacacaaatttgatagatctttaatataactcttaaagctttaatacaactcttcaaacataatagtaaaaattctttattttacaatacatgtattcctacagttagaaagtaacaaccccttagtccaaaaaacaaagttataactttttattccagaagtaatgcatgttttctgtaaaacaaaattagaaaacccccataagcaagtaggagaaaagaaaagaaaatcaccaataatctgggataatcacaagtagatttttgatgcatagtcttcacggttgtgtgtgtgttttgtgtacacacataaatttaaagaaaagtggttacatcctatctactctatttatctaaacctttatttcatacatgaactttcattcatgagagcttaaattattcgctatattacctagctagttaacagttaattgaggtcagctgactctgaaacaaatacaagaaaaaggcagacatggtagatagaggtaaccaatgttgatatcttggtgttttttttctactcttaaattttttcttctgtatctaaaaagaaaactccatttcttacagattttgactactgggattatgttgttcctgaacccaacctcaacgaggtaatatttgaggaatcaacttgccagaatttggttaaaatgctggagaactgtctgtccaaatcaaagcaaactaaacttggttgctcaaaggtccttgtccctgagaaactgacccagagaattgctcaagatgtcctgcggctttcctcaacggagccctgcggcttgcgaggttgtgttatgcacgtgaacttggaaattgaaaatgtatgtaaaaagctggataggattgtgtgtgattctagcgtcgtacctacttttgagcttacacttgtgtttaagcaggagaactgctcatggactagcttcagggactttttctttagtagaggtcgcttctcctctggtttcaggagaactctgatcctcagctcaggatttcgacttgttaagaaaaaactttactcactgattggaacaacagtgattgaagggtcctaaaaagggaaaatatataaagattatttcatgattgggtagtaaaactattcagctagtcagctaaagtcatttgtagtttgccccacctgccctaaataagaaaccccaaatgtagtctcttttctttctgtgtttcacattcatagcaactgcagctaacaggctgattttctggcctttggagaagtgattcaaaatagtgtagattttctgcatagatcccatttttgtacagaattgaatgggatggaataggtaagcaaaagtagaagcccatttgagttttacatttgattccacaatttggtttcaggtaggcttggtaatagactatataaaccagatttgcctattttgattttcatatggcttttttttctgtaagttttcagaggattttttaaatcacagaatcatactaaatgatatttagcctatcaaaacttccaaaagcccacaccaccagttcctgactcaaatttgaagggtttttagacaggagggtaggattaagtaggtgagtttaattaaagcttaaccctaggtaagagtaaatgagaaatattacggcaataatggaactgcttcactgtttcttggtgacttcctcactctaatgttttaaagaggcaacaaaagcttgtggtgccatttcagtaaccacggtgttgttttagatgcctttataagctcagtttcccctgttcttaagtgttgaatactgtctttaaactagaaaaatgcaaaatattgaactgatatttttgtgtgtagttgattactcttccattgagtgaatgatgaatacctgtgaggataggaaattagttctgagatctagtccctctctgattcacttagtaatctatcctcttttcagtattacatgtgcttaatctcagatgaaccatttcaccatggcagtgttatctcatctctgggcttttctgggaattgaagtatctctccttaaccccaattgtcaagggtagtagctgtatactaccactttgaattattgaaacgggtcaatttacgaagtctgcattggctatggagatatggtttatagtacagcctagagaatgaaactcaccgtccagataaccatgcatgcacccagattttttccaccttggatacctgtcactagggaataataaaggcctgattttttgtcttattccaactaagtagatcattatctctttccttttttatgttaatgagagaatttagcctccactcaacaatgttcaattcagcaaggctttcatatccttgctgtgggtcgtggataaggagcttattcaggtttcctgccctagctattagctccacttcacatgctggagaccggcgtagggacagatgtattcatcctggtgttactgaaaaacaggtgtgatcctgttactgatactataagtgacctaaaatgtcactgttcaaattagccagtgttctaacaaactaaactcttcaaatgcttggaaagatactacaaagccaatctttatagaattgggccaagataaatctatgttgttttgcatggctattgttaagctccaaaggttcactgtgtttctgccgctgtcctggagttgtcaccactgactgggcaaggcttcttgggcatggatgtagaactgttgtccttttcccactaacagttatctttgactctcttgcctgttatgcttacaaaatggtgatggcttatggaaggctgttaaattaatattcctgttaaaggaaattaaagtttgtctatttttgacaataaaacattatatatttttaa

SEQ ID NO: 93 - DNA, 4163 bp, Homo sapiens
  source 1..4163: mol_type = genomic DNA
cgctcaggaaaggccgttgcgcctcgcgaaggaaacagagccgttgaccatggttgcaactggcagtttgagcagcaagaacccggccagcatttcagaattgctggactgtggctatcacccagagagcctgctaagtggtgagtgtccccgctgtccacccttctcccctcccaaggtcggtgcctctggatctattgggtgggcgggctttcgacttcctcccctacctctatgtaggaggtgccacaaaatccatctgaggagactcctttccaaattttctaacatcttctctccatgatagaaacaaaagtagcaaaagtagaatacccgagggctgcatctttctagtctggtgccagtaggccccttgggattaagttccttatcttacatgattgtgaagaacacagttatgtgtgttttagcaaatgtatttaccctgtaatcaatatcgcccctccccctgaattccgacgcacctatttgccaggtattcctcactctcccctgactccagggaactactgatctgatttttatcattccaagttagttttgcctttaggtaagtggaaccatacagtaggtattcttttgtgtctggtttctttcccgcagcctaatgtttttgagattcttttatgtccgttgcttaaatcagtgtgttcgtgtttattgtttattactgagtaggattttcagtcgtggatatatccattattccatttttaatgtgagaaggaaactgttgaacagcagcggtgcttgacttgctgtcatttggaattttaacagcagtttctaggtgaacacactgctctgacactaaagagctgtaagagcatagataaactgcttaagctcttcgtgtttttctcctttcaaggtgaagtggttgatgacctaatttacaggccagaactccaaacttacacgaagtggagaggaaaagccagtggctgatgaaggtggagtaggcttagagagaggagctgggagggcatctgcctgcaccttgttcatttgactccgtgtggtgtggcatcctgccaaggggtaggattaatttgaggtgtaggtgaccaaatgcagagctatctcttttcctttgagcccagctaattttatttccctaggactattttgttttgcttggctaagaaaaaccacccagccggttacaaatatcctcatgaacctcaaaggtcttctctgcacaatttttaattaaagaggctaaagctaaaattgggacctacaggccactccagtaaagacttaaagacacaaatttgatagatctttaatataactcttaaagctttaatacaactcttcaaacataatagtaaaaattctttattttacaatacatgtattcctacagttagaaagtaacaaccccttagtccaaaaaacaaagttataactttttattccagaagtaatgcatgttttctgtaaaacaaaattagaaaacccccataagcaagtaggagaaaagaaaagaaaatcaccaataatctgggataatcacaagtagatttttgatgcatagtcttcacggttgtgtgtgtgttttgtgtacacacataaatttaaagaaaagtggttacatcctatctactctatttatctaaacctttatttcatacatgaactttcattcatgagagcttaaattattcgctatattacctagctagttaacagttaattgaggtcagctgactctgaaacaaatacaagaaaaaggcagacatggtagatagaggtaaccaatgttgatatcttggtgttttttttctactcttaaattttttcttctgtatctaaaaagaaaactccatttcttacagattttgactactgggattatgttgttcctgaacccaacctcaacgaggtaatatttgaggaatcaacttgccagaatttggttaaaatgctggagaactgtctgtccaaatcaaagcaaactaaacttggttgctcaaaggtccttgtccctgagaaactgacccagagaattgctcaagatgtcctgcggctttcctcaacggagccctgcggcttgcgaggttgtgttatgcacgtgaacttggaaattgaaaatgtatgtaaaaagctggataggattgtgtgtgattctagcgtcgtacctacttttgagcttacacttgtgtttaagcaggagaactgctcatggactagcttcagggactttttctttagtagaggtcgcttctcctctggtttcaggagaactctgatcctcagctcaggatttcgacttgttaagaaaaaactttactcactgattggaacaacagtgattgaagggtcctaaaaagggaaaatatataaagattatttcatgattgggtagtaaaactattcagctagtcagctaaagtcatttgtagtttgccccacctgccctaaataagaaaccccaaatgtagtctcttttctttctgtgtttcacattcatagcaactgcagctaacaggctgattttctggcctttggagaagtgattcaaaatagtgtagattttctgcatagatcccatttttgtacagaattgaatgggatggaataggtaagcaaaagtagaagcccatttgagttttacatttgattccacaatttggtttcaggtaggcttggtaatagactatataaaccagatttgcctattttgattttcatatggcttttttttctgtaagttttcagaggattttttaaatcacagaatcatactaaatgatatttagcctatcaaaacttccaaaagcccacaccaccagttcctgactcaaatttgaagggtttttagacaggagggtaggattaagtaggtgagtttaattaaagcttaaccctaggtaagagtaaatgagaaatattacggcaataatggaactgcttcactgtttcttggtgacttcctcactctaatgttttaaagaggcaacaaaagcttgtggtgccatttcagtaaccacggtgttgttttagatgcctttataagctcagtttcccctgttcttaagtgttgaatactgtctttaaactagaaaaatgcaaaatattgaactgatatttttgtgtgtagttgattactcttccattgagtgaatgatgaatacctgtgaggataggaaattagttctgagatctagtccctctctgattcacttagtaatctatcctcttttcagtattacatgtgcttaatctcagatgaaccatttcaccatggcagtgttatctcatctctgggcttttctgggaattgaagtatctctccttaaccccaattgtcaagggtagtagctgtatactaccactttgaattattgaaacgggtcaatttacgaagtctgcattggctatggagatatggtttatagtacagcctagagaatgaaactcaccgtccagataaccatgcatgcacccagattttttccaccttggatacctgtcactagggaataataaaggcctgattttttgtcttattccaactaagtagatcattatctctttccttttttatgttaatgagagaatttagcctccactcaacaatgttcaattcagcaaggctttcatatccttgctgtgggtcgtggataaggagcttattcaggtttcctgccctagctattagctccacttcacatgctggagaccggcgtagggacagatgtattcatcctggtgttactgaaaaacaggtgtgatcctgttactgatactataagtgacctaaaatgtcactgttcaaattagccagtgttctaacaaactaaactcttcaaatgcttggaaagatactacaaagccaatctttatagaattgggccaagataaatctatgttgttttgcatggctattgttaagctccaaaggttcactgtgtttctgccgctgtcctggagttgtcaccactgactgggcaaggcttcttgggcatggatgtagaactgttgtccttttcccactaacagttatctttgactctcttgcctgttatgcttacaaaatggtgatggcttatggaaggctgttaaattaatattcctgttaaaggaaattaaagtttgtctatttttgacaataaaacattatatatttttaa

SEQ ID NO: 94 - DNA, 582 bp, Homo sapiens
  source 1..582: mol_type = genomic DNA
atggttgcaactggcagtttgagcagcaagaacccggccagcatttcagaattgctggactgtggctatcacccagagagcctgctaagtgattttgactactgggattatgttgttcctgaacccaacctcaacgaggtaatatttgaggaatcaacttgccagaatttggttaaaatgctggagaactgtctgtccaaatcaaagcaaactaaacttggttgctcaaaggtccttgtccctgagaaactgacccagagaattgctcaagatgtcctgcggctttcctcaacggagccctgcggcttgcgaggttgtgttatgcacgtgaacttggaaattgaaaatgtatgtaaaaagctggataggattgtgtgtgattctagcgtcgtacctacttttgagcttacacttgtgtttaagcaggagaactgctcatggactagcttcagggactttttctttagtagaggtcgcttctcctctggtttcaggagaactctgatcctcagctcaggatttcgacttgttaagaaaaaactttactcactgattggaacaacagtgattgaagggtcctaa

SEQ ID NO: 95 - protein, 193 aa, Homo sapiens
MVATGSLSSKNPASISELLDCGYHPESLLSDFDYWDYVVPEPNLNEVIFEESTCQNLVKMLENCLSKSKQTKLGCSKVLVPEKLTQRIAQDVLRLSSTEPCGLRGCVMHVNLEIENVCKKLDRIVCDSSVVPTFELTLVFKQENCSWTSFRDFFFSRGRFSSGFRRTLILSSGFRLVKKKLYSLIGTTVIEGS

SEQ ID NO: 96 - DNA, 20 bp, Synthetic construct
  source 1..20: mol_type = other DNA
tgctggactgtggctatcac

SEQ ID NO: 97 - DNA, 21 bp, synthetic construct
  source 1..21: mol_type = other DNA
acaaggacctttgagcaacca

SEQ ID NO: 98 - DNA, 20 bp, Synthetic construct
  source 1..20: mol_type = other DNA
gaggagcctgtgcacttctt

SEQ ID NO: 99 - DNA, 20 bp, Synthetic construct
  source 1..20: mol_type = other DNA
cacacccagcctgtacactt

SEQ ID NO: 100 - DNA, 18 bp, synthetic construct
  source 1..18: mol_type = other DNA
  misc_feature 1..18: note = Forward primer to detect the expression of DIR mRNA
tgcctcggtttacccttc

SEQ ID NO: 101 - DNA, 24 bp, synthetic construct
  source 1..24: mol_type = other DNA
  misc_feature 1..24: note = Reverse primer to detect the expression of DIR mRNA
aagatgttagaaaatttggaaagg

SEQ ID NO: 102 - DNA, 20 bp, synthetic construct
  source 1..20: mol_type = other DNA
  misc_feature 1..20: note = Forward primer to detect the expression of mDIR mRNA
acgggcagtttgagcagtaa

SEQ ID NO: 103 - DNA, 21 bp, synthetic construct
  source 1..21: mol_type = other DNA
  misc_feature 1..21: note = Reverse primer to detect the expression of mDIR mRNA
cacaagtaagcgcatgtcttt

SEQ ID NO: 104 - DNA, 18 bp, synthetic construct
  source 1..18: mol_type = other DNA
  misc_feature 1..18: note = Forward primer to detect the expression of mDDIT4L mRNA
tgcctcggtttacccttc

SEQ ID NO: 105 - DNA, 23 bp, Synthetic construct
  source 1..23: mol_type = other DNA
  misc_feature 1..23: note = Reverse primer to detect the expression of mDDIT4L mRNA
ccagctttttacatacattttca

SEQ ID NO: 106 - DNA, 30 bp, synthetic construct
  source 1..30: mol_type = other DNA
  misc_feature 1..30: note = Forward primer to detect the expression of GAPDH mRNA
tatgtcgtggagtctactggtgtcttcacc

SEQ ID NO: 107 - DNA, 28 bp, synthetic construct
  source 1..28: mol_type = other DNA
  misc_feature 1..28: note = Reverse primer to detect the expression of GAPDH mRNA
gttgtcatatttctcgtggttcacaccc

(19) United States
(12) Patent Application Publication
(10) Pub. No.: US 2025/0060376 A1
(43) Pub. Date: Feb. 20, 2025

(54) DIGESTED DDIT4L PRODUCT AS DIAGNOSTIC MARKER FOR ALZHEIMER'S DISEASE

(71) Applicant: PLASMARKER BIOTECHNOLOGY CO., LTD, Suzhou (CN)

(72) Inventors: Kai-cheng LI, Suzhou (CN); Pu YOU, Suzhou (CN)

(73) Assignee: PLASMARKER BIOTECHNOLOGY CO., LTD, Suzhou (CN)

(21) Appl. No.: 18/930,376

(22) Filed: Oct. 29, 2024

Related U.S. Application Data
(63) Continuation-in-part of application No. PCT/CN2023/091199, filed on Apr. 27, 2023.

(30) Foreign Application Priority Data
Apr. 29, 2022 (WO) PCT/CN2022/090220

(51) Int. Cl. G01N 33/68; C12Q 1/6883; G01N 33/53

(52) U.S. Cl. CPC G01N 33/6896; C12Q 1/6883; G01N 33/53; G01N 2333/4709; G01N 2800/2821; G01N 2800/2835; G01N 2800/285; G01N 2800/52

(57) ABSTRACT

The present application relates to a digested DDIT4L product as a diagnostic marker for Alzheimer's disease, and use thereof in diagnosing Alzheimer's disease. In particular, the present application relates to use of a substance for detecting a digested intron retention (DIR) product encoding DNA-damage-inducible transcript 4 like (DDIT4L) in a sample of a subject in preparing a product for diagnosing Alzheimer's disease or a mild cognitive disorder and/or assessing (e.g., grading or staging) cognitive disorder progression, a related product thereof, and a method for screening a medicament using the DIR product.

### CROSS-REFERENCE TO RELATED APPLICATIONS

This application is a Continuation-in-part of and claims priority benefit to Patent Cooperation Treaty application PCT/CN2023/091199, filed Apr. 27, 2023, which claims the benefit of Patent Cooperation Treaty application PCT/CN2022/090220, filed Apr. 29, 2022. Priority is claimed to these applications and the disclosures of these priority applications are considered part of the disclosure of this application and are incorporated herein by reference in their entireties.

### INCORPORATION OF SEQUENCE LISTING

This application contains a sequence listing which has been submitted electronically in XML format and is hereby incorporated by reference in its entirety. Said XML copy, created Oct. 25, 2024, is named “0245-PA-009US_Sequence listing.xml” and is 151,587 bytes in size.

### TECHNICAL FIELD

The present application relates to the field of biomedicine, and in particular to application of a DNA damage-inducible transcript 4-like (DDIT4L) intron retention spliced product (DIR) as a blood marker for presenile dementia or mild cognitive impairment, as well as a product and method corresponding thereto.

### BACKGROUND

Presenile dementia, also known as Alzheimer's disease (AD), is a neurodegenerative disease with the highest incidence among diseases that harm the human brain. It causes great suffering to patients themselves, and also brings a heavy burden to their families and the society.

According to “World Alzheimer Report 2017” from the Alzheimer's Disease International (ADI), the cost of taking caring of patients with dementia across the world has increased from US$604 billion in 2010 to US$1,000 billion in 2017, with a growth rate of 65.6%. There are 5 million AD patients in the United States, and caregivers need to spend 18 billion hours a year to take care of them. In 2017, there were 50 million AD patients around the world, and this number will continue to increase with the increase of the world's aging population. In the context where no medicament is available for treating, delaying or stopping the progression of the disease at present, the number of patients is expected to double every 20 years, reaching 82 million in 2030 and 150 million in 2050.

According to “China Development Report 2020: Development Trends in and Policies of Population Aging in China” released by the China Development Foundation (a national social organization initiated by the Development Research Center of the State Council), elderly people aged 65 and above in China are to be about 180 million by 2020, accounting for about 13% of the total population; above 210 million by 2025 when the “14th Five-Year Plan” is completed, accounting for about 15% of the total population; and up to 310 million and nearly 380 million in 2035 and 2050, accounting for up to 22.3% and 27.9% of the total population, respectively.

The incidence rate of AD is age-related and ranges from 2% to 4% of the incidence of the whole population. In particular, for middle-aged and elderly people aged over 65 years old, the prevalence rate is 5% and increases exponentially with the increase of age. Based on analysis, the prevalence rate can increase to 25% for elderly people aged over 85 years old, and is as high as 60% for those over 95 years old. China has gradually entered an aging society. According to statistics, there are nearly 10 million AD patients in China, and more than 300,000 new cases emerge every year.

The main symptom of AD is cognitive functional impairment. As it turns out, the cognitive function impairment is caused by synaptic and neuronal damage, and abnormal neural circuit structure and function. At present, the main reasons for the continuous failure of clinical research on new drugs for AD lie in unclear mechanism of the synaptic and neuronal damage, and lack of cell/animal models approaching the pathophysiological characteristics of human diseases (such as neurodegeneration, etc.), leading to slow progress of researches on effective methods for preventing and treating AD, and on early diagnostic markers capable of reflecting disease prognosis.

Metabolic disorders may be one of the main pathogenesis of AD. In AD patients, abnormal lipid metabolism revealed by cholesterol or the like and abnormal glucose metabolism revealed by glucose or the like are the most common metabolic disorders, which also include energy metabolism disorders like cerebral ischemia and hypoxia caused by cerebral circulation insufficiency, as represented by degenerative changes in cerebral small blood vessels. However, how the abnormal lipid metabolism and the abnormal glucose metabolism in brain affect the occurrence and development course of AD are unclear in mechanism. Mitochondria are key organelles for the metabolism of main energy substances, and their dysfunction can lead to abnormal energy metabolism and also damage the functions of neurons and the endothelial cells of cerebral small blood vessels. However, in the incidence and development of early AD, the relationship between mitochondrial dysfunction and abnormal lipid metabolism and glucose metabolism is not clear.

Mild cognitive impairment (MCI), a syndrome involving objective cognitive decline, is a state between mild dementia and healthy aging, with memory impairment as the prominent manifestation. It may involve other cognitive declines to a certain degree and normal competence for daily activities, and thus does not meet the diagnostic criteria for dementia. 10% to 15% of MCI are converted into AD every year, which is 10 times that of normal controls. As a transitional state from normal aging to dementia, MCI has attracted more and more attention, and studying MCI is an important part in the early diagnosis and early intervention of AD.

Current diagnostic markers for AD mainly include detection of Aβ and Tau contents in cerebrospinal fluid, PET and MRI neuroimaging means, etc. Although these methods have higher or even very high diagnostic efficacy, they also have shortcomings such as difficulty in operation, high cost, and weak correlation between injury and disease outcome. Accordingly, there is an urgent need to develop a new marker that is simple, easy to implement, cost-effective, and suitable for large-scale population screening, and that can be better used for early diagnosis of AD.

### SUMMARY OF THE INVENTION

The present application provides a novel blood marker for Alzheimer's disease (AD) or mild cognitive impairment (MCI).

In one aspect, the present application provides use of a DNA damage-inducible transcript 4-like (DDIT4L) intron retention spliced product (DIR), and/or a nucleic acid encoding the DIR, in diagnosis of a disease and/or in evaluation of progression of the disease, wherein the disease comprises cognitive impairment.

In one aspect, the present application provides use of a DNA damage-inducible transcript 4-like (DDIT4L) intron retention spliced product (DIR), and/or a nucleic acid encoding the DIR, in diagnosis of a disease and/or in evaluation of progression of the disease, wherein the disease comprises a neurodegenerative disease.

In some embodiments, the DIR comprises the amino acid sequence as set forth in SEQ ID NO: 1.

In some embodiments, the nucleic acid encoding the DIR comprises a nucleotide sequence as set forth in SEQ ID NO: 8.

In some embodiments, the nucleic acid encoding the DIR comprises a nucleotide sequence as set forth in SEQ ID NO: 93.

In some embodiments, the cognitive impairment comprises cognitive impairment caused by normal aging, Lewy body dementia (LBD), frontotemporal dementia and/or vascular dementia.

In some embodiments, the cognitive impairment induces diseases comprising Alzheimer's disease, multi-infarct type, Parkinson's disease, AIDS and/or Creutzfeldt-Jakob disease (CJD).

In some embodiments, the cognitive impairment comprises mild cognitive impairment (MCI), intermediate cognitive impairment and late cognitive impairment.

In some embodiments, the cognitive impairment comprises multi-domain amnestic MCI (aMCI-m).

In some embodiments, the neurodegenerative disease comprises an acute neurodegenerative disease and a chronic neurodegenerative disease.

In some embodiments, the neurodegenerative disease comprises a neurodegenerative disease caused by neuronal death and glial cell homeostasis, a neurodegenerative disease caused by aging, a neurodegenerative disease caused by affected CNS cell function, a neurodegenerative disease caused by abnormal intercellular communication and/or a neurodegenerative disease caused by impaired cell mobility.

In some embodiments, the neurodegenerative disease comprises Alzheimer's disease, Parkinson's disease, multiple sclerosis (MS), amyotrophic lateral sclerosis (ALS) and/or Huntington's disease (HD).

In some embodiments, the neurodegenerative disease comprises presenile dementia.

In some embodiments, the neurodegenerative disease comprises early presenile dementia, intermediate presenile dementia and/or late presenile dementia.

In another aspect, the present application provides use of a substance for detecting a DNA damage-inducible transcript 4-like DDIT4L intron retention spliced product DIR, and/or a nucleic acid encoding said DIR, in preparation of a product for diagnosis of a disease and/or in evaluation of progression of said disease, wherein said disease comprises cognitive impairment.

In another aspect, the present application provides use of a substance for detecting a DNA damage-inducible transcript 4-like (DDIT4L) intron retention spliced product (DIR), and/or a nucleic acid encoding the DIR, in preparation of a product for diagnosis of a disease and/or in evaluation of progression of the disease, wherein the disease comprises a neurodegenerative disease.

In another aspect, the present application provides a diagnostic kit, comprising the substance capable of detecting a DNA damage-inducible transcript 4-like (DDIT4L) intron retention spliced product (DIR), and/or a nucleic acid encoding the DIR, as described in the present application, wherein the diagnostic kit is for use in diagnosis of cognitive impairment, and/or evaluation of progression of the cognitive impairment; and/or for use in diagnosis of a neurodegenerative disease, and/or evaluation of progression of the neurodegenerative disease.

In some embodiments, the diagnostic kit comprises an instruction manual recording a method of using the diagnostic kit for diagnosis of cognitive impairment, and/or evaluation of progression of the cognitive impairment; and/or for diagnosis of a neurodegenerative disease, and/or evaluation of progression of the neurodegenerative disease.

In some embodiments, the DIR comprises the amino acid sequence as set forth in SEQ ID NO: 1.

In some embodiments, the nucleic acid encoding the DIR comprises a nucleotide sequence as set forth in SEQ ID NO: 8.

In some embodiments, the nucleic acid encoding the DIR comprises a nucleotide sequence as set forth in SEQ ID NO: 93.

In some embodiments, the cognitive impairment comprises cognitive impairment caused by normal aging, Lewy body dementia (LBD), frontotemporal dementia and/or vascular dementia.

In some embodiments, the cognitive impairment induces diseases comprising Alzheimer's disease, multi-infarct type, Parkinson's disease, AIDS and/or Creutzfeldt-Jakob disease (CJD).

In some embodiments, the cognitive impairment comprises mild cognitive impairment (MCI), intermediate cognitive impairment and late cognitive impairment.

In some embodiments, the cognitive impairment comprises multi-domain amnestic MCI (aMCI-m).

In some embodiments, the neurodegenerative disease comprises an acute neurodegenerative disease and a chronic neurodegenerative disease.

In some embodiments, the neurodegenerative disease comprises a neurodegenerative disease caused by neuronal death and glial cell homeostasis, a neurodegenerative disease caused by aging, a neurodegenerative disease caused by affected CNS cell function, a neurodegenerative disease caused by abnormal intercellular communication and/or a neurodegenerative disease caused by impaired cell mobility.

In some embodiments, the neurodegenerative disease comprises Alzheimer's disease, Parkinson's disease, multiple sclerosis (MS), amyotrophic lateral sclerosis (ALS) and/or Huntington's disease (HD).

In some embodiments, the neurodegenerative disease comprises presenile dementia.

In some embodiments, the neurodegenerative disease comprises early presenile dementia, intermediate presenile dementia and/or late presenile dementia.

In some embodiments, the substance comprises a reagent and/or an apparatus capable of detecting the DIR, and/or the nucleic acid encoding the DIR.

In some embodiments, the substance comprises a reagent for specifically detecting the DIR, and/or the nucleic acid encoding the DIR.

In some embodiments, the substance comprises an antigen-binding protein capable of specifically binding to the DIR, a probe capable of specifically binding to the DIR, a primer capable of specifically amplifying the nucleic acid encoding the DIR, a gene chip capable of specifically detecting the nucleic acid encoding the DIR, an aptamer capable of specifically binding to the nucleic acid encoding the DIR, and/or a guide RNA capable of specifically binding to the nucleic acid encoding the DIR.

In some embodiments, the antigen-binding protein comprises LCDR2, which comprises the amino acid sequence as set forth in SEQ ID NO: 74.

In some embodiments, the LCDR2 comprises the amino acid sequence as set forth in SEQ ID NO: 26 or 16.

In some embodiments, the antigen-binding protein comprises LCDR1, which comprises the amino acid sequence as set forth in SEQ ID NO: 73.

In some embodiments, the LCDR1 comprises the amino acid sequence as set forth in any one of SEQ ID NOs: 25, 52 and 15.

In some embodiments, the antigen-binding protein comprises LCDR3, which comprises the amino acid sequence as set forth in SEQ ID NO: 75.

In some embodiments, the LCDR3 comprises the amino acid sequence as set forth in any one of SEQ ID NOs: 27, 53, 67 and 17.

In some embodiments, the antigen-binding protein comprises LCDR1, LCDR2, and LCDR3, wherein
a) the LCDR1 comprises the amino acid sequence as set forth in SEQ ID NO: 25, the LCDR2 comprises the amino acid sequence as set forth in SEQ ID NO: 26, and the LCDR3 comprises the amino acid sequence as set forth in SEQ ID NO: 27;
b) the LCDR1 comprises the amino acid sequence as set forth in SEQ ID NO: 52, the LCDR2 comprises the amino acid sequence as set forth in SEQ ID NO: 26, and the LCDR3 comprises the amino acid sequence as set forth in SEQ ID NO: 53;
c) the LCDR1 comprises the amino acid sequence as set forth in SEQ ID NO: 52, the LCDR2 comprises the amino acid sequence as set forth in SEQ ID NO: 26, and the LCDR3 comprises the amino acid sequence as set forth in SEQ ID NO: 67; or,
d) the LCDR1 comprises the amino acid sequence as set forth in SEQ ID NO: 15, the LCDR2 comprises the amino acid sequence as set forth in SEQ ID NO: 16, and the LCDR3 comprises the amino acid sequence as set forth in SEQ ID NO: 17.

In some embodiments, the antigen-binding protein comprises HCDR1, which comprises the amino acid sequence as set forth in SEQ ID NO: 70.

In some embodiments, the HCDR1 comprises the amino acid sequence as set forth in any one of SEQ ID NOs: 20, 35, 56 and 10.

In some embodiments, the antigen-binding protein comprises HCDR2, which comprises the amino acid sequence as set forth in SEQ ID NO: 71.

In some embodiments, the HCDR2 comprises the amino acid sequence as set forth in any one of SEQ ID NOs: 21, 36, 42, 48 and 11.

In some embodiments, the antigen-binding protein comprises HCDR3, which comprises the amino acid sequence as set forth in SEQ ID NO: 70.

In some embodiments, the HCDR3 comprises the amino acid sequence as set forth in any one of SEQ ID NOs: 22, 30, 37, 43, 49, 57, 62 and 12.

In some embodiments, the antigen-binding protein comprises HCDR1, HCDR2, and HCDR3, wherein
a) the HCDR1 comprises the amino acid sequence as set forth in SEQ ID NO: 20, the HCDR2 comprises the amino acid sequence as set forth in SEQ ID NO: 21, and the HCDR3 comprises the amino acid sequence as set forth in SEQ ID NO: 22;
b) the HCDR1 comprises the amino acid sequence as set forth in SEQ ID NO: 20, the HCDR2 comprises the amino acid sequence as set forth in SEQ ID NO: 21, and the HCDR3 comprises the amino acid sequence as set forth in SEQ ID NO: 30;
c) the HCDR1 comprises the amino acid sequence as set forth in SEQ ID NO: 35, the HCDR2 comprises the amino acid sequence as set forth in SEQ ID NO: 36, and the HCDR3 comprises the amino acid sequence as set forth in SEQ ID NO: 37;
d) the HCDR1 comprises the amino acid sequence as set forth in SEQ ID NO: 20, the HCDR2 comprises the amino acid sequence as set forth in SEQ ID NO: 42, and the HCDR3 comprises the amino acid sequence as set forth in SEQ ID NO: 43;
e) the HCDR1 comprises the amino acid sequence as set forth in SEQ ID NO: 20, the HCDR2 comprises the amino acid sequence as set forth in SEQ ID NO: 48, and the HCDR3 comprises the amino acid sequence as set forth in SEQ ID NO: 49;
f) the HCDR1 comprises the amino acid sequence as set forth in SEQ ID NO: 56, the HCDR2 comprises the amino acid sequence as set forth in SEQ ID NO: 36, and the HCDR3 comprises the amino acid sequence as set forth in SEQ ID NO: 57;
g) the HCDR1 comprises the amino acid sequence as set forth in SEQ ID NO: 35, the HCDR2 comprises the amino acid sequence as set forth in SEQ ID NO: 36, and the HCDR3 comprises the amino acid sequence as set forth in SEQ ID NO: 62;
h) the HCDR1 comprises the amino acid sequence as set forth in SEQ ID NO: 35, the HCDR2 comprises the amino acid sequence as set forth in SEQ ID NO: 42, and the HCDR3 comprises the amino acid sequence as set forth in SEQ ID NO: 37;
i) the HCDR1 comprises the amino acid sequence as set forth in SEQ ID NO: 56, the HCDR2 comprises the amino acid sequence as set forth in SEQ ID NO: 36, and the HCDR3 comprises the amino acid sequence as set forth in SEQ ID NO: 57; or
j) the HCDR1 comprises the amino acid sequence as set forth in SEQ ID NO: 10, the HCDR2 comprises the amino acid sequence as set forth in SEQ ID NO: 11, and the HCDR3 comprises the amino acid sequence as set forth in SEQ ID NO: 12.

In some embodiments, the antigen-binding protein comprises a heavy chain variable region VH, which comprises the amino acid sequence as set forth in any one of SEQ ID NOs: 13, 23, 31, 38, 44, 50, 58, 63 and 65.

In some embodiments, the antigen-binding protein comprises a light chain variable region VL, which comprises the amino acid sequence as set forth in any one of SEQ ID NOs: 18, 28, 33, 40, 46, 54, 60 and 68.

In some embodiments, the antigen-binding protein comprises a heavy chain variable region VH and a light chain variable region VL, wherein:
a) the VH comprises the amino acid sequence as set forth in SEQ ID NO: 23 and the VL comprises the amino acid sequence as set forth in SEQ ID NO: 28;
b) the VH comprises the amino acid sequence as set forth in SEQ ID NO: 31 and the VL comprises the amino acid sequence as set forth in SEQ ID NO: 33;
c) the VH comprises the amino acid sequence as set forth in SEQ ID NO: 38 and the VL comprises the amino acid sequence as set forth in SEQ ID NO: 40;
d) the VH comprises the amino acid sequence as set forth in SEQ ID NO: 44 and the VL comprises the amino acid sequence as set forth in SEQ ID NO: 46;
e) the VH comprises the amino acid sequence as set forth in SEQ ID NO: 50 and the VL comprises the amino acid sequence as set forth in SEQ ID NO: 54;
f) the VH comprises the amino acid sequence as set forth in SEQ ID NO: 58 and the VL comprises the amino acid sequence as set forth in SEQ ID NO: 60;
g) the VH comprises the amino acid sequence as set forth in SEQ ID NO: 63 and the VL comprises the amino acid sequence as set forth in SEQ ID NO: 60;
h) the VH comprises the amino acid sequence as set forth in SEQ ID NO: 65 and the VL comprises the amino acid sequence as set forth in SEQ ID NO: 54;
i) the VH comprises the amino acid sequence as set forth in SEQ ID NO: 58 and the VL comprises the amino acid sequence as set forth in SEQ ID NO: 68; or
j) the VH comprises the amino acid sequence as set forth in SEQ ID NO: 13, and the VL comprises the amino acid sequence as set forth in SEQ ID NO: 18.

In some embodiments, the antigen-binding protein comprises a heavy chain constant region, which comprises an IgG-derived constant region.

In some embodiments, the heavy chain constant region comprises a constant region derived from a protein selected from the group consisting of: IgG1, IgG2, IgG3 and IgG4.

In some embodiments, the antigen-binding protein comprises a light chain constant region, which comprises an Igκ-derived constant region or an Igλ-derived constant region.

In some embodiments, the light chain constant region comprises a human Igκ-derived constant region.

In some embodiments, the antigen-binding protein comprises an antibody or an antigen-binding fragment thereof.

In some embodiments, the antigen-binding fragment is selected from the group consisting of: Fab, Fab′, F(ab)2, an Fv fragment, F(ab′)2, scFv, di-scFv, VHH and/or dAb.

In some embodiments, the antibody is selected from the group consisting of: a monoclonal antibody, a murine antibody and a chimeric antibody.

In some embodiments, the product comprises a diagnostic kit.

In another aspect, the present application provides a method for diagnosing cognitive impairment, and/or evaluating progression of the cognitive impairment, comprising the step of: detecting, from a sample of a subject in need thereof, a content of a DNA damage-inducible transcript 4-like (DDIT4L) intron retention spliced product (DIR), and/or of a nucleic acid encoding the DIR.

In another aspect, the present application provides a method for diagnosing a neurodegenerative disease, and/or evaluating progression of the neurodegenerative disease, comprising the step of: detecting, from a sample of a subject in need thereof, a content of a DNA damage-inducible transcript 4-like (DDIT4L) intron retention spliced product (DIR), and/or of a nucleic acid encoding the DIR.

In some embodiments, the DIR comprises the amino acid sequence as set forth in SEQ ID NO: 1.

In some embodiments, the nucleic acid encoding the DIR comprises a nucleotide sequence as set forth in SEQ ID NO: 8.

In some embodiments, the nucleic acid encoding the DIR comprises a nucleotide sequence as set forth in SEQ ID NO: 93.

In some embodiments, the cognitive impairment comprises cognitive impairment caused by normal aging, Lewy body dementia (LBD), frontotemporal dementia and/or vascular dementia.

In some embodiments, the cognitive impairment-inducing diseases comprise Alzheimer's disease, multi-infarct type, Parkinson's disease, AIDS and/or Creutzfeldt-Jakob disease (CJD).

In some embodiments, the cognitive impairment comprises mild cognitive impairment (MCI), intermediate cognitive impairment and late cognitive impairment.

In some embodiments, the cognitive impairment comprises multi-domain amnestic MCI (aMCI-m).

In some embodiments, the neurodegenerative disease comprises an acute neurodegenerative disease and a chronic neurodegenerative disease.

In some embodiments, the neurodegenerative disease comprises a neurodegenerative disease caused by neuronal death and glial cell homeostasis, a neurodegenerative disease caused by aging, a neurodegenerative disease caused by affected CNS cell function, a neurodegenerative disease caused by abnormal intercellular communication and/or a neurodegenerative disease caused by impaired cell mobility.

In some embodiments, the neurodegenerative disease comprises Alzheimer's disease, Parkinson's disease, multiple sclerosis (MS), amyotrophic lateral sclerosis (ALS) and/or Huntington's disease (HD).

In some embodiments, the neurodegenerative disease comprises presenile dementia.

In some embodiments, the neurodegenerative disease comprises early presenile dementia, intermediate presenile dementia and/or late presenile dementia.

In some embodiments, the subject comprises a mammal.

In some embodiments, the subject comprises a tumor patient.

In some embodiments, the subject is in an old age stage.

In some embodiments, the sample comprises a blood sample and/or a tissue or cell sample.

In some embodiments, the sample comprises whole blood, serum, plasma, and/or cerebrospinal fluid.

In some embodiments, the method comprises: comparing the content of the DIR and/or of the nucleic acid encoding the DIR in the sample of the subject with a normal control value, wherein the normal control value comprises a content of the DIR and/or of the nucleic acid encoding the DIR in a normal subject.

In some embodiments, the normal subject does not suffer from the cognitive impairment and/or the neurodegenerative disease.

In some embodiments, when the content of the DIR and/or of the nucleic acid encoding the DIR in the sample of the subject is significantly higher than the normal control value, the subject is diagnosed with the cognitive impairment and/or the neurodegenerative disease.

In some embodiments, the method comprises: comparing the content of the DIR and/or of the nucleic acid encoding the DIR in the sample of the subject with an early control value, wherein the early control value comprises a previously measured content of the DIR and/or of the nucleic acid encoding the DIR in the same subject.

In some embodiments, the subject has been diagnosed with the cognitive impairment and/or the neurodegenerative disease.

In some embodiments, when the content of the DIR and/or of the nucleic acid encoding the DIR in the sample of the subject is significantly higher than the early control value, the subject is diagnosed with aggravated progression of the cognitive impairment and/or the neurodegenerative disease.

In some embodiments, the method further comprises the step of detecting a content of a marker associated with the cognitive impairment and/or the neurodegenerative disease in a sample derived from the subject.

In some embodiments, the marker associated with the cognitive impairment and/or the neurodegenerative disease comprises AD7C-NTP, pTau-181, pTau-217, Aβ40 and/or Aβ42.

In some embodiments, the method further comprises the step of observing brain imaging of the subject.

In another aspect, the present application provides a system for diagnosing cognitive impairment, and/or evaluating progression of the cognitive impairment, comprising a substance for detecting a DNA damage-inducible transcript 4-like (DDIT4L) intron retention spliced product (DIR), and/or a nucleic acid encoding the DIR.

In another aspect, the present application provides a system for diagnosing a neurodegenerative disease, and/or evaluating progression of the neurodegenerative disease. The system comprises a substance for detecting a DNA damage-inducible transcript 4-like (DDIT4L) intron retention spliced product (DIR), and/or a nucleic acid encoding the DIR.

In some embodiments, the DIR comprises the amino acid sequence as set forth in SEQ ID NO: 1.

In some embodiments, the nucleic acid encoding the DIR comprises a nucleotide sequence as set forth in SEQ ID NO: 8.

In some embodiments, the nucleic acid encoding the DIR comprises a nucleotide sequence as set forth in SEQ ID NO: 93.

In some embodiments, the cognitive impairment comprises cognitive impairment caused by normal aging, Lewy body dementia (LBD), frontotemporal dementia and/or vascular dementia.

In some embodiments, the cognitive impairment-inducing diseases comprise Alzheimer's disease, multi-infarct type, Parkinson's disease, AIDS and/or Creutzfeldt-Jakob disease (CJD).

In some embodiments, the cognitive impairment comprises mild cognitive impairment (MCI), intermediate cognitive impairment and late cognitive impairment.

In some embodiments, the cognitive impairment comprises multi-domain amnestic MCI (aMCI-m).

In some embodiments, the neurodegenerative disease comprises an acute neurodegenerative disease and a chronic neurodegenerative disease.

In some embodiments, the neurodegenerative disease comprises a neurodegenerative disease caused by neuronal death and glial cell homeostasis, a neurodegenerative disease caused by aging, a neurodegenerative disease caused by affected CNS cell function, a neurodegenerative disease caused by abnormal intercellular communication and/or a neurodegenerative disease caused by impaired cell mobility.

In some embodiments, the neurodegenerative disease comprises Alzheimer's disease, Parkinson's disease, multiple sclerosis (MS), amyotrophic lateral sclerosis (ALS) and/or Huntington's disease (HD).

In some embodiments, the neurodegenerative disease comprises presenile dementia.

In some embodiments, the neurodegenerative disease comprises early presenile dementia, intermediate presenile dementia and/or late presenile dementia.

In some embodiments, the system exists in a kit form.

In some embodiments, the system comprises a collection module capable of collecting a sample from a subject in need thereof.

In some embodiments, the subject comprises a mammal.

In some embodiments, the subject comprises a tumor patient.

In some embodiments, the subject is in an old age stage.

In some embodiments, the sample comprises a blood sample and/or a tissue or cell sample.

In some embodiments, the sample comprises whole blood, serum, plasma, and/or cerebrospinal fluid.

In some embodiments, the system comprises a detection module capable of detecting a content value of the DIR and/or of the nucleic acid encoding the DIR.

In some embodiments, the detection module detects a content value of the DIR and/or of the nucleic acid encoding the DIR in the sample acquired by the collection module.

In some embodiments, the detection module comprises a substance capable of detecting a content value of the DIR and/or of the nucleic acid encoding the DIR.

In some embodiments, the substance comprises an antigen-binding protein capable of specifically binding to the DIR, a probe capable of specifically binding to the DIR, a primer capable of specifically amplifying the nucleic acid encoding the DIR, a gene chip capable of specifically detecting the nucleic acid encoding the DIR, an aptamer capable of specifically binding to the nucleic acid encoding the DIR, and/or a guide RNA capable of specifically binding to the nucleic acid encoding the DIR.

In some embodiments, the antigen-binding protein comprises LCDR2, which comprises the amino acid sequence as set forth in SEQ ID NO: 74.

In some embodiments, the LCDR2 comprises the amino acid sequence as set forth in SEQ ID NO: 26 or 16.

In some embodiments, the antigen-binding protein comprises LCDR1, which comprises the amino acid sequence as set forth in SEQ ID NO: 73.

In some embodiments, the LCDR1 comprises the amino acid sequence as set forth in any one of SEQ ID NOs: 25, 52 and 15.

In some embodiments, the antigen-binding protein comprises LCDR3, which comprises the amino acid sequence as set forth in SEQ ID NO: 75.

In some embodiments, the LCDR3 comprises the amino acid sequence as set forth in any one of SEQ ID NOs: 27, 53, 67 and 17.

In some embodiments, the antigen-binding protein comprises LCDR1, LCDR2, and LCDR3, wherein
a) the LCDR1 comprises the amino acid sequence as set forth in SEQ ID NO: 25, the LCDR2 comprises the amino acid sequence as set forth in SEQ ID NO: 26, and the LCDR3 comprises the amino acid sequence as set forth in SEQ ID NO: 27;
b) the LCDR1 comprises the amino acid sequence as set forth in SEQ ID NO: 52, the LCDR2 comprises the amino acid sequence as set forth in SEQ ID NO: 26, and the LCDR3 comprises the amino acid sequence as set forth in SEQ ID NO: 53;
c) the LCDR1 comprises the amino acid sequence as set forth in SEQ ID NO: 52, the LCDR2 comprises the amino acid sequence as set forth in SEQ ID NO: 26, and the LCDR3 comprises the amino acid sequence as set forth in SEQ ID NO: 67; or,
d) the LCDR1 comprises the amino acid sequence as set forth in SEQ ID NO: 15, the LCDR2 comprises the amino acid sequence as set forth in SEQ ID NO: 16, and the LCDR3 comprises the amino acid sequence as set forth in SEQ ID NO: 17.

In some embodiments, the antigen-binding protein comprises HCDR1, which comprises the amino acid sequence as set forth in SEQ ID NO: 70.

In some embodiments, the HCDR1 comprises the amino acid sequence as set forth in any one of SEQ ID NOs: 20, 35, 56 and 10.

In some embodiments, the antigen-binding protein comprises HCDR2, which comprises the amino acid sequence as set forth in SEQ ID NO: 71.

In some embodiments, the HCDR2 comprises the amino acid sequence as set forth in any one of SEQ ID NOs: 21, 36, 42, 48 and 11.

In some embodiments, the antigen-binding protein comprises HCDR3, which comprises the amino acid sequence as set forth in SEQ ID NO: 70.

In some embodiments, the HCDR3 comprises the amino acid sequence as set forth in any one of SEQ ID NOs: 22, 30, 37, 43, 49, 57, 62 and 12.

In some embodiments, the antigen-binding protein comprises HCDR1, HCDR2, and HCDR3, wherein
a) the HCDR1 comprises the amino acid sequence as set forth in SEQ ID NO: 20, the HCDR2 comprises the amino acid sequence as set forth in SEQ ID NO: 21, and the HCDR3 comprises the amino acid sequence as set forth in SEQ ID NO: 22;
b) the HCDR1 comprises the amino acid sequence as set forth in SEQ ID NO: 20, the HCDR2 comprises the amino acid sequence as set forth in SEQ ID NO: 21, and the HCDR3 comprises the amino acid sequence as set forth in SEQ ID NO: 30;
c) the HCDR1 comprises the amino acid sequence as set forth in SEQ ID NO: 35, the HCDR2 comprises the amino acid sequence as set forth in SEQ ID NO: 36, and the HCDR3 comprises the amino acid sequence as set forth in SEQ ID NO: 37;
d) the HCDR1 comprises the amino acid sequence as set forth in SEQ ID NO: 20, the HCDR2 comprises the amino acid sequence as set forth in SEQ ID NO: 42, and the HCDR3 comprises the amino acid sequence as set forth in SEQ ID NO: 43;
e) the HCDR1 comprises the amino acid sequence as set forth in SEQ ID NO: 20, the HCDR2 comprises the amino acid sequence as set forth in SEQ ID NO: 48, and the HCDR3 comprises the amino acid sequence as set forth in SEQ ID NO: 49;
f) the HCDR1 comprises the amino acid sequence as set forth in SEQ ID NO: 56, the HCDR2 comprises the amino acid sequence as set forth in SEQ ID NO: 36, and the HCDR3 comprises the amino acid sequence as set forth in SEQ ID NO: 57;
g) the HCDR1 comprises the amino acid sequence as set forth in SEQ ID NO: 35, the HCDR2 comprises the amino acid sequence as set forth in SEQ ID NO: 36, and the HCDR3 comprises the amino acid sequence as set forth in SEQ ID NO: 62;
h) the HCDR1 comprises the amino acid sequence as set forth in SEQ ID NO: 35, the HCDR2 comprises the amino acid sequence as set forth in SEQ ID NO: 42, and the HCDR3 comprises the amino acid sequence as set forth in SEQ ID NO: 37;
i) the HCDR1 comprises the amino acid sequence as set forth in SEQ ID NO: 56, the HCDR2 comprises the amino acid sequence as set forth in SEQ ID NO: 36, and the HCDR3 comprises the amino acid sequence as set forth in SEQ ID NO: 57; or
j) the HCDR1 comprises the amino acid sequence as set forth in SEQ ID NO: 10, the HCDR2 comprises the amino acid sequence as set forth in SEQ ID NO: 11, and the HCDR3 comprises the amino acid sequence as set forth in SEQ ID NO: 12.

In some embodiments, the antigen-binding protein comprises a heavy chain variable region VH, which comprises the amino acid sequence as set forth in any one of SEQ ID NOs: 13, 23, 31, 38, 44, 50, 58, 63 and 65.

In some embodiments, the antigen-binding protein comprises a light chain variable region VL, which comprises the amino acid sequence as set forth in any one of SEQ ID NOs: 18, 28, 33, 40, 46, 54, 60 and 68.

In some embodiments, the antigen-binding protein comprises a heavy chain variable region VH and a light chain variable region VL, wherein:
a) the VH comprises the amino acid sequence as set forth in SEQ ID NO: 23 and the VL comprises the amino acid sequence as set forth in SEQ ID NO: 28;
b) the VH comprises the amino acid sequence as set forth in SEQ ID NO: 31 and the VL comprises the amino acid sequence as set forth in SEQ ID NO: 33;
c) the VH comprises the amino acid sequence as set forth in SEQ ID NO: 38 and the VL comprises the amino acid sequence as set forth in SEQ ID NO: 40;
d) the VH comprises the amino acid sequence as set forth in SEQ ID NO: 44 and the VL comprises the amino acid sequence as set forth in SEQ ID NO: 46;
e) the VH comprises the amino acid sequence as set forth in SEQ ID NO: 50 and the VL comprises the amino acid sequence as set forth in SEQ ID NO: 54;
f) the VH comprises the amino acid sequence as set forth in SEQ ID NO: 58 and the VL comprises the amino acid sequence as set forth in SEQ ID NO: 60;
g) the VH comprises the amino acid sequence as set forth in SEQ ID NO: 63 and the VL comprises the amino acid sequence as set forth in SEQ ID NO: 60;
h) the VH comprises the amino acid sequence as set forth in SEQ ID NO: 65 and the VL comprises the amino acid sequence as set forth in SEQ ID NO: 54;
i) the VH comprises the amino acid sequence as set forth in SEQ ID NO: 58 and the VL comprises the amino acid sequence as set forth in SEQ ID NO: 68; or
j) the VH comprises the amino acid sequence as set forth in SEQ ID NO: 13, and the VL comprises the amino acid sequence as set forth in SEQ ID NO: 18.

In some embodiments, the antigen-binding protein comprises a heavy chain constant region, which comprises an IgG-derived constant region.

In some embodiments, the heavy chain constant region comprises a constant region derived from a protein selected from the group consisting of: IgG1, IgG2, IgG3 and IgG4.

In some embodiments, the antigen-binding protein comprises a light chain constant region, which comprises an Igκ-derived constant region or an Igλ-derived constant region.

In some embodiments, the light chain constant region comprises a human Igκ-derived constant region.

In some embodiments, the antigen-binding protein comprises an antibody or an antigen-binding fragment thereof.

In some embodiments, the antigen-binding fragment is selected from the group consisting of: Fab, Fab′, F(ab)2, an Fv fragment, F(ab′)2, scFv, di-scFv, VHH and/or dAb.

In some embodiments, the antibody is selected from the group consisting of: a monoclonal antibody, a murine antibody and a chimeric antibody.

In some embodiments, the detection module comprises an instrument capable of detecting a content of the DNA damage-inducible transcript 4-like (DDIT4L) intron retention spliced product (DIR), and/or of the nucleic acid encoding the DIR.

In some embodiments, the detection module comprises a substance and/or an instrument capable of detecting a content of a marker associated with the cognitive impairment and/or the neurodegenerative disease.

In some embodiments, the marker associated with the cognitive impairment and/or the neurodegenerative disease comprises AD7C-NTP, pTau-181, pTau-217, Aβ40 and/or Aβ42.

In some embodiments, the detection module outputs a content value of the DIR and/or of the nucleic acid encoding the DIR.

In some embodiments, the system comprises a determination module capable of determining a diagnosis result and/or a disease progression result of said subject based on the content of said DIR and/or of said nucleic acid encoding said DIR as well as said normal control value and/or said early control value; the normal control value comprises a content of the DIR and/or of the nucleic acid encoding the DIR in a normal subject; and wherein the early control value comprises a previously measured content of the DIR and/or of the nucleic acid encoding the DIR in the same subject.

In some embodiments, the normal subject does not suffer from the cognitive impairment and/or the neurodegenerative disease.

In some embodiments, when the content of the DIR and/or of the nucleic acid encoding the DIR in the sample of the subject is significantly higher than the normal control value, the determination module diagnoses the subject with the cognitive impairment and/or the neurodegenerative disease.

In some embodiments, the subject has been diagnosed with the cognitive impairment and/or the neurodegenerative disease.

In some embodiments, when the content of the DIR and/or of the nucleic acid encoding the DIR in the sample of the subject is significantly higher than the early control value, the determination module diagnoses the subject with aggravated progression of the cognitive impairment and/or of the neurodegenerative disease.

In some embodiments, the determination module outputs a diagnosis result of the subject.

In some embodiments, the system comprises a display module capable of displaying the diagnosis result of the determination module.

In some embodiments, the system comprises a storage module capable of storing the content value output by the detection module, and/or the diagnosis result output by the determination module.

In another aspect, the present application provides a medicament screening method, comprising the step of: detecting changes in a content of DIR and/or of a nucleic acid encoding the DIR in a subject after administration of a candidate medicament, wherein the medicament is for use in prevention, treatment and/or alleviation of cognitive impairment and/or a neurodegenerative disease.

In some embodiments, the DIR comprises the amino acid sequence as set forth in SEQ ID NO: 1.

In some embodiments, the nucleic acid encoding the DIR comprises a nucleotide sequence as set forth in SEQ ID NO: 8.

In some embodiments, the nucleic acid encoding the DIR comprises a nucleotide sequence as set forth in SEQ ID NO: 93.

In some embodiments, the cognitive impairment comprises cognitive impairment caused by normal aging, Lewy body dementia (LBD), frontotemporal dementia and/or vascular dementia.

In some embodiments, the cognitive impairment-inducing diseases comprise Alzheimer's disease, multi-infarct type, Parkinson's disease, AIDS and/or Creutzfeldt-Jakob disease (CJD).

In some embodiments, the cognitive impairment comprises mild cognitive impairment (MCI), intermediate cognitive impairment and late cognitive impairment.

In some embodiments, the cognitive impairment comprises multi-domain amnestic MCI (aMCI-m).

In some embodiments, the neurodegenerative disease comprises an acute neurodegenerative disease and a chronic neurodegenerative disease.

In some embodiments, the neurodegenerative disease comprises a neurodegenerative disease caused by neuronal death and glial cell homeostasis, a neurodegenerative disease caused by aging, a neurodegenerative disease caused by affected CNS cell function, a neurodegenerative disease caused by abnormal intercellular communication and/or a neurodegenerative disease caused by impaired cell mobility.

In some embodiments, the neurodegenerative disease comprises Alzheimer's disease, Parkinson's disease, multiple sclerosis (MS), amyotrophic lateral sclerosis (ALS) and/or Huntington's disease (HD).

In some embodiments, the neurodegenerative disease comprises presenile dementia.

In some embodiments, the neurodegenerative disease comprises early presenile dementia, intermediate presenile dementia and/or late presenile dementia.

In some embodiments, when the content of the DIR and/or of the nucleic acid encoding the DIR in the subject is reduced due to administration of the candidate medicament, the candidate medicament has a therapeutic effect.

In some embodiments, the administration comprises injection.

In some embodiments, the medicament comprises a small molecule medicament and/or a bio-macromolecular medicament.

In the first aspect of the present application, there is provided application of a substance for detecting an intron retention spliced product (DIR) from a sample of a subject for encoding a DNA damage-inducible transcript 4-like (DDIT4L) in preparation of a product (e.g., a kit) for diagnosis of presenile dementia and mild cognitive impairment and/or evaluation of progression (e.g., grading or staging) of the presenile dementia in the subject.

In some embodiments, the subject is selected from: humans, non-human primates, rodents (such as rats, mice, and guinea pigs), dogs, cats, horses, cattle, and sheep.

In some embodiments, the subject does not suffer from cancer.

In some embodiments, the subject does not suffer from a malignant tumor.

In some embodiments, the DIR has a sequence selected from the group consisting of: a nucleotide sequence (mRNA) of SEQ ID NO: 2; an encoding nucleotide molecule of a nucleotide sequence as set forth in SEQ ID NO: 3, corresponding to a DIR peptide; or a DIR peptide having the amino acid sequence as set forth in SEQ ID NO: 4; a variant derived from the above sequences by substitution, deletion and/or addition of one or more nucleotides and/or amino acids; and a sequence having a high degree of homology (e.g., higher than 80%, 85%, 90%, 95%, 96%, 97%, 98%, 99%, 99.5% sequence homology) to the above sequences.

In some embodiments, the presenile dementia comprises an early stage (an amnesia stage), an intermediate stage (a neurological disorder stage) and a late stage (a severe dementia stage).

In some embodiments, the substance is selected from a reagent, an apparatus, or their combination for detecting the DIR.

In some embodiments, the substance is the one used to detect the DIR at the gene level and/or at the protein level. For example, the substance is the one used in one or more detection techniques or methods selected from the group consisting of: immunohistochemistry (such as immunofluorescence analysis, chemiluminescence, reverse enzyme-linked immunosorbent assay, and an immunocolloidal gold method), Western blotting, Northern blotting, PCR, a biochip method, and a protein chip method.

In some embodiments, the substance is selected from: a substance specific to the DIR, for example, an antibody (e.g., a monoclonal antibody) of the DIR; a probe, gene chip, PCR primer, or gRNA specific to the spliced product, etc.

In some embodiments, the sample is selected from: a blood sample, such as whole blood, serum, and plasma; a tissue or cell sample; and cerebrospinal fluid.

In some embodiments, the blood sample is fresh blood or cryopreserved blood.

In some embodiments, an increase in the DIR level in the sample compared to a normal control value indicates that the subject suffers from presenile dementia or mild cognitive impairment.

In some embodiments, an increase in the DIR level in the sample compared to a mild cognitive impairment control or an early control level in a subject who has been diagnosed with presenile dementia indicates some development (e.g., a higher grade or stage) of the presenile dementia in the subject.

In some embodiments, an increase in the DIR level in the sample compared to a low-grade or low-stage control value indicates some development (e.g., a higher grade or stage) of the presenile dementia in the subject.

In another aspect, the present application provides a diagnostic kit, comprising a substance as described in the present application capable of detecting a DNA damage-inducible transcript 4-like (DDIT4L) intron retention spliced product (DIR), and/or a nucleic acid encoding the DIR.

For example, the present application provides a product (e.g., kit) for diagnosis of presenile dementia and mild cognitive impairment, and/or for evaluation of progression (e.g., a grade or stage) of the presenile dementia, comprising:
(i) a substance for detecting DIR in a sample from a subject; and
(ii) optionally, other substances for diagnosis of the presenile dementia and mild cognitive impairment, and/or for evaluation of progression (e.g., a grade or stage) of the presenile dementia, for example, a substance for detecting an existing marker for the presenile dementia.

In some embodiments, the substance in (i) is selected from one or more of the group consisting of:
a reagent, an apparatus or their combination for detecting the DIR;
a substance for detecting the DIR at the gene level and/or protein level, such as a substance for use in one or more detection techniques or methods selected from the group consisting of: immunohistochemistry (e.g., immunofluorescence analysis, chemiluminescence, reverse enzyme-linked immunosorbent assay, an immunocolloidal gold method), Western blotting, Northern blotting, PCR, and a biochip method; or
a substance specific to the DIR, for example, an antibody (preferably, a monoclonal antibody) of the substance; a probe, gene chip, PCR primer, or gRNA specific to the spliced product.

In some embodiments, other substances in (ii) are selected from: a substance for detecting one or more other markers for presenile dementia, for example: AD7C-NTP, pTau-181, pTau-217, Aβ40 and Aβ42; and/or, a reagent for imaging of brain amyloid protein; and/or, a reagent for diagnosis of the imaging of the subject's brain.

In some aspects of the present application, there is provided a method for screening of a medicament for treatment of presenile dementia. The method comprises: detecting an effect of a candidate medicament on the DIR level in a subject, wherein after the use of the candidate medicament, the decreased level indicates that the candidate medicament has a therapeutic effect on the presenile dementia.

In some embodiments, the detecting is performed using the product as described in the present application.

In some aspects of the present application, there is provided a method for diagnosis of presenile dementia and mild cognitive impairment, and/or for evaluation of progression of the cognitive impairment. The method comprises: detecting a DIR level in a sample from the subject, and accordingly performing diagnosis of presenile dementia and/or evaluation of progression (e.g., grading or staging) of the presenile dementia on the subject.

In some embodiments, the detecting is performed using the product as described in the present application.

In some embodiments, an increase in the DIR level in the sample compared with the control value indicates that the subject suffers from presenile dementia; or, an increase in the DIR level in the sample compared with a low-grade or low-stage control indicates some development (e.g., a higher grade or stage) of the presenile dementia in the subject.

In some aspects of the present application, there is also provided a system corresponding to the method of the present application. The system comprises the following devices:
(a) a device for collecting and/or receiving data on a DIR level in a sample; and
(b) a device for analyzing the data for diagnosis of presenile dementia and mild cognitive impairment and/or for evaluation of progression (e.g., grading or staging) of the presenile dementia in the subject, wherein an increase in the DIR level in the sample compared to the normal control value indicates that the subject suffers from the presenile dementia or mild cognitive impairment; or, an increase in the DIR level in the sample compared to a mild cognitive impairment control or an early control level in a subject who has been diagnosed with presenile dementia indicates some development (e.g., a higher grade or stage) of the presenile dementia in the subject.

In some embodiments, the system further comprises (a′) a device for collecting and/or receiving presenile dementia diagnosis and/or data on other clinical indicators or basic characteristics of the development of the presenile dementia in a sample.

In some embodiments, the device described in (b) is used to combine and analyze DIR level data with data on other clinical indicators or basic characteristics of presenile dementia diagnosis and/or development progression of presenile dementia, in order to perform prognosis evaluation on the presenile dementia diagnosis and/or the development progression of presenile dementia of the subject.

Or, in some embodiments, the system further comprises a device for analyzing presenile dementia diagnosis and/or data on other clinical indicators or basic characteristics of the development progression of presenile dementia; and/or a device for combining and analyzing DIR level data with the data on other clinical indicators or basic characteristics of presenile dementia diagnosis and/or the development progression of presenile dementia, in order to evaluate the presenile dementia diagnosis and/or the development progression of presenile dementia in the subject.

In some embodiments, the system further comprises one or more devices selected from the group consisting of: a device for inputting and/or outputting and/or storing data; a device for outputting and/or storing analysis results, for example, a device for uploading and/or storing the analysis results to a cloud database or its corresponding cloud database device; and a device for performing intelligent analysis and recommending a therapeutic scheme based on prognosis evaluation results.

It should be understood that those skilled in the art may add other conventional devices or programs to the system as needed.

Other aspects and advantages of the present application may be readily perceived by those skilled in the art from the detailed description below. The detailed description below only illustrates and describes the exemplary embodiments of the present application. As would be appreciated by those skilled in the art, the content of the present application allows those killed in the art to change the specific embodiments disclosed without departing from the spirit and scope involved in the present application. Accordingly, the accompanying drawings and the description in the specification of the present application are merely for an exemplary but not restrictive purpose.

### BRIEF DESCRIPTION OF THE DRAWINGS

The specific features of the invention involved in the present application are listed in the appended claims. The characteristics and advantages of the invention involved in the present application may be better understood by referring to the exemplary embodiments and the accompanying drawings described in detail below. A brief description of the drawings is as follows:

FIGS. 1A-1B show the splicing of a DDIT4L mRNA precursor and the secretion of a DIR protein from transfected cells into a medium. In the figures: FIG. 1A shows the splicing of DDIT4L, in which the length of the mRNA obtained by normal shearing of an intron is 582 nt (SEQ ID NO: 95) and the length of an RNA encoding the DIR in the mRNA obtained by abnormal splicing of intron retention is 255 nt (SEQ ID NO: 8); and FIG. 1B shows the secretion of the DIR from cells into a medium, in which a pCMV-flag-DIR plasmid is transfected into HEK293 cells and cultured for 48 h, followed by the observation that a secreted DIR protein is detectable in the medium. DIR protein bands are visible in DIR plasmid lane of the lysate, which are 12 kDa in size.

FIGS. 2A-2B show changes in the content of DIR protein in the blood of AD patients and of patients with a variety of other diseases. FIG. 2A shows changes in the content of DIR in the blood of AD patients and of patients with a variety of other diseases by Western assay; and the plasma protein, the light chain of immunoglobulins, in the figure serves as an internal reference. FIG. 2B shows changes in the content of DIR in the blood of AD patients, MCI patients, and non-AD/MCI patients by ELISA assay. In the figures, *** represents P<0.001, and * represents P<0.05.

FIG. 3 shows a standard curve of a standard DIR protein detected by ELISA.

FIG. 4 shows a ROC curve of a clinical sample detected by ELISA.

FIG. 5 shows changes in the content of DIR in the blood of patients with different classes of MCI, compared with normal follow-up controls.

FIG. 6 shows changes in the content of DIR and other indicators in the blood of patients with different classes of MCI and AD, compared with normal controls.

FIG. 7 shows the correlation of DIR expression levels in plasma and cerebrospinal fluid.

FIG. 8 shows the detection results on the binding activity of the DIR antibody described in the present application.

FIGS. 9A-9B show the correlation of DIR RNA levels in AD patients. FIG. 9A shows the increased expression of DIR from previous brain RNA-seq data of AD patients and old people (GSE153873 and GSE159699) using IRfinder. **, p<0.01. FIG. 9B shows the increased expression of DIR mRNA in the AD patients' hippocampi as compared with that of non-AD old people. ***, p<0.001.

FIG. 10 shows the species difference of human DDIT4L and DIR protein sequences, and the human DIR knock-in sequence in the mouse, including the comparison among human, chimpanzee and mouse DDIT4L sequences and the amino acid sequences of constructed DIR expressed in the DIR-KI mice.

FIGS. 11A-11H show the generation of homozygous human DIR knock-in mice. FIG. 11A shows that the DIR knock-in (KI) mouse was constructed via CRISPR/Cas9 assays. To ensure the correct and intact expression of human DIR, the human DIR sequence encoding the last 64 AAs (the green block showing the 21-30 AAs of human DDIT4L, the red one for the 31-84 AAs of the DDIT4L intron translated region) was inserted behind the mouse DDIT4L genome that translated 1-20 AAs. FIG. 11B shows that the genotyping of newborn mice confirmed the accurate sequence insertion of DIR (WT: wild-type, HE: heterozygote, HO: homozygote). FIG. 11C shows that the designed primers identified the human DIR mRNA in the HO mice. FIG. 11D shows the DIR protein was expressed in the hippocampi of HO mice. FIG. 11E shows that the DIR was expressed in the hippocampal dentate gyrus neurons of 6-month-old HO mice. Scale bar=100 μm. FIG. 11F shows that the DIR expression in hippocampi was increased in the HO mice exposed to the hypoxia condition (8% O₂, 8 h), the DIR expression was apparently increased in the ipsilateral hippocampus in MCAO hypoxic-ischemic brain injury model of HO mice. FIG. 11G and FIG. 11H shows that the specific primers confirmed the mouse intrinsic intron of DDIT4L (mDIR) in WT mice. The mDDIT4L expression have no change between WT and HO mice. Data shown are mean±S.E.M. Two-tailed unpaired t-test.

FIGS. 12A-12F show the normal growth and motor ability of DIR-KI mice and the antibody of DIR verification in the hippocampi of DIR-KI mice. FIG. 12A shows the body weight of the homozygous DIR-KI mice was not different from that of WT mice. FIG. 12B shows that no difference in the moving distance and velocity between WT mice and the homozygous DIR-KI mice in the open field test. FIG. 12C shows that an immunoreactive bands (˜12 kDa, arrowheads) detected with the antibody against DIR, but not by the pre-absorbed antibody in the hippocampi tissue of DIR-KI mice (HO). FIG. 12D shows the primary immunoblotting lanes of DIR in the hippocampi tissue of the DIR-KI mice (HO) shown in FIG. 11D. WT, wild-type mice. FIG. 12E shows that in HEK293 cells, transfected Flag-DDIT4L (2 μg), Flag-DIR-1 (1 μg), Flag-DIR-2 (2 μg), Flag-DIR-3 (3 μg), then the antibodies against C-terminal of DDIT4L, DIR-Exon, DIR-Intron, DIR-Intron (N27aa, 27aa of Intron N-terminal) and Flag. The arrowhead shown special band for DIR, the #signal shown special band for DDIT4L. FIG. 12F shows the antigen absorption of DIR antibody abolished the DIR staining in homozygous DIR-KI mice. Scale bar=50 m. Data shown are mean±S.E.M. Two-tailed unpaired t-test.

FIGS. 13A-13D show the primary lanes for FIG. 11F and FIG. 11H. FIG. 13A shows the primary immunoblot lanes of DIR shown in the left part of FIG. 11F. FIG. 13B shows the primary immunoblot lanes of DIR shown in the right part of FIG. 11F. FIG. 13C shows the immunostaining shown the increased expression of DIR in the ipsilateral hippocampus in MCAO hypoxic-ischemic brain injury model of homozygous DIR-KI mice (n=4). Scale bar=100 μm. FIG. 13D shows the primary immunoblot lanes of mDDIT4L shown in the right part of FIG. 11H.

FIGS. 14A-14C show the detection of DIR and mDIR. FIG. 14A shows the immunoblotting shows that flag antibody could not detect flag-mDIR in the lysate of HEK293 cells transfecting flag-mDIR plasmid. FIG. 14B shows that the mDIR was not detected in the hippocampi of normal controls and MCAO model of WT mice by two different antibodies. FIG. 14C shows that the flag-mDIR was not detected after the immunoprecipitation with flag antibody while flag-DIR was present.

FIGS. 15A-15G show the AD-like pathological features in homozygous DIR-KI mice. FIG. 15A shows that the expression of AD- or p-Tau was upregulated in the hippocampal dentate gyrus in the 6-month-old homozygous DIR-KI (HO) mice, compared with wildtype (WT) mice. Scale bars=100 μm. *, p<0.05. **, p<0.01. FIG. 15B shows that the HO mice increased the escape latency following a 5-day training period compared with that of WT mice in the Morris water maze test. In the probe test to assess the spatial memory, WT mice had more times for crossing platform and spent less time to find the platform, compared with the HO mice. **, p<0.01. FIG. 15C shows that spontaneous excitatory postsynaptic current (sEPSC) of hippocampal neurons was inhibited by the DIR-Intron (10 μM) in the brain slices prepared from WT mice. FIG. 15D shows that the amplitude of sEPSC in hippocampal neurons was suppressed by the DIR-Intron in a dose-dependent manner. **, p<0.01. ***, p<0.001. FIG. 15E shows that the frequency of sEPSC in hippocampal neurons was dose-dependently reduced by the DIR-Intron. *, p<0.05. n.s., no significance. FIG. 15F shows that the long-term potentiation (LTP) of field EPSP recorded in the hippocampal CA1 region of the HO mice was apparently decreased, as compared with that of WT mice. ***, p<0.001. FIG. 15G shows that the capacity of novel object recognition was reduced in the group treated with the DIR-Intron via the catheter implanted into the hippocampal CA1 region, delivering the DIR-Intron peptide locally through the subcutaneous minipumps (200 ng per mouse). O, old object; N, new object. **, p<0.01. Data shown are mean±S.E.M. Two-tailed unpaired t-test (A, B, D, E, G) or two-way ANOVA test followed by Bonferroni correction (B, F).

FIGS. 16A-16C show the Aβ and p-Tau expression in 3 and 9-month-old DIR-KI mice, and the DDIT4L expression in 3-month-old DIR-KI mice. FIG. 16A shows that the expression of Aβ and p-Tau was upregulated in the hippocampal dentate gyrus in the 3-month-old homozygous DIR-KI mice. Scale bars=100 μm. *, p<0.05. **, p<0.01. FIG. 16B shows that the expression of Aβ or p-Tau was upregulated in the hippocampal dentate gyrus in the 9-month-old homozygous DIR-KI mice. Scale bars=100 μm. **, p<0.01. FIG. 16C shows that the expression of DDIT4L was not changed in the hippocampal dentate gyrus in the WT and homozygous DIR-KI mice. Scale bars=100 μm. n.s., no significance. Data shown are mean±S.E.M. Two-tailed unpaired t-test.

FIGS. 17A-17C show the DIR colocalization of Aβ and p-Tau. FIG. 17A shows that DIR and Aβ were colocalized in the hippocampi of 6-month-old DIR-KI mice. Scale bars=100 μm. FIG. 17B shows that DIR and p-Tau were colocalized in the hippocampi of 6-month-old DIR-KI mice. Scale bars=100 m. FIG. 17C shows the quantificational analysis on the colocalization of DIR, Aβ and p-Tau in the hippocampi of 6-month-old DIR-KI mice. ***, p<0.001. Data shown are mean±S.E.M. Two-tailed unpaired t-test.

FIGS. 18A-18D show the verification of Aβ and p-Tau antibodies. FIG. 18A shows that the antigen absorption of Aβ antibody (6E10) abolished the immunostaining in the hippocampi of 6-month-old DIR-KI mice. Scale bars=100 m. FIG. 18B shows that the antigen absorption of p-Tau antibody abolished the immunostaining in the hippocampi of 6-month-old DIR-KI mice. Scale bars=100 μm. FIG. 18C shows that the Aβ antibody (6E10) detected Aβ in the hippocampal neurons of 6-month-old 5×FAD mice. Scale bars=100 μm. FIG. 18D shows that the p-Tau antibody detected AT8-positive neurons in the hippocampi of 6-month-old 3×Tg mice. Scale bars=100 μm.

FIGS. 19A-19D show the working memory and novel object recognition of DIR-KI mice. FIG. 19A shows in the Y maze alternative test that the working memory was impaired in the homozygous DIR-KI mice (HO), as compared with that of wild-type mice (WT). *, p<0.05. FIG. 19B shows that the capacity of novel object recognition was reduced in the homozygous DIR-KI mice compared with that of WT mice. O, old object; N, new object. **, p<0.01. FIG. 19C shows that the amplitude and frequency of sEPSC of hippocampal neurons were not altered by applying DIR-Exon (10 μM) in the brain slices prepared from WT mice. n.s., no significance. FIG. 19D shows the amplitude of sEPSC in hippocampal neurons increased by DIR (1 μM). ***, p<0.001. n.s., no significance. Data shown are mean±S.E.M. Two-tailed unpaired t-test.

FIGS. 20A-20E show the improved working memory and novel object recognition in DIR-KI mice treated with DIR mAb, diffused into the brain tissues; DIR antibody treatment reduced DIR bound to GluA1 or gelsolin in hippocampus of HO mice. FIG. 20A shows in the Y maze alternative test that the working memory could be partially reversed by the DIR mAb treatment in the DIR-KI mice, as compared with the mice treated with IgG. *, p<0.05. FIG. 20B shows that the capacity of novel object recognition was partially reversed by the DIR mAb treatment in the DIR-KI mice, as compared with the mice treated with IgG. **, p<0.01. FIG. 20C shows that two-photon imaging of mouse brain tissue after intravenous injection of FITC conjugated DIR antibody. Dextran-Texas Red showed that FITC-tagged DIR antibody diffused from blood vessels into the brain tissue (n=3 mice). ***, p<0.01. FIG. 20D shows in the PLA assay that DIR antibody treatment could reduce DIR bound to gelsolin in the hippocampus of HO mice. Scale bars=50 μm. *, p<0.05. FIG. 20E shows in the PLA assay that DIR antibody treatment could reduce DIR bound to GluA1 in the hippocampus of HO mice. Scale bars=50 μm. *, p<0.05. Data shown are mean±S.E.M. Two-tailed unpaired t-test.

FIGS. 21A-21E show the reversing AD-like pathological features by DIR mAb. FIG. 21A shows that the DIR antibody by intraperitoneal injection could cross the blood brain barrier (BBB) after 24 h. ***, p<0.001. FIG. 21B shows the representative trial traces of individual mice on probe test day. In the probe test to assess spatial memory, the mice treated with the DIR mAb had more times for crossing platform and spent less time to find platform, compared with the mice treated with IgG. *, p<0.05. **, p<0.01. FIG. 21C shows that the expression of Aβ- and p-Tau was apparently decreased in the hippocampal dentate gyrus in the 6-month-old HO mice treated with the DIR mAb. Scale bars=100 μm. *, p<0.05. **, p<0.01. FIG. 21D shows that the LTP in the HO mice was apparently increased after the DIR mAb treatment, as compared with IgG treatment (n=5 mice per group). **, p<0.01. FIG. 21E shows that the spontaneous excitatory postsynaptic current (sEPSC) of hippocampal neurons was increased after the DIR mAb treatment, as compared with IgG treatment. The amplitude, not frequency of sEPSC in hippocampal neurons was apparently increased after the DIR mAb treatment, as compared with IgG treatment. **, p<0.01. n.s., no significance. Data shown are mean±S.E.M. Two-tailed unpaired t-test (A, B, C, E) or two-way ANOVA test followed by Bonferroni correction (D).

### DETAILED DESCRIPTION OF THE PREFERRED EMBODIMENTS

The embodiments of the invention of the present application will be illustrated by specific examples below. Those familiar with this technology may easily understand other advantages and effects of the invention of the present application from the content disclosed in the specification.

### TERMS & DEFINITIONS

The term “DDIT4L” generally refers to DNA damage-inducible transcript 4-like. It can also be called REDD2/RTP801L. Studies have found that DDIT4L can be associated with cardiac dysfunction. It can also be used to treat gliomas. The accession number of a human DDIT4L gene in GenBank is 115265; the accession number of a human DDIT4L protein in GenBank is NP_660287.1.

The term “QDLIR” generally refers to the intron retention spliced product of DDIT4L. In the present application, the amino acid sequence of the QDLIR may be as set forth in SEQ ID NO: 1. In the present application, QDLIR can be used interchangeably with DIR, and it can be an intron retention form produced by abnormal splicing during expression of the human DDIT4L gene. In the present application, a gene encoding the DIR may be referred to as a DIR gene. DIR may comprise the amino acid sequence encoded by a first exon (with the amino acid sequence as set forth in SEQ ID NO. 2) and the amino acid sequence encoded by a retained intron (with the amino acid sequence as set forth in SEQ ID NO. 3). Here, the amino acid sequence (IR) encoded by the retained intron may be divided into two parts, namely, IR-I (i.e. DIR-I, with the amino acid sequence as set forth in SEQ ID NO. 4) consisting of the first 27 amino acids from the N-terminus, and DIR-II (i.e., DIR-II, with the amino acid sequence as set forth in SEQ ID NO. 5) consisting of the last 27 amino acids. The term “expression level” generally refers to a protein, RNA or mRNA level of a specific related gene. Any method known in the art can be used to determine the expression level of a specific related gene (e.g., a human DDIT4L gene). In the present application, the “expression” generally refers to the process of transforming the information encoded by a gene into a structure that exists in and operates in a cell. For example, reverse transcription and amplification analysis (e.g., PCR, ligation-dependent RT-PCR or quantitative RT-PCT), hybridization analysis, Northern blotting, dot blotting, in situ hybridization, gel electrophoresis, capillary electrophoresis, column chromatography, Western blotting, immunohistochemistry, immunostaining, or mass spectrometry can be included. A biological sample or a protein/nucleic acid isolated from the sample can be analyzed directly.

The term “neurodegenerative disease” generally refers to dementia or other cognitive impairment resulting from the gradual loss of neuronal structures and functions, including neuronal death and glial cell homeostasis. In some cases, age (e.g., Alzheimer's disease (AD), Parkinson's disease (PD)) or genetic mutations (e.g., Huntington's disease, early-onset AD or PD, amyotrophic Lateral sclerosis (ALS)) that affect CNS cell function may induce the neurodegenerative disease. The neurodegenerative disease may have changes and/or disorders selected from the group consisting of: protein misfolding and aggregation; neuroinflammation (e.g., CNS inflammation occurring in response to signal stimuli such as toxic stimuli (e.g., protein aggregation), infection, traumatic injury, or autoimmune); changes in cell signaling; acquired senescence/cell death (e.g., disrupted apoptotic signaling, mitochondrial dysfunction, impaired autophagy, and activation of necrosomes by stress/inflammation); and motor cell damage and epigenetic changes.

The term “Alzheimer's disease” generally refers to presenile dementia or senile dementia, which is a neurodegenerative disease that develops slowly and gets worse over time. The most common early symptom is loss of short-term memory (having difficulty in remembering recent events). As the disease progresses, at least one of the following symptoms may gradually appear: language disorder, disorientation (for example, getting lost easily), emotional lability, loss of motivation, inability to take care of oneself and behavioral problems. The true cause of Alzheimer's disease remains unknown, but its progression may be related to the deposition of fibrillar amyloid protein plaques and tau protein in the brain. At present, there is no therapy that can stop or reverse the course of the disease, but a few methods that may temporarily relieve or ameliorate symptoms.

The term “presenile dementia” is used interchangeably with the term “Alzheimer's disease” in the present application. The presenile dementia comprises early presenile dementia, intermediate presenile dementia and/or late presenile dementia. For example, the learning and memory impairment of the patients with early presenile dementia will become more and more obvious. In some cases, they will develop language disorder, executive impairment, cognitive impairment (agnosia) and/or skill execution impairment (apraxia). For example, the patients with intermediate presenile dementia will lose the ability to live independently and may be unable to carry out most of daily activities (in some cases, they may suffer from anomia, paraphasia, and/or anosognosia). For example, the patients with late presenile dementia may depend on caregivers in the late stage. For example, they may completely lose language competence. For example, they may not be able to feed themselves.

The term “cognitive impairment” generally refers to a disease and condition that is considered to or do involve the progressive loss of neuronal structure and/or function (including neuronal death) or those described above. For example, the characteristics of cognitive impairment may comprise impairment of cognition (e.g., memory, attention, perception, and/or thinking). These impairment may comprise pathogen-induced cognitive dysfunction, for example, HIV-related cognitive dysfunction and Lyme disease-related cognitive dysfunction. Examples of cognitive impairment may comprise Alzheimer's disease, Huntington's disease, Parkinson's disease, amyotrophic lateral sclerosis (ALS), autism, mild cognitive impairment (MCI), stroke, traumatic brain injury (TBI) and/or age-associated memory impairment (AAMI).

The term “mild cognitive impairment (MCI)” generally refers to an intermediate clinical state between normal cognition and cognitive impairment. In some cases, the MCI may comprise cognitive impairment that meets criteria for dementia but is worse than normal aging. MCI is diverse in clinical manifestation, etiology, prognosis, and prevalence. In some cases, MCI may be a pathological stage of Alzheimer's disease. Some forms of cognitive impairment may be considered as early manifestations of the neurodegenerative disease and may eventually lead to dementia. In some cases, the MCI may comprise a subtype selected from the group consisting of: aMCI-s: single-domain amnestic MCI; aMCI-m: multi-domain amnestic MCI; naMCI-s: single-domain non-amnestic MCI; and naMCI-m: multi-domain non-amnestic MCI.

The term “cognitive impairment caused by normal aging” generally refers to cognitive impairment due to normal aging. For example, the cognitive impairment caused by normal aging may be manifested as: memory loss, confusion about the location of familiar places, taking longer time than usual to complete daily tasks, or changes in mood and personality.

The term “Lewy body dementia (LBD)” generally refers to dementia with Lewy bodies. Dementia with Lewy bodies is characterized by abnormal accumulation of proteins into lumps called Lewy bodies. Dementia with Lewy bodies cause a gradual decline in mental ability. People with Lewy body dementia may experience visual hallucinations and changes in alertness and attention. Other influences comprise muscle stiffness, slowness of movement, difficulty in walking and tremors. Patients with Lewy bodies in the brain may also have plaques and tangles associated with Alzheimer's disease.

The term “frontotemporal dementia” generally refers to Pick's disease, which is a rare, progressive disease in which a tau protein affects only the frontal and temporal lobes of the brain. Patients with frontotemporal dementia have difficulty in higher-level reasoning, language expression, speech perception, and memory formation. In patients with frontotemporal dementia, the frontal and temporal lobes of the brain may atrophy over time.

The term “vascular dementia” generally refers to problems with reasoning, judgment, and memory caused by impaired blood flow to the brain. For example, the vascular dementia may comprise dementia caused by risk factors for heart disease and stroke, such as high blood pressure and high cholesterol.

The term “multi-infarct type” generally refers to small noncortical infarcts caused by occlusion of a single perforating branch of a large cerebral artery. The multi-infarct type may be a special type of cerebral infarction, also known as ischemic stroke. The multi-infarct type may be manifested as hemidysesthesia, aphasia, anarthria, slow movement, and clumsiness (especially difficulty with fine movements such as writing).

The term “Parkinson's disease” generally refers to a progressive neurodegenerative disease. The clinical features of Parkinson's disease (PD) may comprise motor symptoms (e.g., tremor, bradykinesia, myotonia, and postural instability), as well as neuropsychiatric and other non-motor manifestations. For example, the non-motor manifestations may comprise cognitive dysfunction and dementia, mood disorders (e.g., depression, anxiety, apathy), and sleep disorders.

The term “Creutzfeldt-Jakob disease (CJD)” generally refers to a type of transmissible spongiform encephalopathy that occurs in humans. CJD is a disease caused by prion infection. CJD patients may manifest paranoid behavior, confusion, loss of appetite and weight, and depression, and a few patients have visual or auditory abnormalities; and in the progressive stage, they manifest progressive neurological deterioration (e.g., sensory abnormalities, language disorder, and aphasia).

The term “multiple sclerosis (MS)” generally refers to a demyelinating neuropathy. Insulating substances (i.e., myelin sheath) on the surfaces of nerve cells in the brain or spinal cord of the MS patients are damaged, and the signal transduction of the nervous system is damaged, which can lead to a series of possible symptoms, affecting the activity, mental ability, and even mental state. These symptoms may comprise diplopia, unilateral visual impairment, myasthenia, dysesthesia, or dystaxia.

The term “amyotrophic lateral sclerosis (ALS)” generally refers to a motor neuron disease, which is a progressive and fatal neurodegenerative disease. A small number of ALS patients may develop frontotemporal lobe dementia. Some ALS patients may experience deterioration in their senses of sight, touch, smell, and taste, and a very small number of ALS patients may develop dementia at the same time.

The term “Huntington's disease (HD)” generally refers to a genetic disease that causes brain cell death. With the progress of this disease, HD patients may experience more obvious incoordination of body movements, and their ability gradually deteriorates until movement becomes difficult and they cannot speak. Then, the mental ability generally declines into dementia.

The term “aging stage” generally refers to a subject's stage of aging. For example, for humans, the aging stage may be over 60 years old, over 70 years old, or over 75 years old; and for mice, the aging stage may be over 10 months old, for example, over 13 months old or over 18 months old. In some cases, a subject in the aging stage may have one or more symptoms of learning deficit, memory impairment, memory deficit, and/or brain dysfunction.

The term “variant” generally refers to a polypeptide comprising the amino acid sequence that differs by at least one amino acid residue from the amino acid sequence of a parent or reference polypeptide (e.g., a wild-type polypeptide). In the present application, the variant may have a high (e.g., at least 80%) homology to the parent or reference polypeptide. The homology may comprise sequence similarity or identity. In the present application, the homology may be determined using standard techniques known in the art (see, for example, Smith and Waterman, “Advances in Applied Mathematics”); and the percentage of identity shared by polynucleotide or polypeptide sequences is determined by direct comparison of sequence information between molecules, and the comparison is performed by sequence alignment to determine the identity using methods known in the art. An example of an algorithm suitable for determining sequence similarity is the BLAST algorithm (see Altschul et al., Journal of Molecular Biology, 215: 403-410 [1990]). Software for BLAST analyzes is publicly available through the National Center for Biotechnology Information (NCBI).

In the present application, the term “CDR”, short for “complementary determining region”, generally refers to a region of an antibody variable domain, and its sequence is highly variable and/or forms a structure-defined ring. For example, the antibody may comprise six CDRs, with three (HCDR1, HCDR2, and HCDR3) in VH and three (LCDR1, LCDR2, and LCDR3) in VL. In some embodiments, a naturally occurring camelid antibody composed of heavy chains only still functions normally and stably in the absence of light chains. The CDR of antibody can be determined by a variety of coding systems, such as CCG, Kabat, Chothia, IMGT, the combination of Kabat/Chothia, etc. These encoding systems are known in the art. See, for example, www.bioinf.org.uk/abs/index.html#kabatnum for details. For example, the amino acid sequence number of the antigen-binding protein can be numbered according to the IMGT numbering scheme (IMGT, the international ImMunoGeneTics information system@imgt.cines.fr; imgt.cines.fr; Lefranc et al., 1999, Nucleic Acids Res. 27: 209-212; Ruiz et al., 2000 Nucleic Acids Res. 28: 219-221; Lefranc et al., 2001, Nucleic Acids Res. 29: 207-209; Lefranc et al., 2003, Nucleic Acids Res. 31: 307-310; Lefranc et al., 2005, DevComp Immunol 29: 185-203). For example, the CDR of the antigen-binding protein can be determined according to the Kabat numbering system (see, for example, Kabat E A &Wu T T (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242).

In the present application, the term “FR” generally refers to a more highly conserved part in the variable domain of an antibody, and it is referred to as a framework region. For example, the variable domains of naturally occurring heavy and light chains may each comprise four FRs, namely four (H-FR1, H-FR2, H-FR3, and H-FR4) in VH, and four (L-FR1, L-FR2, L-FR3, and L-FR4) in VL.

In the present application, the terms “variable domain” and “variable region” are used interchangeably and each generally refer to a portion of an antibody heavy chain and/or light chain. The variable domains of the heavy and light chains may be referred to as “VH” and “VL” respectively (or “VH” and “VL” respectively). These domains are generally the most varied fractions of an antibody (with respect to other antibodies of the same type) and may comprise antigen-binding sites. In the present application, the term “variable” generally refers to the fact that certain segments of the variable domains may differ greatly in sequence between antibodies. A variable domain-mediated antigen may bind and determine the specificity of a specific antibody to its specific antigen. However, the variability may not be evenly distributed across the entire range of variable domain. It may be generally concentrated in three segments called hypervariable regions (CDRs or HVRs) in the variable domains of the light and heavy chains. The more highly conserved part of the variable domain may be referred to as framework region (FR). The variable domains of the naturally occurring heavy and light chains may each comprise four FRs, most of which have a β-pleated configuration and which are linked by three CDRs to form a circular linkage, and to form a fraction of a β-pleated structure in some cases. The CDRs in each chain may be held together closely by FR regions, and the CDRs from another chain jointly promote the formation of the antigen-binding site of the antibody.

In the present application, the term “antibody” generally refers to an immunoglobulin or a fragment or derivative thereof, and encompasses any polypeptide comprising an antigen-binding site, regardless of its production in vitro or in vivo. The term may comprise, but is not limited to, polyclonal, monoclonal, monospecific, multispecific, non-specific, humanized, single-chain, chimeric, synthetic, recombinant, hybrid, mutant and grafted antibodies. Unless otherwise modified by the term “intact” (as in “intact antibody”), for the purposes of the present invention, the term “antibody” may also comprise antibody fragments, such as Fab, F(ab′)2, Fv, scFv, Fd, VHH, dAb, and other antibody fragments that maintain the antigen-binding function (for example, specific binding to human DIR). Generally, such fragments may comprise antigen-binding domains. A basic 4-chain antibody unit may be a heterotetrameric glycoprotein composed of two identical light (L) chains and two identical heavy (H) chains. An IgM antibody may be composed of 5 basic heterotetrameric units and another polypeptide called J chain, and comprise 10 antigen-binding sites; and an IgA antibody may comprise 2-5 basic 4-chain units that may bind to and be polymerized with the J chain to form a multivalent combination. In the case of IgG, the 4-chain unit may be generally of about 150,000 Daltons. Each L chain may be linked to the H chain by a covalent disulfide bond, and two H chains may be linked to each other by one or more disulfide bonds depending on the isotype of the H chains. Each of the H and L chains may also have a regularly spaced intra-chain disulfide bridged bond. Each H chain may have a heavy chain variable domain (VH) at its N-terminus, followed by three constant domains (CH) for each of α and γ chains, or followed by four CH domains for the μ and ε isoforms. Each L chain may have a light chain variable domain (VL) at its N-terminus and a constant domain at the other end. VL may correspond to VH, and light chain constant region (CL) may correspond to a first constant domain (CH1) of the heavy chain. Specific amino acid residues may be considered to form an interface between the variable domains of the light and heavy chains. VH and VL may be paired together to form a single antigen-binding site. An L chain from any vertebrate species may be classified, based on the amino acid sequence of its constant domain, into one of two distinct types, called κ and λ. Immunoglobulins may be classified into different classes or isotypes depending on the amino acid sequences of constant domains of the heavy chains (CH). At present, there are five classes of immunoglobulins, namely, IgA, IgD, IgE, IgG (e.g., IgG1, IgG2, IgG3 and/or IgG4), and IgM, which have heavy chains named α, δ, ε, γ, and μ, respectively.

In the present application, the term “antigen-binding fragment” generally refers to one or more fragments capable of specifically binding to an antigen (for example, DIR). In the present application, the antigen-binding fragment may comprise Fab, Fab′, F(ab)2, a Fv fragment, a F(ab′)2, scFv, di-scFv, VHH and/or dAb.

In the present application, the term “Fab” generally refers to the antigen-binding fragment of an antibody. As described above, an intact antibody may be digested using papain. The antibody digested by the papain produces two identical antigen-binding fragments, namely, a “Fab” fragment, and a residual “Fc” fragment (i.e., an Fc region). The Fab fragment may be composed of a complete L chain, the variable region of a heavy chain and the first constant region (CH1) of the H chain (VH).

In the present application, the term “F(ab)2” generally refers to the antigen-binding fragment of an antibody. For example, F(ab)2 may be formed by linking two Fab fragments.

In the present application, the term “Fab′” generally refers to a monovalent antigen-binding fragment of a human monoclonal antibody, and the fragment is slightly larger than a Fab fragment. For example, a Fab′ fragment may comprise all of the light chains, all of the variable regions of the heavy chain, and all or some of the first and second constant regions of the heavy chain. For example, the Fab′ fragment may further comprise some or all of the 220-330 amino acid residues of the heavy chain.

In the present application, the term “F(ab′)2” generally refers to an antibody fragment produced by pepsin digestion of an intact antibody. The F(ab′)2 fragment comprises two Fab fragments held together by a disulfide bond, and some of hinge regions. The F(ab′)2 fragment have a bivalent antigen-binding activity and is capable of cross-linking an antigen.

In the present application, the term “Fv fragment” generally refers to a monovalent antigen-binding fragment of a human monoclonal antibody, including all or some of the heavy-chain and light-chain variable regions, and lacking the heavy-chain and light-chain constant regions. The heavy-chain and light-chain variable regions comprise, for example, CDRs. For example, an Fv fragment comprises all or some of the amino-terminus variable regions of about 110 amino acids of the heavy and light chains.

In the present application, the term “scFv” generally refers to a fusion protein comprising at least one antibody fragment comprising a light chain variable region and at least one antibody fragment comprising a heavy chain variable region, wherein the light-chain and heavy-chain variable regions are contiguous (e.g., via a synthetic linker such as a short flexible polypeptide linker) and can be expressed as a single chain polypeptide, and wherein the scFv retains the specificity of the intact antibody from which it is derived. Unless otherwise stated, as used in the present application, the scFv may have the VL and VH variable regions described in any order (e.g., relative to the N-terminus and C-terminus of the polypeptide), and the scFv may comprise VL-linker-VH or VH-linker-VL.

In the present application, the term “dAb” generally refers to an antigen-binding fragment consisting of a VH domain or a VL domain. Refer to, for example, Ward et al. (Nature, 1989 Oct. 12; 341(6242): 544-6), and Holt et al., Trends Biotechnol., 2003, 21(11): 484-490.

In the present application, the term “VHH” generally refers to an antibody comprising the antigen-binding variable domain of an antibody comprising a heavy chain (see Vanlandschoot P. et al., 2011, Antiviral Research, 92, 389-407). VHH may also be called nanobody (Nb).

In the present application, the term “monoclonal antibody” generally refers to an antibody molecule product consisting of single molecules. The monoclonal antibody is generally highly specific for a single antigen site. Moreover, unlike conventional polyclonal antibody preparations (which typically have different antibodies for different determinants), each monoclonal antibody can target a single determinant on an antigen. In addition to their specificity, the advantage of monoclonal antibodies lies in that they may be synthesized by hybridoma culture, without being contaminated by other immunoglobulins. The modifier “monoclonal” may indicate the antibody characteristics obtained from a substantially homogeneous population of antibodies, and should not interpreted as requiring the production of antibodies by any particular method. For example, the monoclonal antibodies used in the present application may be prepared in hybridoma cells, or may be prepared by recombinant DNA methods.

In the present application, the term “chimeric antibody” generally refers to an antibody in which the variable region is derived from one species and the constant region is derived from another species. Generally, the variable region is derived from an antibody (“parent antibody”) in an experimental animal such as a rodent, and the constant region is derived from a human antibody, such that the possibility of causing an adverse immune response in an individual human by the resulting chimeric antibody is reduced as compared with the parental (for example, mouse-derived) antibody.

In the present application, the term “humanized antibody” generally refers to an antibody in which some or all of the amino acids outside the CDR of a non-human antibody (such as a mouse antibody) have been replaced by corresponding amino acids derived from human immunoglobulins. In the CDR, small additions, deletions, insertions, substitutions, or modifications to the amino acids may also be allowed, as long as they still retain the capability of the antibody to bind to a specific antigen. The humanized antibody may optionally comprise at least a portion of a constant region of a human immunoglobulin. A “humanized antibody” may retain the antigen specificity similar to that of the original antibody. The “humanized” form of a non-human (for example, mouse) antibody may minimally comprise a chimeric antibody derived from a non-human immunoglobulin sequence. In some cases, CDR residues in a human immunoglobulin (receptor antibody) may be replaced with CDR residues from a non-human species (donor antibody) (such as a mouse, a rat, a rabbit, or a non-human primate) with the desired properties, affinity, and/or capability. In some cases, FR residues of the human immunoglobulin may be replaced with corresponding non-human residues. In addition, the humanized antibody may comprise amino acid modifications that are not present in a receptor antibody or in a donor antibody. These modifications may be made to further improve the properties such as binding affinity of the antibody.

In the present application, the term “fully human antibody” generally refers to an antibody that comprises human immunoglobulin sequences only. A fully human antibody may comprise murine glycans if it is produced in mice, in mouse cells, or in hybridomas derived from mouse cells. Similarly, “murine antibody,” “mouse antibody,” or “rat antibody” refers to an antibody that comprises mouse or rat immunoglobulin sequences only, respectively. The fully human antibody may be produced in humans and in transgenic animals with human immunoglobulin germline sequences by phage display or other molecular biological methods. Exemplary techniques that can be used to manufacture antibodies are known in the art.

In the present application, the term “antigen-binding protein” generally refers to a protein comprising a moiety that binds to an antigen, and optionally a scaffold or skeleton moiety that allows the moiety that binds to the antigen to adopt a conformation that promotes the binding of the antigen-binding protein to the antigen. Examples of the antigen-binding protein comprise, but are not limited to, antibodies, antigen-binding fragments (Fab, Fab′, F(ab)2, Fv fragments, F(ab′)2, scFv, di-scFv, VHH and/or dAb), immune conjugates, multispecific antibodies (e.g., bispecific antibodies), antibody fragments, antibody derivatives, antibody analogs or fusion proteins, etc., as long as they demonstrate the desired antigen-binding activity. The “antigen-binding protein” of the present application may comprise an antigen-binding fraction and optionally, a scaffold or construct fraction allowing the antigen-binding fraction to adopt a conformation that facilitates the binding of the antigen-binding fraction to an antigen.

The term “about” generally refers to a numerical range that is 20% more or less than a specified value. For example, “about X” comprises a numeric range of X±20%, ±10%, ±5%, ±2%, ±1%, ±0.5%, ±0.2% or ±0.1%, with X being a numerical value.

### DETAILED DESCRIPTION OF THE INVENTION

The prerequisite for early detection and early treatment of presenile dementia is to find its specific diagnostic markers. In the study, the inventor has found that the DIR protein is present in the blood of presenile dementia patients, but is not detected in the blood samples of normal humans. This result shows that DIR is likely to become a blood marker for the diagnosis (possibly early diagnosis) of presenile dementia. At the same time, the inventor also tested the blood samples from other patients with cognitive impairment and found the DIR protein level increased significantly in the blood samples from patients with MCI (mild cognitive impairment) and other cognitive impairments. Therefore, increased DIR in blood will be a new marker in the diagnosis and treatment of presenile dementia.

Specifically, under pathological conditions (with such as tumors, neurodegenerative diseases, etc.), the diseased tissue experiences hypoxia due to lack of blood supply, leading to a series of changes in gene expression, and at the same time, abnormal splicing forms appear. How to find the specific genes and their splicing forms may be the key to the treatment of diseases. In the previous studies, the inventor has found that DIR is an abnormal protein formed by abnormal splicing in a human body and only exists in human tissues and cells. DIR is a key molecule that can adaptively regulate the functions of cells and tissues through high expression and secretion in a hypoxic cellular environment.

Therefore, the inventor hypothesized that DIR was likely to enter the blood through the blood supply system in diseased tissues. Subsequently, the inventor further proved the correctness of this hypothesis through the following experiments: the inventor first transferred DIR plasmids into HEK293 cells, and then detected the presence of a large amount of DIR proteins in the medium, proving that DIR can be secreted into the cells; and afterwards, DIR was subsequently detected in the plasma of AD and MCI patients, while no DIR or very low levels of DIR were detected in the plasma from normal humans and non-AD patients. This result shows that DIR specifically exists in the blood of AD and MCI patients and can be used as a blood marker for AD diagnosis (e.g., early diagnosis); and at the same time, DIR was found increased significantly in the detected blood samples from other patients with cognitive impairment. Therefore, the DIR level in the blood can be used as a new marker in the diagnosis and treatment of AD (presenile dementia).

All numerical ranges provided throughout the present application are intended to clearly comprise all numbers falling between the endpoints of the range and the numerical ranges falling therebetween. Features mentioned in the present application or in the examples can be combined. All features disclosed in the present application can be used in any combination form, and each feature disclosed in the specification can be replaced by any alternative feature capable of providing the same, equivalent or similar purpose. Therefore, unless otherwise stated, the features disclosed are only general examples of equivalent or similar features.

As used in the present application, “comprise”, “have” or “include” comprises “comprise”, “consists essentially of”, “consists substantially of” and “consist of”; and “consists essentially of”, “consists substantially of” and “consist of” are subordinate concepts of “comprise”, “have” or “include”.

In the present application, the neurodegenerative disease may comprise an acute neurodegenerative disease and a chronic neurodegenerative disease. For example, the neurodegenerative disease comprises a neurodegenerative disease caused by neuronal death and glial cell homeostasis, a neurodegenerative disease caused by aging, a neurodegenerative disease caused by affected CNS cell function, a neurodegenerative disease caused by abnormal intercellular communication and/or a neurodegenerative disease caused by impaired cell mobility.

In the present application, the neurodegenerative disease may comprise presenile dementia. For example, the neurodegenerative disease may comprise early presenile dementia, intermediate presenile dementia and/or late presenile dementia.

In the present application, the cognitive impairment may comprise mild cognitive impairment (MCI), intermediate cognitive impairment and late cognitive impairment. For example, the cognitive impairment may comprise cognitive impairment caused by normal aging, Lewy body dementia (LBD), frontotemporal dementia and/or vascular dementia. For example, the cognitive impairment-inducing diseases may induce diseases comprise Alzheimer's disease, multi-infarct type, Parkinson's disease, AIDS and/or Creutzfeldt-Jakob disease (CJD).

In the present application, the cognitive impairment may comprise mild cognitive impairment (MCI) (e.g., loss of short-term memory, difficulty in expressing or understanding abstract things, erratic mood or behavior, difficulty in learning new things and following complex instructions, impaired judgment and/or needing reminders from others for basic self-care), intermediate cognitive impairment (e.g., confusion between long-term memory and memory of real-life situations, poor expression, changes in behavior or personality, or emotional instability and/or need for assistance in basic self-care) or late cognitive impairment (e.g., memory impairment, decline in physical activity and mental status, inability to express or communicate effectively, inability to take care of oneself, and/or circadian disorder).

In the present application, the cognitive impairment may comprise suffering from a disease that may cause the induction of the cognitive impairment. For example, the subject may suffer from Alzheimer's disease, multi-infarct type, Parkinson's disease, AIDS and/or Creutzfeldt-Jakob disease (CJD).

### Diagnostic Target DIR

In one aspect, the present application provides use of a DNA damage-inducible transcript 4-like (DDIT4L) intron retention spliced product (DIR), and/or a nucleic acid encoding the DIR, in diagnosis of a disease and/or in evaluation of progression of the disease, wherein the disease may comprise cognitive impairment.

In one aspect, the present application provides use of a DNA damage-inducible transcript 4-like (DDIT4L) intron retention spliced product (DIR), and/or a nucleic acid encoding the DIR, in diagnosis of a disease and/or in evaluation of progression of the disease, wherein the disease may comprise a neurodegenerative disease.

DDIT4L (DNA Damage Inducible Transcript 4 Like, SEQ ID NO: 92) is a protein-encoding gene related to an mTOR signal transduction pathway, and its levels in macrophages may be up-regulated by means of oxidized LDL and hypoxia.

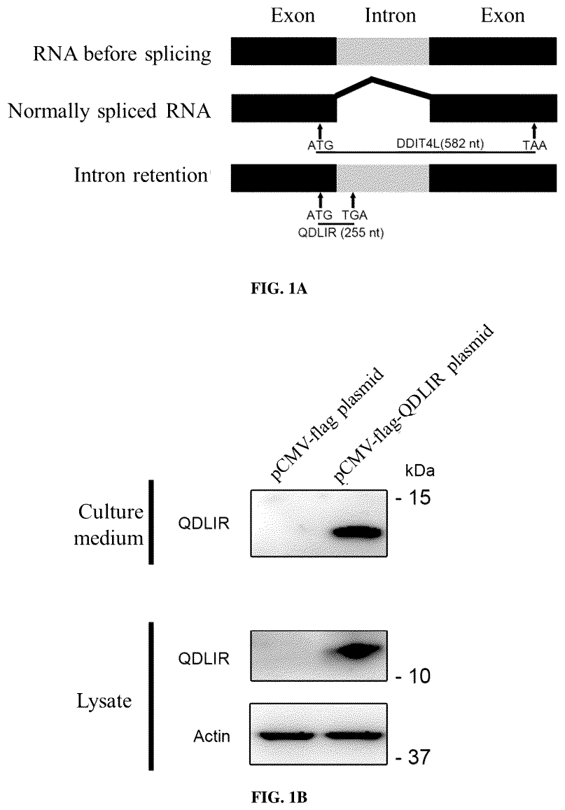

After removing an intron from the DDIT4L gene through normal splicing, a nucleotide sequence (SEQ ID NO: 94) encoding a 193aa long DDIT4L protein (SEQ ID NO: 95) may be obtained. However, the applicant has unexpectedly found that there is another special splicing mode for this gene, i.e., intron retention splicing. With this special splicing mode, 2212 nt of nucleotide sequence is obtained, and an 84aa spliced product peptide (SEQ ID NO: 1) is finally obtained since the intron may comprise a terminator. In the present application, the protein obtained by intron retention splicing may be referred to as “QDLIR or DIR” (as shown in FIG. 1A).

As used in the present application, unless otherwise stated or expressly identifiable, the term “DIR” may comprise a mature mRNA, spliced peptide encoding molecules or spliced product peptides (or DIR proteins) obtained by intronic retention splicing of the DDIT4L gene.

When the spliced product can be mRNA, it can be selected from: (a) a nucleotide molecule with a sequence as set forth in SEQ ID NO: 93; or (b) a nucleotide molecule with a high sequence identity (e.g., which may be at least 80%, 85%, 90%, 92%, 95%, 98%, 99%, 99.5%, or 99.8%) to the nucleotide molecule of SEQ ID NO: 93.

When the spliced product can be a DIR encoding molecule, it can be selected from: (a) a nucleotide molecule with a sequence as set forth in SEQ ID NO: 8; or (b) a nucleotide molecule with a high sequence identity (e.g., which may be at least 80%, 85%, 90%, 92%, 95%, 98%, 99%, 99.5%, or 99.8%) to the nucleotide molecule of SEQ ID NO: 8.

When the spliced product can be a DIR protein, it can be selected from: (a′) a polypeptide molecule with a sequence as set forth in SEQ ID NO: 1; or (b′) a protein molecule with a high sequence identity (e.g., which may be at least 80%, 85%, 90%, 92%, 95%, 98%, 99%, 99.5%, or 99.8%) to the polypeptide molecule of SEQ ID NO: 1; (c′) a protein molecule translated from any nucleotide molecule in (a) or (b) above; or (d′) a conserved variant polypeptide of the protein molecule described in any one of (a′) to (c′) above, or a homologous protein or polypeptide thereof, or an active fragment thereof.

### Diagnostic Substance and Diagnostic Kit

In another aspect, the present application provides use of a substance for detecting a DNA damage-inducible transcript 4-like DDIT4L intron retention spliced product DIR, and/or a nucleic acid encoding said DIR, in preparation of a product for diagnosis of a disease and/or in evaluation of progression of said disease, wherein said disease may comprise cognitive impairment.

In another aspect, the present application provides use of a substance for detecting a DNA damage-inducible transcript 4-like (DDIT4L) intron retention spliced product (DIR), and/or a nucleic acid encoding the DIR, in preparation of a product for diagnosis of a disease and/or in evaluation of progression of the disease, wherein the disease may comprise a neurodegenerative disease.

As used in the present application, the terms “detection substance”, “detection reagent” or “reagent for detecting DIR” or “reagent for detecting DIR expression level” are used interchangeably, and they all refer to substances that specifically target DIR molecules and can be used to directly or indirectly detect the presence and/or content of DIR molecules. These detection substances can detect DIR at the gene level or protein level.

In the present application, the substance may comprise an antigen-binding protein capable of specifically binding to the DIR, a probe capable of specifically binding to the DIR, a primer capable of specifically amplifying the nucleic acid encoding the DIR, a gene chip capable of specifically detecting the nucleic acid encoding the DIR, an aptamer capable of specifically binding to the nucleic acid encoding the DIR, and/or a guide RNA capable of specifically binding to the nucleic acid encoding the DIR.

In the present application, the kit may comprise at least one (e.g., 1, 2, 3 or more) detection substance. For example, the kit may comprise at least one (e.g., 1, 2, 3, or more) antigen-binding protein (e.g., a DIR antibody) capable of specifically binding to DIR. In the present application, different antigen-binding proteins existing in the amino acid sequence of at least one CDR may be considered as different species of antigen-binding proteins.

Depending on the sequence of the DIR molecule, those of ordinary skill in the art can prepare a reagent specifically targeting the DIR molecule based on conventional means or obtain the reagent commercially. For example, the detection reagent that can be used in the present application may include, but are not limited to: antigen-binding proteins (for example, monoclonal antibodies), probes, gene chips, PCR primers, gRNA or others having detection specificity for DIR molecules.

Moreover, for the sake of detection, the detection reagent of the present application may also come with detectable labels, which may include, but are not limited to: radioactive isotopes, fluorophores, chemiluminescent moieties, enzymes, enzyme substrates, enzyme cofactors, enzyme inhibitors, dyes, metal ions, ligands (such as biotins or haptens), etc.

The detection reagent of the present application may exist in a solution, be fixed on a carrier (such as a substrate or an adsorbent), or exist in other conventional modes in the art, as long as its mode of existence is suitable for DIR detection in biological samples. For example, when the detection reagent of the present application can be a nucleotide probe, it may exist in the form of a biochip (or “microarray”).

In the present application, the product may exist in a kit form.

The present application further provides a product for diagnosis of presenile dementia and/or evaluation of progression (e.g., a grade or stage) of the presenile dementia. The product may comprise: a substance for detecting a DIR level, and optionally, other substances for diagnosis of presenile dementia and/or evaluation of progression (e.g., a grade or stage) of presenile dementia, for example, detection substances for existing AD markers.

In the present application, the diagnostic kit may comprise an instruction manual recording a method of using the diagnostic kit for diagnosis of cognitive impairment, and/or evaluation of progression of the cognitive impairment; and/or for diagnosis of a neurodegenerative disease, and/or evaluation of progression of the neurodegenerative disease.

According to the needs of the detection method used, an appropriate DIR detection substance may be selected and prepared into a product (such as a kit) suitable for the detection method used. Those of ordinary skill in the art can adjust and change the detection method and the reagent contained in the product according to actual conditions and needs.

Accordingly, the present application further provides a product (such as a detection kit), which may comprise: (i) a detection effective amount of one or more reagents for detecting DIR; and (ii) optionally, one or more substances selected from the group consisting of: a container, an instruction manual, a positive control, a negative control, a buffer, an auxiliary or solvent, for example, a solution for suspending or fixing cells, a detectable label or marker, a solution facilitating hybridization of nucleic acids, a solution for cell lysis, or a solution for nucleic acid purification.

In one example, the present application provides a detection kit suitable for detecting the expression of a DIR protein in a biological sample by immunohistochemistry. The detection kit may comprise: a blocking solution, for example, 10% goat serum; a primary antibody, for example, a rabbit anti-human or mouse DIR monoclonal antibody; a secondary antibody, for example, a labeled (such as HRP labeled) or unlabeled goat anti-rabbit secondary antibody; a substrate buffer, for example, a DAB substrate buffer; a developing solution; and optionally, a container containing the above reagents and an instruction manual.

The diagnostic kit of the present application may also come with an instruction manual of the kit, which records how to detect with the kit, and how to diagnose cognitive impairment and/or evaluate the progression of the cognitive impairment, and/or diagnose a neurodegenerative disease and/or evaluate the progression of the neurodegenerative disease, by using a detection result. For example, the kit described in the present application can be used for diagnosis of AD and/or evaluation of progression (e.g., a grade or stage) of AD.

The product of the present application may further comprise other reagents that are clinically used for diagnosis of AD and/or evaluation of AD progression (e.g., grading or staging) in a subject, in order to assist or verify the results obtained by detecting DIR. Those of ordinary skill in the art can make routine selections based on specific needs.

### Diagnosis Method

In another aspect, the present application provides a method for diagnosing cognitive impairment, and/or evaluating progression of the cognitive impairment. The method may comprise the step of: detecting, from a sample of a subject in need thereof, a content of a DNA damage-inducible transcript 4-like (DDIT4L) intron retention spliced product DIR, and/or of a nucleic acid encoding the DIR.

In another aspect, the present application provides a method for diagnosing a neurodegenerative disease, and/or evaluating progression of the neurodegenerative disease. The method may comprise the step of: detecting, from a sample of a subject in need thereof, a content of a DNA damage-inducible transcript 4-like (DDIT4L) intron retention spliced product DIR, and/or of a nucleic acid encoding the DIR.

In the present application, the subject may comprise a mammal.

In the present application, the subject may comprise a tumor patient.

In the present application, the subject is in an old age stage.

In the present application, the sample may comprise a blood sample and/or a tissue or cell sample.

In the present application, the sample may comprise whole blood, serum, plasma, and/or cerebrospinal fluid.

In the present application, the method may comprise: comparing the content of the DIR and/or of the nucleic acid encoding the DIR in the sample of the subject with a normal control value, wherein the normal control value may comprise a content of the DIR and/or of the nucleic acid encoding the DIR in a normal subject.

In the present application, the method may be used to diagnose cognitive impairment and/or evaluate the progression of the cognitive impairment by using the kit of the present application, and/or the detection substance of the present application.

In the present application, the normal subject may not suffer from the cognitive impairment. The normal subject may not suffer from the neurodegenerative disease.

In the present application, when the content of the DIR and/or of the nucleic acid encoding the DIR in the sample of the subject is significantly higher (for example, it can be increased by at least about 20%, at least about 50%, at least about 60%, at least about 65%, at least about 70%, at least about 75%, at least about 80%, at least about 85%, at least about 90%, at least about 95%, at least about 100%, at least about 200%, at least about 500% or more higher) than the normal control value, the subject may be diagnosed with the cognitive impairment and/or the neurodegenerative disease.

In the present application, the method may comprise: comparing the content of the DIR and/or of the nucleic acid encoding the DIR in the sample of the subject with an early control value, wherein the early control value may comprise a previously measured content of the DIR and/or of the nucleic acid encoding the DIR in the same subject.

In the present application, the subject has been diagnosed with the cognitive impairment and/or the neurodegenerative disease. For example, the subject has been diagnosed with the cognitive impairment; or the subject has been diagnosed with the neurodegenerative disease.

In the present application, when the content of the DIR and/or of the nucleic acid encoding the DIR in the sample of the subject is significantly higher (for example, it can be increased by at least about 20%, at least about 50%, at least about 60%, at least about 65%, at least about 70%, at least about 75%, at least about 80%, at least about 85%, at least about 90%, at least about 95%, at least about 100%, at least about 200%, at least about 500% or more higher) than the early control value, the subject may be diagnosed with aggravated progression of the cognitive impairment and/or the neurodegenerative disease.

In the present application, the method may further comprise the step of detecting a content of a marker associated with the cognitive impairment and/or the neurodegenerative disease in a sample derived from the subject.

In the present application, the marker associated with the cognitive impairment and/or the neurodegenerative disease may further comprise AD7C-NTP, pTau-181, pTau-217, Aβ40 and/or Aβ42. The detection result of the content of the marker may be used to assist the diagnosis of cognitive impairment, and/or the evaluation of progression of the cognitive impairment, and/or for use in diagnosis of a neurodegenerative disease, and/or the evaluation of progression of the neurodegenerative disease.

In the present application, the method may further comprise the step of observing brain imaging of the subject. The result of the brain imaging may be used to assist the diagnosis of cognitive impairment, and/or the evaluation of progression of the cognitive impairment, and/or for use in diagnosis of a neurodegenerative disease, and/or the evaluation of progression of the neurodegenerative disease.

### Diagnosis System

In another aspect, the present application provides a system for diagnosing cognitive impairment, and/or evaluating progression of the cognitive impairment. The system may comprise a substance for detecting a DNA damage-inducible transcript 4-like (DDIT4L) intron retention spliced product (DIR), and/or a nucleic acid encoding the DIR.

In another aspect, the present application provides a system for diagnosing a neurodegenerative disease, and/or evaluating progression of the neurodegenerative disease. The system may comprise a substance for detecting a DNA damage-inducible transcript 4-like (DDIT4L) intron retention spliced product (DIR), and/or a nucleic acid encoding the DIR.

In the present application, the system may comprise a detection module capable of detecting a content value of the DIR and/or of the nucleic acid encoding the DIR.

In the present application, the detection module detects a content value of the DIR and/or of the nucleic acid encoding the DIR in the sample acquired by the collection module.

In the present application, the detection module may comprise a substance capable of detecting a content value of the DIR and/or of the nucleic acid encoding the DIR.

In the present application, the substance may comprise an antigen-binding protein capable of specifically binding to the DIR, a probe capable of specifically binding to the DIR, a primer capable of specifically amplifying the nucleic acid encoding the DIR, a gene chip capable of specifically detecting the nucleic acid encoding the DIR, an aptamer capable of specifically binding to the nucleic acid encoding the DIR, and/or a guide RNA capable of specifically binding to the nucleic acid encoding the DIR.

In the present application, the detection module may comprise at least one (e.g., 1, 2, 3 or more) detection substance. For example, the detection module may comprise at least one (e.g., 1, 2, 3, or more) antigen-binding protein (e.g., a DIR antibody) capable of specifically binding to DIR. In the present application, different antigen-binding proteins existing in the amino acid sequence of at least one CDR may be considered as different species of antigen-binding proteins.

In the present application, the detection module may comprise an instrument capable of detecting a content of the DNA damage-inducible transcript 4-like (DDIT4L) intron retention spliced product (DIR), and/or of the nucleic acid encoding the DIR. For example, the instrument may comprise an instrument required to perform qPCR, ELISA and western blotting, and/or a gene chip.

In the present application, the detection module may comprise a substance and/or an instrument capable of detecting a content of a marker associated with the cognitive impairment and/or the neurodegenerative disease.

In the present application, the marker associated with the cognitive impairment and/or the neurodegenerative disease may further comprise AD7C-NTP, pTau-181, pTau-217, Aβ40 and/or Aβ42. For example, the substance may further comprise an antibody capable of specifically binding to the marker, a probe capable of specifically binding to the marker, a primer capable of specifically amplifying the nucleic acid encoding the marker, a gene chip capable of specifically detecting the nucleic acid encoding the marker, an aptamer capable of specifically binding to the nucleic acid encoding the marker, and/or a guide RNA capable of specifically binding to the nucleic acid encoding the marker. For example, the instrument may comprise an instrument required to perform qPCR, ELISA and western blotting, and/or a gene chip.

In the present application, the detection module may output a content value of the DIR and/or of the nucleic acid encoding the DIR.

In the present application, the system may comprise a determination module capable of determining a diagnosis result and/or a disease progression result of said subject based on the content of said DIR and/or of said nucleic acid encoding said DIR as well as said normal control value and/or said early control value; the normal control value may comprise a content of the DIR and/or of the nucleic acid encoding the DIR in a normal subject; and wherein the early control value may comprise a previously measured content of the DIR and/or of the nucleic acid encoding the DIR in the same subject.

In the present application, the “previously” may include at least 10 days ago, at least 20 days ago, at least 1 month ago, at least 6 months ago, at least 12 months ago, at least 24 months ago or more ago. In the present application, the subject may have been diagnosed with the cognitive impairment and/or the neurodegenerative disease. With the system of the present application, the content of the DIR and/or of the nucleic acid encoding the DIR in the subject at different time stages may be traced, thereby helping to diagnose the progression of the cognitive impairment and/or of the neurodegenerative disease of the subject.

In the present application, the normal subject may not suffer from the cognitive impairment and/or the neurodegenerative disease.

For example, when the content of the DIR and/or of the nucleic acid encoding the DIR in the sample of the subject is significantly higher (for example, it can be increased by at least about 20%, at least about 50%, at least about 60%, at least about 65%, at least about 70%, at least about 75%, at least about 80%, at least about 85%, at least about 90%, at least about 95%, at least about 100%, at least about 200%, at least about 500% or more higher) than the normal control value, the determination module may diagnose that the subject may suffer from the cognitive impairment and/or the neurodegenerative disease.

For example, when the content of the DIR and/or of the nucleic acid encoding the DIR in the sample of the subject is significantly higher (for example, it can be increased by at least about 20%, at least about 50%, at least about 60%, at least about 65%, at least about 70%, at least about 75%, at least about 80%, at least about 85%, at least about 90%, at least about 95%, at least about 100%, at least about 200%, at least about 500% or more higher) than the early control value, the determination module may diagnose that the subject may have aggravated progression of the cognitive impairment and/or the neurodegenerative disease.

In the present application, the determination module may output a diagnosis result of the subject.

In the present application, the system may comprise a display module capable of displaying the diagnosis result of the determination module. For example, the diagnostic results displayed by the display module may comprise qualitative and/or quantitative results.

In the present application, the system may comprise a storage module capable of storing the content value output by the detection module, and/or the diagnosis result output by the determination module.

In the present application, the system may comprise the following devices:
a) a device for collecting and/or receiving data on a DIR level in a sample; and
b) a device for analyzing the data for diagnosis of AD and/or for evaluation of progression of AD in the subject, wherein DIR being higher than a control level indicates the subject suffers from AD or undergoes some development of AD.

In the present application, the system may further comprise (a′) a device for collecting and/or receiving AD diagnosis and/or data on other clinical indicators or basic characteristics of the development of the AD in a sample.

In the present application, the device described in (b) is used to combine and analyze DIR level data with data on other clinical indicators or basic characteristics of AD diagnosis and/or development progression of AD, in order to perform prognosis evaluation on the AD diagnosis and/or AD development progression of the subject.

Or, in the present application, the system may further comprise a device for analyzing AD diagnosis and/or data on other clinical indicators or basic characteristics of the progression of AD; and/or a device for combining and analyzing DIR level data with data on other clinical indicators or basic characteristics of AD diagnosis and/or development progression of AD, in order to evaluate the AD diagnosis and/or the progression of AD of the subject.

In the present application, the system may further comprise one or more devices selected from the group consisting of: a device for inputting and/or outputting and/or storing data; a device for outputting and/or storing analysis results, for example, a device for uploading and/or storing the analysis results to a cloud database or its corresponding cloud database device; and a device for performing intelligent analysis and recommending a therapeutic scheme based on evaluation results.

It should be understood that those skilled in the art may add other conventional devices or programs to the system as needed.

Generally, the diagnosis of AD and/or the evaluation of progression (e.g., grading or staging) of AD may be performed with a method including: detecting the level of DIR molecules in a subject or a sample derived from the subject, and comparing the level with a control level; and if a comparison result shows that the level of DIR molecules in the subject is higher than the control level, providing a prompt that the subject suffers from AD or has some development of AD. In some embodiments, the method of the present application optionally further comprises: obtaining a sample to be tested from the subject; and contacting the sample to be tested with a reagent or kit for detecting the DIR level.

As used herein, the term “control” may comprise a normal control or a control with a specific developmental grade or stage of AD. “Control” may refer to the level of a DIR molecule used as a reference, including but not limited to, a DIR molecule level measured in a non-AD normal biological sample from the same subject (e.g., a sample derived from the subject who is not an AD patient or is a normal human), a statistically determined population standard level, or a standardized level. The term “control with a specific developmental grade or stage of AD” refers to a DIR molecule level measured in the same subject at a previously determined developmental grade or stage, a statistically determined population standard level at that grade or stage, or a normalized s level.

### Antigen-Binding Protein of DIR

In the present application, the antigen-binding protein may comprise LCDR2, which may comprise the amino acid sequence as set forth in SEQ ID NO: 74. For example, the antigen-binding protein of the present application may have DIR binding capability. For example, the CDR may be determined by the IMGT numbering scheme.

X₁ X₂S (SEQ ID NO.74), with X₁ being A or Y and X₂ being A or Y.

In the present application, the LCDR2 may comprise the amino acid sequence as set forth in SEQ ID NO: 26 or 16. The amino acid sequence of SEQ ID NO: 16 is YTS, and the amino acid sequence of SEQ ID NO: 26 is AAS.

In the present application, the antigen-binding protein may comprise LCDR1, which may comprise the amino acid sequence as set forth in SEQ ID NO: 73. For example, the antigen-binding protein of the present application may have DIR binding capability. For example, the CDR may be determined by the IMGT numbering scheme. For example, the LCDR1 may comprise LCDR1s in the light chain variable regions of IR-II-1 to IR-II-10.

Q X₁ X₂D X₃ X₄ X₅ X₆ X₇Y (SEQ ID NO.73), with X₁ being absent or S, X₂ being absent or V, X₃ being absent or Y, X₄ being absent or D, X₅ being G or I, X₆ being D, E or S, and X₇ being N or S.

In the present application, the LCDR1 may comprise the amino acid sequence as set forth in any one of SEQ ID NOs: 25, 52 and 15.

In the present application, the antigen-binding protein may comprise LCDR3, which may comprise the amino acid sequence as set forth in SEQ ID NO: 75. For example, the antigen-binding protein of the present application may have DIR binding capability. For example, the CDR may be determined by the IMGT numbering scheme. For example, the LCDR3 may comprise LCDR3s in the light chain variable regions of IR-II-1 to IR-II-10.

X₁Q X₂ X₃ X₄ X₅P X₆ X₇(SEQ ID NO.75), with X₁ being L or Q, X₂ being S or Y, X₃ being N, S or Y, X₄ being D, E or K, X₅ being D or L, X₆ being F or R, and X₇ being A or T.

In the present application, the LCDR3 may comprise the amino acid sequence as set forth in any one of SEQ ID NOs: 27, 53, 67 and 17.

In the present application, the antigen-binding protein may comprise LCDR3, LCDR2 and LCDR1. For example, in the antigen-binding protein, the LCDR2 may comprise the amino acid sequence as set forth in SEQ ID NO: 74, the LCDR1 may comprise the amino acid sequence as set forth in SEQ ID NO: 73, and the LCDR3 may comprise the amino acid sequence as set forth in SEQ ID NO: 75. For example, the antigen-binding protein of the present application may have DIR binding capability. For example, the CDR may be determined by the IMGT numbering scheme. For example, the LCDR1 to LCDR3 may comprise LCDR1s to LCDR3s in the light chain variable regions of IR-II-1 to IR-II-10.

In the present application, the antigen-binding protein may comprise LCDR3, LCDR2 and LCDR1. For example, in the antigen-binding protein, the LCDR1 may comprise the amino acid sequence as set forth in SEQ ID NO: 25, the LCDR2 may contain the amino acid sequence as set forth in SEQ ID NO: 26, and the LCDR3 may comprise the amino acid sequence as set forth in SEQ ID NO: 27.

For example, in the antigen-binding protein, the LCDR1 may comprise the amino acid sequence as set forth in SEQ ID NO: 52, the LCDR2 may contain the amino acid sequence as set forth in SEQ ID NO: 26, and the LCDR3 may comprise the amino acid sequence as set forth in SEQ ID NO: 53.

For example, in the antigen-binding protein, the LCDR1 may comprise the amino acid sequence as set forth in SEQ ID NO: 52, the LCDR2 may contain the amino acid sequence as set forth in SEQ ID NO: 26, and the LCDR3 may comprise the amino acid sequence as set forth in SEQ ID NO: 67.

For example, in the antigen-binding protein, the LCDR1 may comprise the amino acid sequence as set forth in SEQ ID NO: 15, the LCDR2 may comprise the amino acid sequence as set forth in SEQ ID NO: 16, and the LCDR3 may comprise the amino acid sequence as set forth in SEQ ID NO: 17.

For example, the antigen-binding protein of the present application may have DIR binding capability. For example, the CDR may be determined by the IMGT numbering scheme. For example, the LCDR1 to LCDR3 may comprise LCDR1s to LCDR3s in the heavy chain variable regions of IR-II-1 to IR-II-10.

In the present application, the antigen-binding protein may comprise HCDR1, which may comprise the amino acid sequence as set forth in SEQ ID NO: 70. For example, the antigen-binding protein of the present application may have DIR binding capability. For example, the CDR may be determined by the IMGT numbering scheme. For example, the HCDR1 may comprise HCDR1s in the heavy chain variable regions of IR-II-1 to IR-II-10.

GYTFX₁X₂YX₃ (SEQ ID NO.70), with X₁ being S or T, X₂ being E, N, R or S, X₃ being T or W.

In the present application, the HCDR1 may comprise the amino acid sequence as set forth in any one of SEQ ID NOs: 20, 35, 56 and 10.

In the present application, the antigen-binding protein may comprise HCDR2, which may comprise the amino acid sequence as set forth in SEQ ID NO: 71. For example, the antigen-binding protein of the present application may have DIR binding capability. For example, the CDR may be determined by the IMGT numbering scheme. For example, the HCDR2 may comprise HCDR2s in the heavy chain variable regions of IR-II-1 to IR-II-10.

I X₁P X₂ X₃ X₄ X₅ X₆T (SEQ ID NO.71), with X₁ being L or Y, X₂ being G or H, X₃ being G, N or S, X₄ being absent or Y, X₅ being G or Y, X₆ being G, N, S or T.

In the present application, the HCDR2 may comprise the amino acid sequence as set forth in any one of SEQ ID NOs: 21, 36, 42, 48 and 11.

In the present application, the antigen-binding protein may comprise HCDR3, which may comprise the amino acid sequence as set forth in SEQ ID NO: 72. For example, the antigen-binding protein of the present application may have DIR binding capability. For example, the CDR may be determined by the IMGT numbering scheme. For example, the HCDR3 may comprise HCDR3s in the heavy chain variable regions of IR-II-1 to IR-II-10.

X₁ X₂ X₃ X₄ X₅ X₆ X₇ X₈ X₉ X₁₀ X₁₁ X₁₂ X₁₃ X₁₄ X₁₅DY (SEQ ID NO.72), with X₁ being absent or A, X₂ being absent or R, X₃ being absent, S or T, X₄ being absent, D, G or Y, X₅ being absent, D, I, M or V, X₆ being absent, G or I, X₇ being absent, T or Y, X₈ being absent, A, L or T, X₉ being absent, R, S or T, X₁₀ being absent, G or T, X₁₁ being absent, D or E, X₁₂ being D or Y, X₁₃ being F, L or Y, X₁₄ being A, T or V, X₁₅ being F or M.

In the present application, the HCDR3 may comprise the amino acid sequence as set forth in any one of SEQ ID Nos: 22, 30, 37, 43, 49, 57, 62, and 12.

In the present application, the antigen-binding protein may comprise HCDR3, HCDR2 and HCDR1. For example, in the antigen-binding protein, the HCDR2 may comprise the amino acid sequence as set forth in SEQ ID NO: 71, the HCDR1 may comprise the amino acid sequence as set forth in SEQ ID NO: 70, and the HCDR3 may comprise the amino acid sequence as set forth in SEQ ID NO: 72. For example, the antigen-binding protein of the present application may have DIR binding capability. For example, the CDR may be determined by the IMGT numbering scheme. For example, the HCDR1 to HCDR3 may comprise HCDR1s to HCDR3s in the heavy chain variable regions of IR-II-1 to IR-II-10.

For example, in the antigen-binding protein, the HCDR1 may comprise the amino acid sequence as set forth in SEQ ID NO: 20, the HCDR2 may comprise the amino acid sequence as set forth in SEQ ID NO: 21, and the HCDR3 may comprise the amino acid sequence as set forth in SEQ ID NO: 22.

For example, in the antigen-binding protein, the HCDR1 may comprise the amino acid sequence as set forth in SEQ ID NO: 20, the HCDR2 may comprise the amino acid sequence as set forth in SEQ ID NO: 21, and the HCDR3 may comprise the amino acid sequence as set forth in SEQ ID NO: 30.

For example, in the antigen-binding protein, the HCDR1 may comprise the amino acid sequence as set forth in SEQ ID NO: 35, the HCDR2 may comprise the amino acid sequence as set forth in SEQ ID NO: 36, and the HCDR3 may comprise the amino acid sequence as set forth in SEQ ID NO: 37.

For example, in the antigen-binding protein, the HCDR1 may comprise the amino acid sequence as set forth in SEQ ID NO: 20, the HCDR2 may comprise the amino acid sequence as set forth in SEQ ID NO: 42, and the HCDR3 may comprise the amino acid sequence as set forth in SEQ ID NO: 43.

For example, in the antigen-binding protein, the HCDR1 may comprise the amino acid sequence as set forth in SEQ ID NO: 20, the HCDR2 may comprise the amino acid sequence as set forth in SEQ ID NO: 48, and the HCDR3 may comprise the amino acid sequence as set forth in SEQ ID NO: 49.

For example, in the antigen-binding protein, the HCDR1 may comprise the amino acid sequence as set forth in SEQ ID NO: 56, the HCDR2 may comprise the amino acid sequence as set forth in SEQ ID NO: 36, and the HCDR3 may comprise the amino acid sequence as set forth in SEQ ID NO: 57.

For example, in the antigen-binding protein, the HCDR1 may comprise the amino acid sequence as set forth in SEQ ID NO: 35, the HCDR2 may comprise the amino acid sequence as set forth in SEQ ID NO: 36, and the HCDR3 may comprise the amino acid sequence as set forth in SEQ ID NO: 62.

For example, in the antigen-binding protein, the HCDR1 may comprise the amino acid sequence as set forth in SEQ ID NO: 35, the HCDR2 may comprise the amino acid sequence as set forth in SEQ ID NO: 42, and the HCDR3 may comprise the amino acid sequence as set forth in SEQ ID NO: 37.

For example, in the antigen-binding protein, the HCDR1 may comprise the amino acid sequence as set forth in SEQ ID NO: 56, the HCDR2 may comprise the amino acid sequence as set forth in SEQ ID NO: 36, and the HCDR3 may comprise the amino acid sequence as set forth in SEQ ID NO: 57.

For example, in the antigen-binding protein, the HCDR1 may comprise the amino acid sequence as set forth in SEQ ID NO: 10, the HCDR2 may comprise the amino acid sequence as set forth in SEQ ID NO: 11, and the HCDR3 may comprise the amino acid sequence as set forth in SEQ ID NO: 12.

For example, the antigen-binding protein of the present application may have DIR binding capability. For example, the CDR may be determined by the IMGT numbering scheme. For example, the HCDR1 to HCDR3 may comprise HCDR1s to HCDR3s in the heavy chain variable regions of IR-II-1 to IR-II-10.

In the present application, the antigen-binding protein may comprise HCDR3, HCDR2, HCDR1, LCDR3, LCDR2 and LCDR1.

For example, in the antigen-binding protein of the present application, the HCDR1 may comprise the amino acid sequence as set forth in SEQ ID NO: 20, the HCDR2 may comprise the amino acid sequence as set forth in SEQ ID NO: 21, the HCDR3 may comprise the amino acid sequence as set forth in SEQ ID NO: 22, the LCDR1 may comprise the amino acid sequence as set forth in SEQ ID NO: 25, the LCDR2 may comprise the amino acid sequence as set forth in SEQ ID NO: 26, the LCDR3 may comprise the amino acid sequence as set forth in SEQ ID NO: 27.

For example, in the antigen-binding protein of the present application, the HCDR1 may comprise the amino acid sequence as set forth in SEQ ID NO: 20, the HCDR2 may comprise the amino acid sequence as set forth in SEQ ID NO: 21, the HCDR3 may comprise the amino acid sequence as set forth in SEQ ID NO: 30, the LCDR1 may comprise the amino acid sequence as set forth in SEQ ID NO: 25, the LCDR2 may comprise the amino acid sequence as set forth in SEQ ID NO: 26, the LCDR3 may comprise the amino acid sequence as set forth in SEQ ID NO: 27.

For example, in the antigen-binding protein of the present application, the HCDR1 may comprise the amino acid sequence as set forth in SEQ ID NO: 35, the HCDR2 may comprise the amino acid sequence as set forth in SEQ ID NO: 36, the HCDR3 may comprise the amino acid sequence as set forth in SEQ ID NO: 37, the LCDR1 may comprise the amino acid sequence as set forth in SEQ ID NO: 25, the LCDR2 may comprise the amino acid sequence as set forth in SEQ ID NO: 26, the LCDR3 may comprise the amino acid sequence as set forth in SEQ ID NO: 27.

For example, in the antigen-binding protein of the present application, the HCDR1 may comprise the amino acid sequence as set forth in SEQ ID NO: 20, the HCDR2 may comprise the amino acid sequence as set forth in SEQ ID NO: 42, the HCDR3 may comprise the amino acid sequence as set forth in SEQ ID NO: 43, the LCDR1 may comprise the amino acid sequence as set forth in SEQ ID NO: 25, the LCDR2 may comprise the amino acid sequence as set forth in SEQ ID NO: 26, the LCDR3 may comprise the amino acid sequence as set forth in SEQ ID NO: 27.

For example, in the antigen-binding protein of the present application, the HCDR1 may comprise the amino acid sequence as set forth in SEQ ID NO: 20, the HCDR2 may comprise the amino acid sequence as set forth in SEQ ID NO: 48, the HCDR3 may comprise the amino acid sequence as set forth in SEQ ID NO: 49, the LCDR1 may comprise the amino acid sequence as set forth in SEQ ID NO: 52, the LCDR2 may comprise the amino acid sequence as set forth in SEQ ID NO: 26, the LCDR3 may comprise the amino acid sequence as set forth in SEQ ID NO: 53.

For example, in the antigen-binding protein of the present application, the HCDR1 may comprise the amino acid sequence as set forth in SEQ ID NO: 56, the HCDR2 may comprise the amino acid sequence as set forth in SEQ ID NO: 36, the HCDR3 may comprise the amino acid sequence as set forth in SEQ ID NO: 57, the LCDR1 may comprise the amino acid sequence as set forth in SEQ ID NO: 25, the LCDR2 may comprise the amino acid sequence as set forth in SEQ ID NO: 26, the LCDR3 may comprise the amino acid sequence as set forth in SEQ ID NO: 27.

For example, in the antigen-binding protein of the present application, the HCDR1 may comprise the amino acid sequence as set forth in SEQ ID NO: 35, the HCDR2 may comprise the amino acid sequence as set forth in SEQ ID NO: 36, the HCDR3 may comprise the amino acid sequence as set forth in SEQ ID NO: 62, the LCDR1 may comprise the amino acid sequence as set forth in SEQ ID NO: 25, the LCDR2 may comprise the amino acid sequence as set forth in SEQ ID NO: 26, the LCDR3 may comprise the amino acid sequence as set forth in SEQ ID NO: 27.

For example, in the antigen-binding protein of the present application, the HCDR1 may comprise the amino acid sequence as set forth in SEQ ID NO: 35, the HCDR2 may comprise the amino acid sequence as set forth in SEQ ID NO: 42, the HCDR3 may comprise the amino acid sequence as set forth in SEQ ID NO: 37, the LCDR1 may comprise the amino acid sequence as set forth in SEQ ID NO: 52, the LCDR2 may comprise the amino acid sequence as set forth in SEQ ID NO: 26, the LCDR3 may comprise the amino acid sequence as set forth in SEQ ID NO: 53.

For example, in the antigen-binding protein of the present application, the HCDR1 may comprise the amino acid sequence as set forth in SEQ ID NO: 56, the HCDR2 may comprise the amino acid sequence as set forth in SEQ ID NO: 36, the HCDR3 may comprise the amino acid sequence as set forth in SEQ ID NO: 57, the LCDR1 may comprise the amino acid sequence as set forth in SEQ ID NO: 52, the LCDR2 may comprise the amino acid sequence as set forth in SEQ ID NO: 26, the LCDR3 may comprise the amino acid sequence as set forth in SEQ ID NO: 67.

For example, in the antigen-binding protein of the present application, the HCDR1 may comprise the amino acid sequence as set forth in SEQ ID NO: 10, the HCDR2 may comprise the amino acid sequence as set forth in SEQ ID NO: 11, the HCDR3 may comprise the amino acid sequence as set forth in SEQ ID NO: 12, the LCDR1 may comprise the amino acid sequence as set forth in SEQ ID NO: 15, the LCDR2 may contain the amino acid sequence as set forth in SEQ ID NO: 16, and the LCDR3 may comprise the amino acid sequence as set forth in SEQ ID NO: 17.

For example, the antigen-binding protein of the present application may have DIR binding capability. For example, the CDR may be determined by the IMGT numbering scheme. For example, the HCDR1 to HCDR3 may comprise HCDR1s to HCDR3s in the heavy chain variable regions of IR-II-1 to IR-II-10; and the LCDR1 to LCDR3 may comprise LCDR1s to LCDR3s in the light chain variable regions of IR-II-1 to IR-II-10.

In the present application, the antigen-binding protein may comprise H-FR1, and a C-terminus of the H-FR1 may be directly or indirectly linked to an N-terminus of the HCDR1. In the present application, the antigen-binding protein may comprise H-FR2, which may be located between the HCDR1 and the HCDR2. In the present application, the antigen-binding protein may comprise H-FR3, which may be located between the HCDR2 and the HCDR3. In the present application, the antigen-binding protein may comprise H-FR4, and an N-terminus of the H-FR4 may be linked to a C-terminus of the HCDR3. In the present application, the antigen-binding protein may comprise H-FR1, H-FR2, H-FR3 and H-FR4.

In the present application, the antigen-binding protein may comprise L-FR1, and a C-terminus of the L-FR1 may be directly or indirectly linked to an N-terminus of the LCDR1. In the present application, the antigen-binding protein may comprise L-FR1, and a C-terminus of the L-FR1 may be directly or indirectly linked to an N-terminus of the LCDR1. In the present application, the antigen-binding protein may comprise L-FR2, which may be located between the LCDR1 and the LCDR2. In the present application, the antigen-binding protein may comprise L-FR3, which may be located between the LCDR2 and the LCDR3. In the present application, the antigen-binding protein may comprise L-FR4, and an N-terminus of the L-FR4 may be linked to a C-terminus of the LCDR3. In the present application, the antigen-binding protein may comprise L-FR1, L-FR2, L-FR3 and L-FR4.

In the present application, the antigen-binding protein may comprise a heavy chain variable region VH, which may comprise the amino acid sequence as set forth in any one of SEQ ID NOs: 13, 23, 31, 38, 44, 50, 58, 63 and 65. For example, the antigen-binding protein of the present application may have DIR binding capability.

In the present application, the antigen-binding protein may comprise a light chain variable region VL, which may comprise the amino acid sequence as set forth in any one of SEQ ID NOs: 18, 28, 33, 40, 46, 54, 60 and 68. For example, the antigen-binding protein of the present application may have DIR binding capability.

In the present application, the antigen-binding protein may comprise the VH and the VL. In some embodiments, the VH may comprise the amino acid sequence as set forth in SEQ ID NO: 23, and the VL may comprise the amino acid sequence as set forth in SEQ ID NO: 28.

In some embodiments, the VH may comprise the amino acid sequence as set forth in SEQ ID NO: 31 and the VL may comprise the amino acid sequence as set forth in SEQ ID NO: 33.

In some embodiments, the VH may comprise the amino acid sequence as set forth in SEQ ID NO: 38 and the VL may comprise the amino acid sequence as set forth in SEQ ID NO: 40.

In some embodiments, the VH may comprise the amino acid sequence as set forth in SEQ ID NO: 44 and the VL may comprise the amino acid sequence as set forth in SEQ ID NO: 46.

In some embodiments, the VH may comprise the amino acid sequence as set forth in SEQ ID NO: 50 and the VL may comprise the amino acid sequence as set forth in SEQ ID NO: 54.

In some embodiments, the VH may comprise the amino acid sequence as set forth in SEQ ID NO: 63, and the VL may comprise the amino acid sequence as set forth in SEQ ID NO: 60.

In some embodiments, the VH may comprise the amino acid sequence as set forth in SEQ ID NO: 65, and the VL may comprise the amino acid sequence as set forth in SEQ ID NO: 54.

In some embodiments, the VH may comprise the amino acid sequence as set forth in SEQ ID NO: 58, and the VL may comprise the amino acid sequence as set forth in SEQ ID NO: 68.

In the present application, the VH may comprise the amino acid sequence as set forth in SEQ ID NO: 13, and the VL may comprise the amino acid sequence as set forth in SEQ ID NO: 18. For example, the antigen-binding protein of the present application may have DIR binding capability.

In the present application, the antigen-binding protein may comprise at least one CDR in the VH of the present application. In the present application, the antigen-binding protein may comprise at least one CDR in the VL of the present application. The CDR may be obtained by partitioning in any partitioning mode. In the present application, the CDR may encompass a CDR sequence obtained by partitioning in any CDR partitioning mode, as well as variations thereof.

In the present application, the antigen-binding protein may comprise HCDR1, HCDR2 and HCDR3 in the VH described in the present application.

In the present application, the antigen-binding protein may comprise HCDR1, HCDR2 and HCDR3 in the VH described in the present application. The VH may comprise the amino acid sequence as set forth in any one of SEQ ID NOs: 13, 23, 31, 38, 44, 50, 58, 63, and 65. For example, the antigen-binding protein of the present application may have DIR binding capability. In the present application, the CDR may encompass a CDR sequence obtained by partitioning in any CDR partitioning mode, as well as variations thereof.

In the present application, the antigen-binding protein may comprise LCDR1, LCDR2 and LCDR3 in the VL described in the present application. The VL may comprise the amino acid sequence as set forth in any one of SEQ ID NOs: 18, 28, 33, 40, 46, 54, 60 and 68. For example, the antigen-binding protein of the present application may have DIR binding capability. In the present application, the CDR may encompass a CDR sequence obtained by partitioning in any CDR partitioning mode, as well as variations thereof.

In the present application, the antigen-binding protein may comprise an antibody heavy chain constant region. The antibody heavy chain constant region may be derived from a human IgG, IgA, IgD, IgE and/or IgM heavy chain constant region(s). The antibody heavy chain constant region may be derived from a human IgG heavy chain constant region. In some embodiments, the antigen-binding protein may comprise an antibody heavy chain constant region, which may be derived from a human IgG1, IgG2, IgG3 and/or IgG4 heavy-chain constant region(s). In some embodiments, the antigen-binding protein may comprise an antibody heavy chain constant region, which may be derived from a human IgG1 heavy chain constant region.

In the present application, the antigen-binding protein may comprise an antibody light chain constant region. The antibody light chain constant region may comprise an Igκ-derived constant region or an Igλ-derived constant region. The antibody light chain constant region may be derived from a human Igκ constant region.

In the present application, the antigen-binding protein may comprise an antibody or an antigen-binding fragment thereof.

In some embodiments, the antigen-binding fragment may comprise Fab, Fab′, a Fv fragment, F(ab′)2, F(ab)2, scFv, di-scFv, VHH and/or dAb.

In some embodiments, the antibody may comprise a monoclonal antibody. In the present application, the antibody may comprise a murine antibody and/or a chimeric antibody.

In addition, it should be noted that the antigen-binding protein of the present application may comprise a heavy and/or light chain sequence with one or more conserved sequence modifications. The so-called “conserved sequence modifications” refers to amino acid modifications that would not significantly affect or alter the binding properties of an antibody. Such conserved modifications comprise substitutions, additions, and deletions of amino acids. These modifications may be introduced into the antigen-binding protein of the present application by standard techniques known in the art, for example, point mutation and PCR-mediated mutation. Conserved amino acid substitutions refer to substitutions of amino acid residues with those having similar side chains. There are sets of amino acid residues having similar side chains known in the art. These sets of amino acid residues comprise amino acids with basic side chains (for example, lysine, arginine, and histidine), acidic side chains (for example, aspartic acid, and glutamic acid), uncharged polar side chains (for example, glycine, asparagine, glutamine, serine, threonine, tyrosine, cysteine, and tryptophan), non-polar side chains (for example, alanine, valine, leucine, isoleucine, proline, phenylalanine, and methionine), P-branched side chains (for example, threonine, valine, and isoleucine), and aromatic side chains (for example, tyrosine, phenylalanine, tryptophan, and histidine). In some embodiments, one or more amino acid residues in the CDR of the antigen-binding protein of the present application may be substituted with other amino acid residues in the same side chain set. Those skilled in the art know that some conserved sequence modifications do not abolish antigen binding. For details, see, for example, Brummell et al., (1993) Biochem 32:1180-8; de Wildt et al., (1997) Prot. Eng. 10:835-41; Komissarov et al., (1997), J. Biol. Chem. 272:26864-26870; Hall et al., (1992) J. Immunol. 149:1605-12; Kelley and O'Connell (1993) Biochem. 32:6862-35; Adib-Conquy et al., (1998) Int. Immunol. 10:341-6; and Beers et al., (2000) Clin. Can. Res. 6:2835-43.

The antigen-binding protein described herein can be identified, screened, or characterized by various assays known in the art.

For example, the antigen-binding activity of the antigen-binding protein or a fusion protein described in the present application can be tested by known methods such as enzyme-linked immunosorbent assay (ELISA), immunoblotting (e.g., Western blotting), flow cytometry (e.g., FACS), immunohistochemistry, immunofluorescence, etc.

In the present application, the antigen-binding protein may specifically bind to DIR or a functionally active fragment thereof. In the present application, the antigen-binding protein may specifically bind to DIR-II.

In some embodiments, the DIR or a functionally active fragment thereof may be a full-length DIR or a functionally active fragment thereof, or a fragment exerting the functional activity of DIR (for example, it may be DIR-II). In some embodiments, the DIR or a functionally active fragment thereof may be isolated DIR or a functionally active fragment thereof, or a mixture of various forms of DIR or functionally active fragments thereof. For example, the DIR or a functionally active fragment thereof may be human DIR or a functionally active fragment thereof.

In some embodiments, the binding activity of the antigen-binding protein to an antigen (e.g., DIR-II) may be determined by Elisa. For example, the antigen-binding protein may have the capability of binding to the antigen or the functional fragment thereof at a value of about 10 ng/ml or above (e.g., at least 15 ng/ml, at least 20 ng/ml, at least 25 ng/ml, at least 30 ng/ml, at least 35 ng/ml, at least 40 ng/ml, at least 45 ng/ml, at least 50 ng/ml, at least 55 ng/ml, at least 60 ng/ml, at least 65 ng/ml, at least 70 ng/ml, at least 75 ng/ml, at least 80 ng/ml, at least 85 ng/ml, at least 90 ng/ml, at least 95 ng/ml, at least 100 ng/ml, at least 500 ng/ml, at least 1000 ng/ml or above).

### Medicament Screening Method

In another aspect, the present application provides a medicament screening method, which may comprise the step of: detecting changes in a content of DIR and/or of a nucleic acid encoding the DIR in a subject after administration of a candidate medicament, wherein the medicament is for use in prevention, treatment and/or alleviation of cognitive impairment and/or a neurodegenerative disease.

In the present application, when the content of the DIR and/or of the nucleic acid encoding the DIR in the subject is reduced due to administration of the candidate medicament, the candidate medicament has a therapeutic effect.

In the present application, the administration may comprise injection.

In the present application, the medicament may comprise a small molecule medicament and/or a bio-macromolecular medicament.

According to the disclosure in the present application, the DIR level is closely related to the presence and/or degree of cognitive impairment, and thus can be used as an indicator for diagnosis of AD diagnosis and/or evaluation of the progression (e.g., grading or staging) of AD.

In addition, the present application further provides a method for screening of a candidate medicament for treatment of AD. The method comprises: detecting an effect of the candidate medicament on the DIR level in a subject or a sample derived from the subject, wherein after the use of the candidate medicament, a decrease in the DIR level indicates that the candidate medicament has a therapeutic effect on the AD. Each feature involved in the method for screening of a candidate medicament of the present application may be defined or described as in the present application. In some embodiments, the candidate medicament may be a DIR inhibitor.

The present application found that DIR is specifically expressed in blood samples and/or cerebrospinal fluid of AD patients. One important thing is that DIR can be detected in a small amount of serum (such as about 100 μl), which provides a promising detection means for early diagnosis of AD for clinical application. The current results suggest that the positive detection rates of diseases such as AD and MCI are above 90%, and DIR has a great application prospect as a specific marker for early AD screening.

pTau-181, pTau-217, Aβ40 and Aβ42 currently used in clinical blood detection are newly developed detection items for early AD screening. They have relatively low specificity and are expensive. Compared with these existing detections for early AD screening, the early AD screening based on the DIR level in the present application has a high detection rate, very good specificity, and/or low cost, presenting a price advantage. For example, the cost in detection of the DIR level by ELISA may be controlled at less than 10 yuan/person. Therefore, the detection indicators and method of the present application have greater effects and cost advantages compared with existing methods.

In addition, the present application has found that DIR shows good specificity and sensitivity for diagnosis of cognitive impairment and/or a neurodegenerative disease, and can pick out potential AD and/or MCI patients in case of less significant differences among pTau-181, pTau-217, Aβ40, Aβ42 and other targets, allowing for effective diagnosis of patients at an earlier stage.

In the present application, the kit of the present application and/or the detection substance of the present application may be used in the method to detect changes in the content of DIR and/or of a nucleic acid encoding the DIR in a subject before and/or after the administration of the candidate medicament. In the present application, at least one (e.g., 1, 2, 3 or more) detection substance may be used in the method to detect changes in the content of DIR and/or of a nucleic acid encoding the DIR in a subject before and/or after the administration of the candidate medicament. For example, at least one (e.g., 1, 2, 3 or more) antigen-binding protein (e.g., DIR antibody) capable of specifically binding to DIR may be used in the method. In the present application, different antigen-binding proteins existing in the amino acid sequence of at least one CDR may be considered as different species of antigen-binding proteins.

### Information of Sequence Listing:

| SEQ ID NO: | Sequence name | Length | |
|---|---|---|---|
| 92 | Full-length sequence of DDIT4L gene | 4599 | nt |
| 93 | DIR spliced mature mRNA | 4163 | nt |
| 8 | DIR protein encoding molecule | 255 | bp |
| 1 | DIR protein | 84 | aa |
| 94 | DDIT4L protein encoding molecule | 582 | nt |
| 95 | DDIT4L protein | 193 | aa |

The present application provides the embodiment:
1. Application of a substance for detecting a DNA damage-inducible transcript 4-like (DDIT4L) intron retention spliced product (DIR) from a sample of a subject in preparation of a product (e.g., a kit or detection system) for diagnosis of presenile dementia and mild cognitive impairment and/or evaluation of progression (e.g., grading or staging) of the presenile dementia in the subject.
2. The application according to Embodiment 1, wherein the spliced product is selected from the group consisting of: mRNA having a nucleotide sequence as set forth in SEQ ID NO: 93; a peptide-encoding nucleic acid of the spliced product having a nucleotide sequence as set forth in SEQ ID NO: 8; or a peptide of the spliced product having the amino acid sequence as set forth in SEQ ID NO: 1.
3. The application according to Embodiment 1, wherein the presenile dementia comprises: an early stage (an amnesia stage), an intermediate stage (a neurological disorder stage) and a late stage (a severe dementia stage).
4. The application according to Embodiment 1, wherein the substance is selected from a reagent, an apparatus, or their combination for detecting the DIR.
5. The application according to Embodiment 1, wherein the substance is selected from: a substance specific to the DIR, for example, an antibody (preferably, a monoclonal antibody) of the DIR; a probe, gene chip, PCR primer, or gRNA specific to DIR, etc.
6. The application according to Embodiment 1, wherein the sample is selected from: a blood sample, such as whole blood, serum, and plasma; and cerebrospinal fluid.
7. The application according to Embodiment 1, wherein an increase in the DIR level in the sample compared to the normal control value indicates that the subject suffers from the presenile dementia or mild cognitive impairment; or, an increase in the DIR level in the sample compared to a mild cognitive impairment control or an early control level in a subject who has been diagnosed with presenile dementia indicates some development (e.g., a higher grade or stage) of the presenile dementia in the subject.
8. The application according to Embodiment 1, wherein the present application provides a product (e.g., kit or system) for diagnosis of presenile dementia and mild cognitive impairment, and/or for evaluation of progression (e.g., a grade or stage) of the presenile dementia, comprising:
(i) a substance for detecting a DNA damage-inducible transcript 4-like (DDIT4L) intron retention spliced product (DIR) from a sample of a subject; and
(ii) optionally, other substances for diagnosis of the presenile dementia and mild cognitive impairment, and/or for evaluation of progression (e.g., a grade or stage) of the presenile dementia, for example, a substance for detecting an existing marker for the presenile dementia, for example, in some cases:
(i) the substance in (i) is selected from one or more of the group consisting of:
a reagent, an apparatus or their combination for detecting the DIR;
a substance for detecting the DIR at the gene level and/or protein level, such as a substance for use in one or more detection techniques or methods selected from the group consisting of: immunohistochemistry (e.g., immunofluorescence analysis, chemiluminescence, reverse enzyme-linked immunosorbent assay, an immunocolloidal gold method), Western blotting, Northern blotting, PCR, and a biochip method; or
a substance specific to the DIR, for example, an antibody (preferably, a monoclonal antibody) of the substance; a probe, gene chip, PCR primer, or gRNA specific to the spliced product; and/or
(ii) other substances in (ii) are selected from: a substance for detecting one or more other markers for presenile dementia, for example: AD7C-NTP, pTau-181, pTau-217, Aβ40 and Aβ42; and/or, a reagent for imaging of brain amyloid protein; and/or, a reagent for diagnosis of the imaging of the subject's brain.
9. A system for diagnosis of presenile dementia and mild cognitive impairment, and/or for evaluation of progression (e.g., a grade or stage) of the presenile dementia, comprising the following devices:
(a) a device for collecting and/or receiving data on a DIR level in a DNA damage-inducible transcript 4-like (DDIT4L) intron retention spliced product (DIR) from a sample; and
(b) a device for analyzing the data to diagnose presenile dementia and mild cognitive impairment, and/or evaluate the progression (e.g., a grade or stage) of the presenile dementia,
wherein an increase in the DIR level in the sample compared to the normal control value indicates that the subject suffers from the presenile dementia or mild cognitive impairment; or, an increase in the DIR level in the sample compared to a mild cognitive impairment control or an early control level in a subject who has been diagnosed with presenile dementia indicates some development (e.g., a higher grade or stage) of the presenile dementia in the subject.
10. A method for screening of a medicament for treatment of presenile dementia, comprising: detecting an effect of a candidate medicament on the level of a DNA damage-inducible transcript 4-like (DDIT4L) intron retention spliced product (DIR) in a subject, wherein after the use of the candidate medicament, a decreased level indicates that the candidate medicament has a therapeutic effect on the presenile dementia.

Not wishing to be bound by any particular theory, the following examples are merely to set forth the detection method, application or the like of the present application, and are not intended to limit the scope of the invention of the present application.

### EXAMPLES

The present application is further set forth below in conjunction with specific examples. It should be understood that these examples are intended only to explain the present application, instead of limiting the scope of the present application. Those skilled in the art can make appropriate modifications and changes to the present application, and these modifications and changes shall fall within the scope of the present application.

For the experimental methods without specifying specific conditions in the following examples, the conventional methods in the art can be used (for example, see “Molecular Cloning: A Laboratory Manual”, Third Edition, New York: Cold Spring Harbor Laboratory Press, 1989), or as recommended by the supplier. DNA sequencing methods are conventional methods in the art, or tests can also be provided by commercial companies.

Unless otherwise stated, percentages and parts are by weight. Unless otherwise defined, all technical and scientific terms used herein have the same meaning as those familiar to those skilled in the art. In addition, any methods and materials that are similar or equivalent to those described can be applied to the method of the present application. The preferred implementation methods and materials described in herein are for demonstration purposes only.

### Example 1 DIR Protein as Secreted Protein

### Test Materials and Methods

Method for plasmid construction and cell transfection: The cDNAs of human DIR were cloned into pCMV-flag vectors to express flag-DIR proteins. HEK293 cells (Institute of Biochemistry and Cell Biology, Chinese Academy of Sciences) were transfected with pCMV-flag plasmids and pCMV-flag-DIR plasmids using a Lippo3000 reagent; after being cultured for 48 hours, the cells and medium were harvested, and an appropriate amount of lysate was added; the resulting mixture was rotated for 30 min at 4° C. for lysis, and centrifuged for 30 min at 12,000 g to obtain the supernatant; and a small amount of lysate was taken for Western Blot analysis.

Western Blot experiment: It was completed following the steps of SDS-PAGE electrophoresis, membrane transfer, immunoreaction and chemiluminescence (the antibodies used were purchased from GL Biochem (Shanghai) Ltd.).

### Results and Discussions

After the HEK293 cells were cultured for 48 hours, a large amount of DIR was expressed in the cells transfected with the pCMV-flag-DIR plasmids, and a large amount of DIR was also detected in the medium for the cells (FIG. 1B). This indicates that DIR can be secreted into the extracellular medium through the intracellular secretion pathway after its transcription and translation into proteins. In terms of the amount of secretion, part of the intracellularly synthesized DIR is secreted into the extracellular medium, suggesting that DIR may also be secreted to outside of cells in a human body and then enter the blood circulation system. This provides a theoretical basis for taking DIR as a blood marker under some pathological conditions.

### Example 2 Changes in Content of DIR Proteins in Blood of AD Patients

### Test Materials and Methods

Tissue samples: Venous blood samples (58 samples in total) from AD and MCI patients, non-AD patients (patients with hypertension, coronary heart disease or diabetes) and normal people were sourced from Huashan Hospital Affiliated to Fudan University and Xuhui Central Hospital.

Methods for collecting venous blood and obtaining plasma: The blood was collected from the vein in cubital fossa of a patient into an EDTA vacuum tube by using the vacuum blood collection technique, and the tube was immediately inverted gently 8 times to fully mix the blood and an anticoagulant. Then, the tube was transported to the laboratory on ice, centrifuged for 10 min at 1500 g at 4° C., and the supernatant was aspirated, aliquoted, and stored at −80° C.

Western Blot method for plasma: The aliquoted plasma was thawed on ice and diluted appropriately with PBS, followed by the addition of the buffer for denaturation for 5 minutes in a 100° C. metal bath. The subsequent steps were the same as the Western Blot method for tissues.

ELISA method for plasma: The plasma sample was diluted at 1:1 with 1× diluent (OBT1998G, purchased from Bio-Rad); then, 100 μl of a blocking solution was added using a pipette for blockage for 1 h at 37° C.; then, 100 μl of a proportionally diluted standard and diluted plasma were added into plate wells coated with capture antibodies (GL Biochem, AB009458) by using the pipette, the tip of which was changed for each sampling; the resulting mixture was incubated for 1.5 h at 37° C.; the liquid in each well was removed, and the well was washed 3 times with 100 μl 1× washing solution (BUF031C, purchased from Bio-Rad); 100 μl of detection antibody (i. e., the DIR antibody IR-II-6, of which a VH amino acid sequence was as set forth in SEQ ID NO. 58, and of which a VL amino acid sequence was as set forth in SEQ ID NO. 60) was added to each well and incubated for 1 h at 37° C.; the liquid in each well was removed, and the well was washed 3 times with 100 μl 1× washing solution; 100 μl of 1× avidin-HRP solution was added to each well, which was incubated for 1 h at 37° C.; the liquid in each well was removed, and the well was washed 3 times with 100 μl 1× washing solution; 100 μl of 1× substrate reaction solution (BUF062C, purchased from Bio-Rad) was added to each well, which was incubated for 10-30 min at room temperature in the dark; and a microplate reader was used for detection at a wavelength of 655 nm, and a standard curve was established.

### Results and Discussions

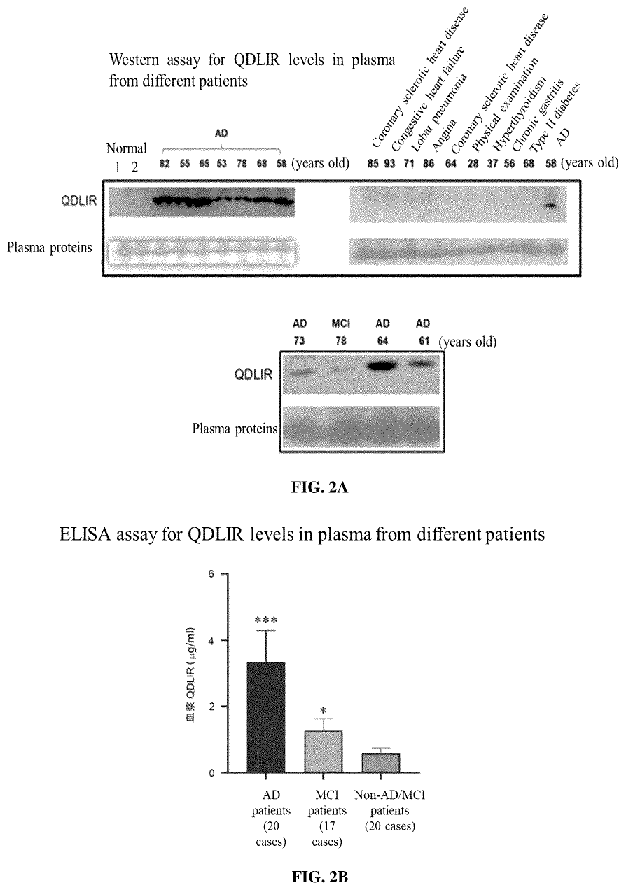

Based on the testing of different patients by clinical collection of blood samples from AD and MCI patients, non-AD patients and normal people, it was found that the content of DIR in the blood samples from the AD and MCI patients was the highest, and it was detectable in about 95% of patients; the blood samples from the MCI patients also contained a relatively high amount of DIR, and it was detectable in about 90% of patients; and in the blood samples from patients without cognitive impairment, there was no or very small amount of DIR detected, and no DIR was detected in the blood samples from the normal people (FIG. 2A).

Through the ELISA method, it was found that the content of DIR in the blood samples from the AD patients was relatively high, reaching a level of 10-100 μg/ml, while the content of DIR in the non-AD patients was at the pg/ml level and was undetected in the normal people. Therefore, the DIR level in the AD patient was 103-104 times higher than that of other patients. This suggests very high specificity of DIR (FIG. 2A and FIG. 2B).

The above results indicate that DIR has a high concentration in the blood of the AD and MCI patients and can be used as a blood marker to detect the presence of AD, in particular for patients with mild cognitive impairment.

Moreover, the content of DIR is related to the severity of AD (see FIG. 2B).

### Example 3 Methodological Study on DIR Protein Detection

The DIR protein (GL Biochem, 752084) was prepared into a 1 mg/ml stock solution with a DPBS solution, which was diluted with an ELISA washing solution (BUF031C, purchased from Bio-Rad) before the experiment to prepare a standard DIR protein solution.

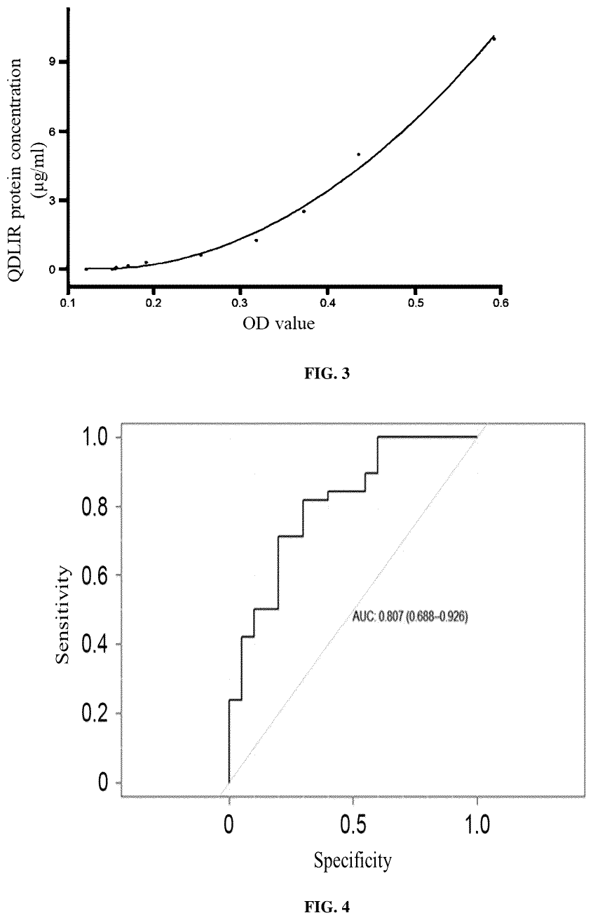

The concentration of standard DIR protein was detected by ELISA, and polynomial regression was used for fitting. The resulting standard curve was shown in FIG. 3.

The R² value of this regression model was 0.99, indicating a high degree of fitting; the shaded part showed the 95% confidence interval of the regression model; and as can be seen from the figure, the confidence interval was narrow, indicating high confidence level of the DIR content in the sample as estimated based on this model.

### Example 4 ROC Curve with DIR as Clinical Detection Indicator

The blood samples clinically collected from AD and MCI patients, non-AD patients and normal people (same as Example 2) were detected by ELISA; and based on the detection results, a logistic regression model was established from the achieved DIR concentration using the “pROC” package analysis of R software (Version 3.6.2); and the self-service sampling method was used for analysis to obtain the ROC curve.

The results were shown in FIG. 4. The results show that good prediction results can be achieved by modeling using the single parameter of DIR only, with AUC=0.807 (95% confidence interval: 0.688-0.926).

### Example 5 Changes in Content of DIR Protein in Blood of Patients with Different Types of MCI

### Test Materials and Methods

Tissue samples: Venous blood samples from patients with different types of MCI and normal follow-up people were sourced from Huashan Hospital Affiliated to Fudan University.

Methods for collecting venous blood and obtaining plasma: Same as Example 2.

ELISA method for plasma: Same as Example 2.

MCI classification method: aMCI-s: single-domain amnestic MCI; aMCI-m: multi-domain amnestic MCI; naMCI-s: single-domain non-amnestic MCI; and naMCI-m: multi-domain non-amnestic MCI.

In Example 5, there were 122 samples from aMCI-m type patients; 85 samples from aMCI-s type patients; 11 samples from naMCI-m type patients; 88 samples from naMCI-s type patients; and 279 samples from normal follow-up people.

### Results and Discussions

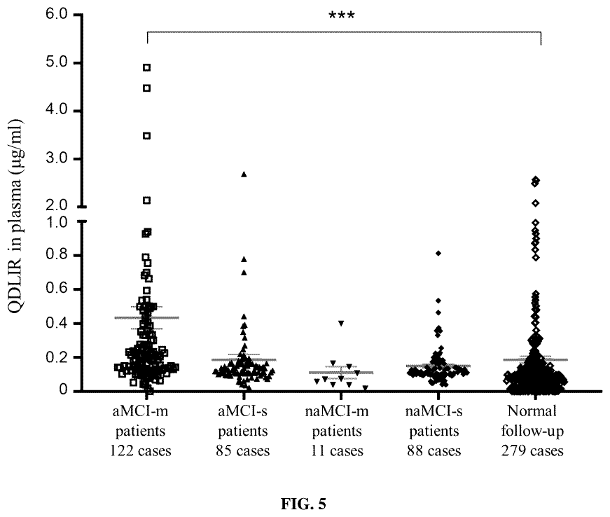

Based on the detection of the blood samples clinically collected from patients with different types of MCI and normal follow-up people, it was found that the content of DIR in the blood samples of MCI patients with aMCI-m type was increased, exhibiting a significant difference compared with the normal follow-up people (FIG. 5).

Through the ELISA method, it was found that the content of DIR in the blood of aMCI-m type patients was high, and the aMCI-m type patients were more likely to progress to AD than patients with other types of MCI, suggesting that DIR was an indicator capable of distinguishing aMCI-m types.

The above results indicate that DIR can be used as an effective diagnostic marker for AD.

### Example 6 Changes in Content of DIR Protein in Blood of Patients with Different Types of MCI

### Test Materials and Methods

Tissue samples: Venous blood samples from patients with different types of MCI, AD patients and normal follow-up people were sourced from Huashan Hospital Affiliated to Fudan University.

Methods for collecting venous blood and obtaining plasma: Same as Example 2.

ELISA method for plasma: The method for DIR was the same as that in Example 2; and Tau, Aβ40 and Aβ42 were subjected to commission detection by the triple kit (Product No.: 101995) of Hangzhou G-Bio Co., Ltd. according to its operating procedures.

MCI classification method: Same as Example 5.

### Results and Discussions

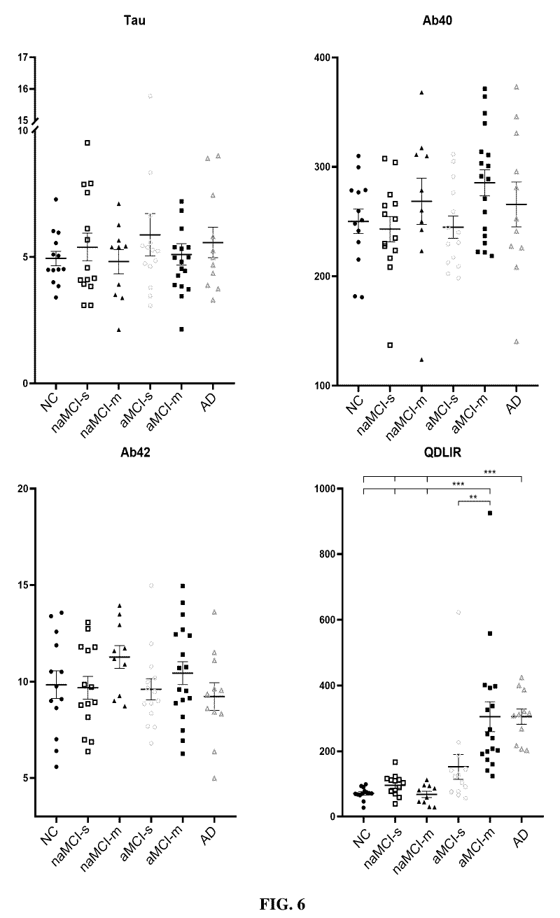

Based on our detection of the blood samples clinically collected from patients with different types of MCI, AD patients and normal follow-up people, it was found that the content of DIR in the blood samples from aMCI-m type patients and AD patients was increased, exhibiting a significant difference compared with the normal follow-up people and the patients with other MCI types; and the existing detection of indicators such as Tau, Aβ40 and Aβ42 in blood samples showed no significant different between the groups, although some indicators (such as Aβ40) showed the tendency to increase in the blood from aMCI-m type patients and AD patients (see FIG. 6).

Through the ELISA method, it was found that the content of DIR in the blood from aMCI-m type patients and AD patients was higher. It can be seen that compared with the existing auxiliary diagnostic indicators Tau, Aβ40 and Aβ42, DIR can more clearly distinguish the aMCI-m type patients, the AD patients and other types of MCI patients or normal people in the early stage. Therefore, DIR can be used as an effective diagnostic marker for aMCI-m and AD.

### Example 7 Changes in Content of DIR Proteins in Blood and Cerebrospinal Fluid from Patients

### Test Materials and Methods

Tissue samples: Venous blood samples and cerebrospinal fluid samples from the same patient (one case for each of cerebral edema, meningioma and glioma) were both sourced from Huashan Hospital Affiliated to Fudan University.

Methods for collecting venous blood and obtaining plasma: Same as Example 2.

Cerebrospinal fluid collection method: The cerebrospinal fluid was obtained from the patient during surgery.

ELISA method for plasma and cerebrospinal fluid: Same as Example 2.

### Results and Discussions

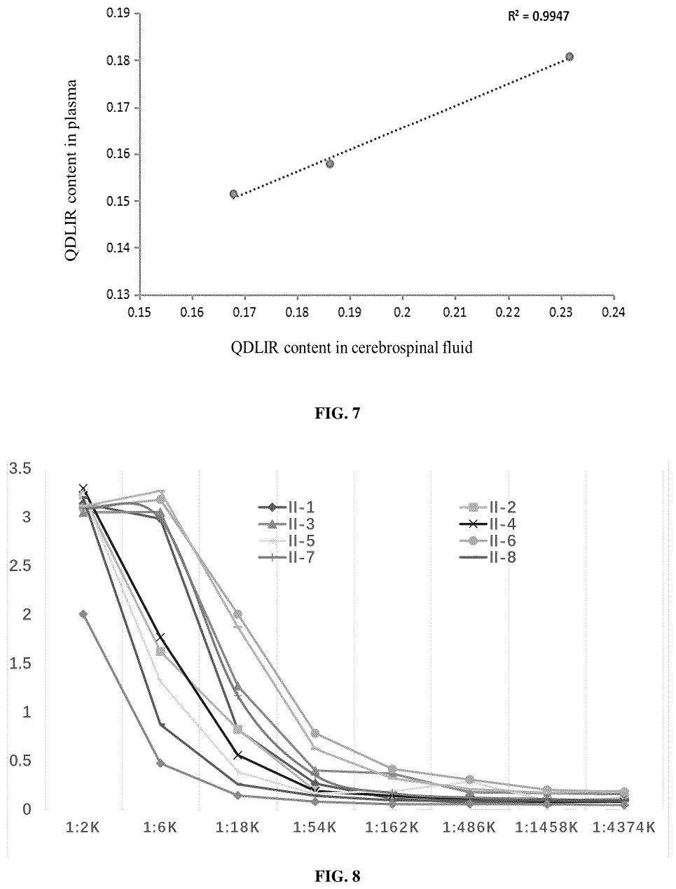

The content of DIR in the plasma and in the cerebrospinal fluid was highly consistent, with an R² value of 0.99 (see FIG. 7).

Through the ELISA method, the content of DIR in the blood from the patient was consistent with that in the cerebrospinal fluid, indicating that the content detected in the blood could directly reflect the content of DIR in the cerebrospinal fluid, suggesting that the DIR described in the present application is also suitable for use as an effective diagnostic marker in the case of taking the cerebrospinal fluid as a sample.

### Example 8 Detection of Binding Activity of DIR Antibodies

The sequencing results of DIR antibodies IR-II-1 to IR-II-10 (IR-II-10 was A2D10) were shown in Table 1 below.

TABLE 1

| | HCDR1 | HCDR2 | HCDR3 | LCDR1 | LCDR2 | LCDR3 | VH | VL |
|---|---|---|---|---|---|---|---|---|
| IR-II-1 | SEQ ID | SEQ ID | SEQ ID | SEQ ID | SEQ ID | SEQ ID | SEQ ID | SEQ ID |
| | NO. 20 | NO. 21 | NO. 22 | NO. 25 | NO. 26 | NO. 27 | NO. 23 | NO. 28 |
| IR-II-2 | SEQ ID | SEQ ID | SEQ ID | SEQ ID | SEQ ID | SEQ ID | SEQ ID | SEQ ID |
| | NO. 20 | NO. 21 | NO. 30 | NO. 25 | NO. 26 | NO. 27 | NO. 31 | NO. 33 |
| IR-II-3 | SEQ ID | SEQ ID | SEQ ID | SEQ ID | SEQ ID | SEQ ID | SEQ ID | SEQ ID |
| | NO. 35 | NO. 36 | NO. 37 | NO. 25 | NO. 26 | NO. 27 | NO. 38 | NO. 40 |
| IR-II-4 | SEQ ID | SEQ ID | SEQ ID | SEQ ID | SEQ ID | SEQ ID | SEQ ID | SEQ ID |
| | NO. 20 | NO. 42 | NO. 43 | NO. 25 | NO. 26 | NO. 27 | NO. 44 | NO. 46 |
| IR-II-5 | SEQ ID | SEQ ID | SEQ ID | SEQ ID | SEQ ID | SEQ ID | SEQ ID | SEQ ID |
| | NO. 20 | NO. 48 | NO. 49 | NO. 52 | NO. 26 | NO. 53 | NO. 50 | NO. 54 |
| IR-II-6 | SEQ ID | SEQ ID | SEQ ID | SEQ ID | SEQ ID | SEQ ID | SEQ ID | SEQ ID |
| | NO. 56 | NO. 36 | NO. 57 | NO. 25 | NO. 26 | NO. 27 | NO. 58 | NO. 60 |
| IR-II-7 | SEQ ID | SEQ ID | SEQ ID | SEQ ID | SEQ ID | SEQ ID | SEQ ID | SEQ ID |
| | NO. 35 | NO. 36 | NO. 62 | NO. 25 | NO. 26 | NO. 27 | NO. 63 | NO. 60 |
| IR-II-8 | SEQ ID | SEQ ID | SEQ ID | SEQ ID | SEQ ID | SEQ ID | SEQ ID | SEQ ID |
| | NO. 35 | NO. 42 | NO. 37 | NO. 52 | NO. 26 | NO. 53 | NO. 65 | NO. 54 |
| IR-II-9 | SEQ ID | SEQ ID | SEQ ID | SEQ ID | SEQ ID | SEQ ID | SEQ ID | SEQ ID |
| | NO. 56 | NO. 36 | NO. 57 | NO. 52 | NO. 26 | NO. 67 | NO. 58 | NO. 68 |
| A2D10 | SEQ ID | SEQ ID | SEQ ID | SEQ ID | SEQ ID | SEQ ID | SEQ ID | SEQ ID |
| | NO. 10 | NO. 11 | NO. 12 | NO. 15 | NO. 16 | NO. 17 | NO. 13 | NO. 18 |

The binding activity of the above antibodies IR-II-1 to IR-II-10 to fragments DIR-II was detected. The method specifically comprises:

The target fragment DIR-II was coated on a 96-well plate using a coating buffer and blocked with a blocking buffer. Next, the monoclonal antibodies (1 mg/mL) were diluted in equal proportions (1:2k, 1:6k, 1:18k, 1:54k, 1:162k, 1:486k, 1:1458k, 1:4374k), added to single wells using a pipette, shaken evenly for 2 h at 37° C., and washed three times with a washing buffer. Then, a secondary antibody with horseradish peroxidase (1:5k) was added and incubated at 37° C. After washing three times, an appropriate volume of TMB substrate solution was added, and the resulting mixture was incubated at 37° C. for a certain period of time, and then placed under a microplate reader to record the absorbance value of the sample at a wavelength of 450 nm.

The results were shown in Table 2 and FIG. 8. From these results, it can be seen that the above antibodies IR-II-1 to JR-II-10 specifically bind to the fragments DIR-II at a working concentration of about 10 ng/ml or above. Therefore, all the above antibodies JR-II-1 to JR-II-10 can be used for the detection of DIR proteins and the diagnosis of aMCI-m and AD.

TABLE 2

| | | | | | | | | | | IR-II-10 |
|---|---|---|---|---|---|---|---|---|---|---|
| Valence | IR-II-1 | IR-II-2 | IR-II-3 | IR-II-4 | IR-II-5 | IR-II-6 | IR-II-7 | IR-II-8 | IR-II-9 | (A2D10) |
| 1:2k | 3.1394 | 3.229 | 3.0488 | 3.2953 | 3.2435 | 3.0937 | 3.094 | 3.1999 | 3.112 | 2.0055 |
| 1:6k | 2.9827 | 1.6224 | 3.052 | 1.7675 | 1.313 | 3.1805 | 2.9775 | 0.8698 | 3.2696 | 0.4725 |
| 1:18k | 0.8184 | 0.823 | 1.2676 | 0.5554 | 0.3867 | 2.0055 | 1.1649 | 0.2571 | 1.8719 | 0.1422 |
| 1:54k | 0.2637 | 0.193 | 0.3995 | 0.1857 | 0.1585 | 0.7798 | 0.3497 | 0.138 | 0.6219 | 0.0783 |
| 1:162k | 0.1092 | 0.1398 | 0.3663 | 0.1361 | 0.1898 | 0.4141 | 0.1641 | 0.0911 | 0.3182 | 0.0517 |
| 1:486k | 0.0656 | 0.1116 | 0.1734 | 0.0955 | 0.2848 | 0.3057 | 0.1207 | 0.0864 | 0.2072 | 0.0517 |
| 1:1458k | 0.0653 | 0.0839 | 0.1657 | 0.0751 | 0.0911 | 0.2004 | 0.1034 | 0.0906 | 0.1692 | 0.0501 |
| 1:4374k | 0.0934 | 0.1149 | 0.1532 | 0.0775 | 0.0868 | 0.18 | 0.0867 | 0.0887 | 0.1931 | 0.0427 |

### Example 9 DIR RNA Level in AD Patients

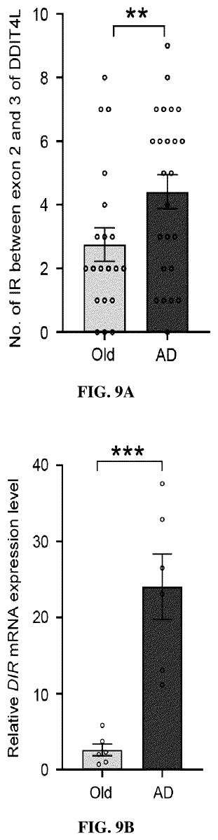

By IRfinder (For IRfinder, see Ngian, Z. K. et al. Truncated Tau caused by intron retention is enriched in Alzheimer's disease cortex and exhibits altered biochemical properties. Proc Natl Acad Sci USA 119, e2204179119 (2022).), it is found that RNA level of DIR was significantly elevated in the brain tissue of AD patients. For using IRFinder, a reference using ‘IRFinder BuildRef’ command based on the Release 110 of Ensembl human genome GRCh38 gene annotations was build, including RNA.SpikeIn.ERCC.fasta.gz and Human_hg38_nonPolyA_ROI.bed files obtained from IRFinder repository (on the website of github.com/RitchieLabIGH/IRFinder). After downloading the transcriptome FASTQ files as described in the previous subsection, the intron retention via ‘IRFinder FastQ’ command was quantified. The values of ‘SpliceExact’ column as a measure for intron events was used (FIG. 9A).

RT-PCR was performed with the hippocampal tissues from control and AD patients, and found that DIR RNA level increased in AD patients (FIG. 9B). The human brain samples collection was conducted with the approval of the Ethics Committee of Huashan Hospital of Fudan University. Informed consent was obtained from all subjects involved in this study. In detail, the cDNA of patients (L1002 and G2913) was used for the detection of hDDIT4L mRNA by using the forward primer: 5′-TGCTGGACTGTGGCTATCAC-3′ (SEQ ID NO: 96) and the reverse primer: 5′-ACAAGGACCTTTGAGCAACCA-3′ (SEQ ID NO: 97).

### Example 10 Generation of Homozygous DIR-KI Mice

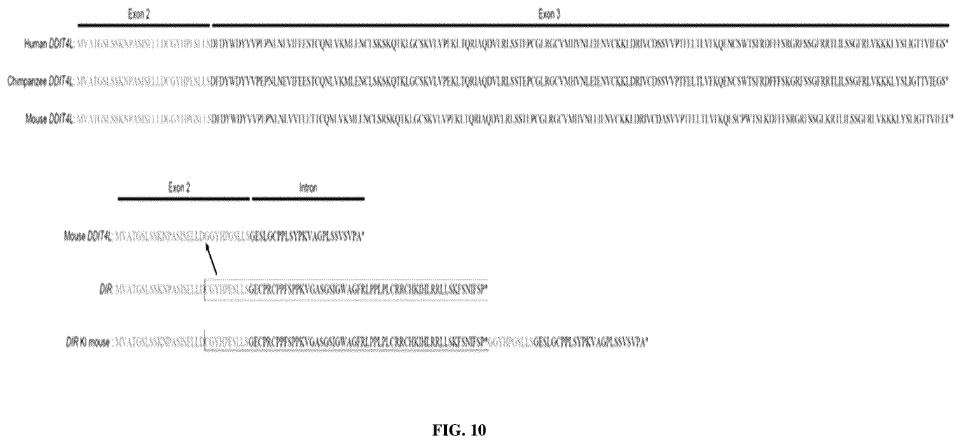

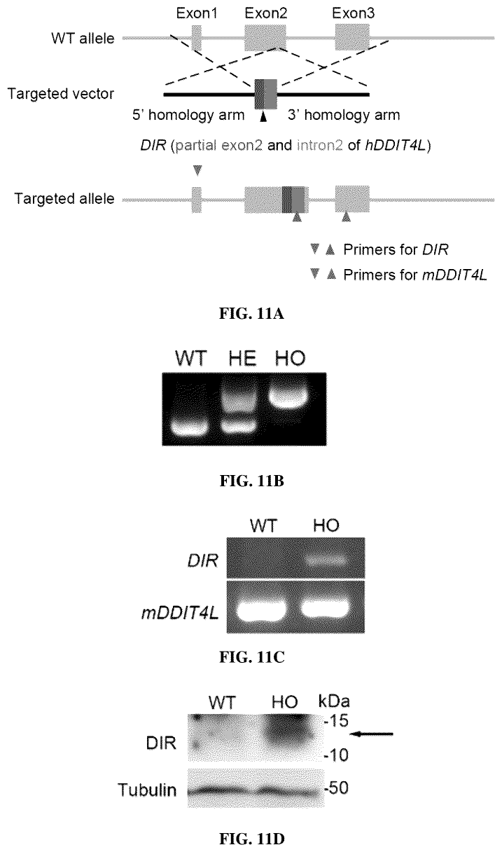

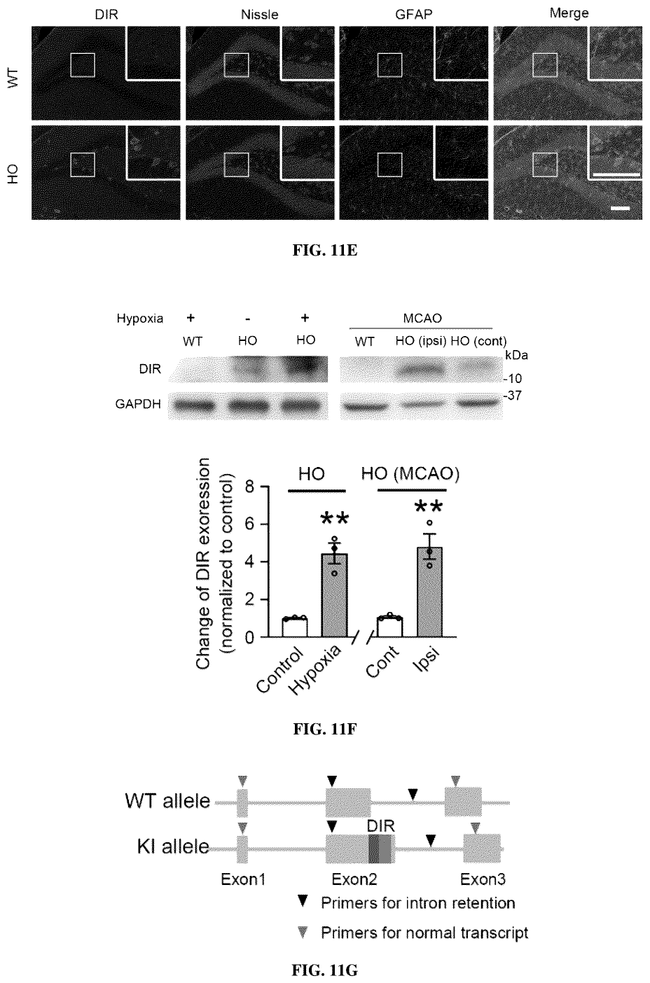

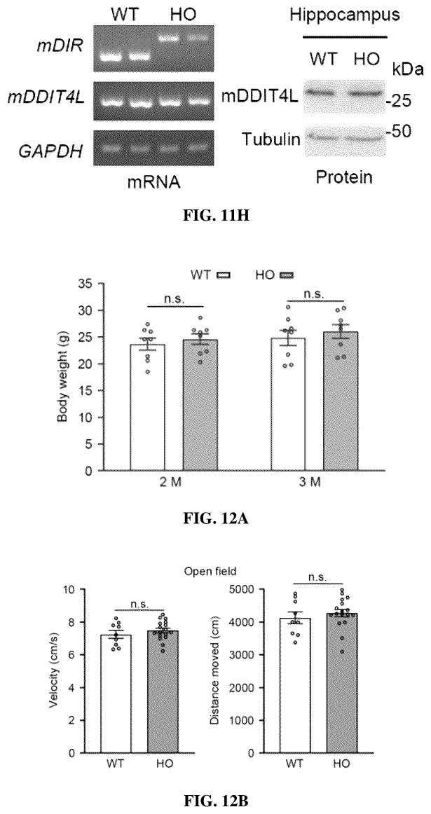

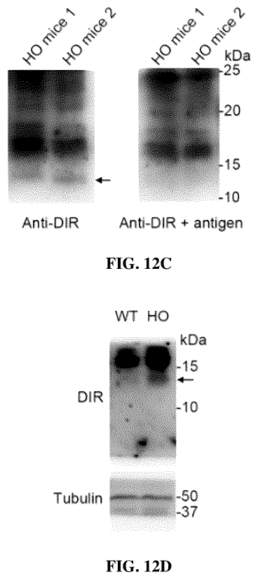

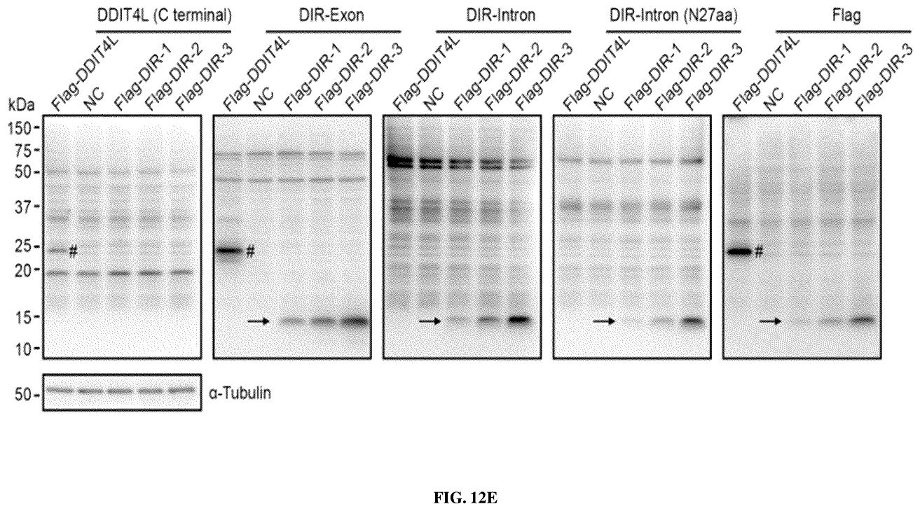

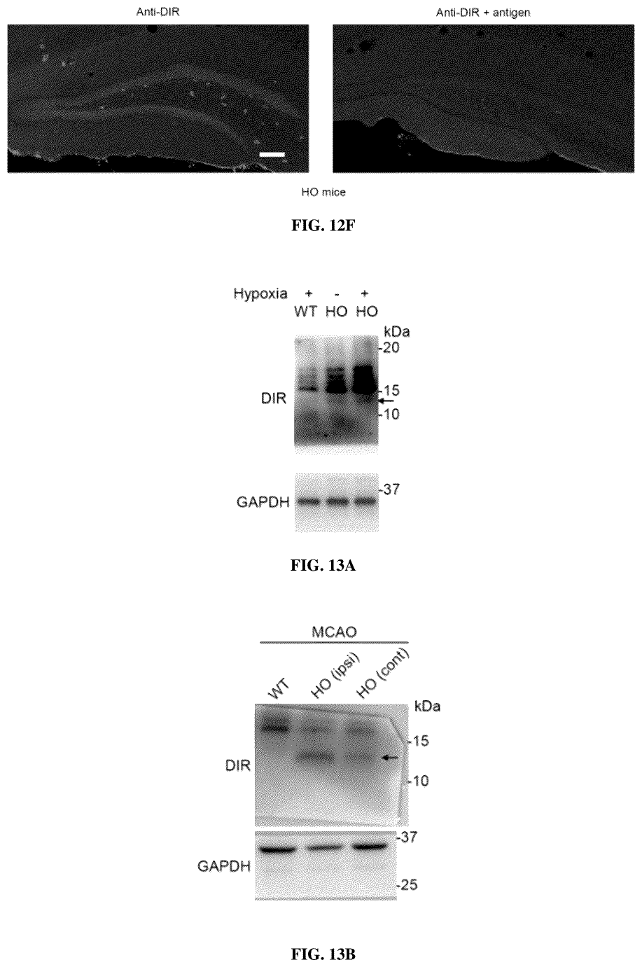

The distribution and function of DIR was investigated in AD by generating DIR-KI mice using CRISPR/Cas9 technology. The DDIT4L protein is conserved in many species (FIG. 1A, FIG. 10). The amino acid (AA) sequences of human and mouse DDIT4L were compared and found that AAs 1-20 were identical and 2 AAs among AAs 21-30, corresponding to exon 2, were different, with the most marked difference being in the intronic region (FIG. 10). To induce the expression of human DIR, the sequence of the human DIR gene encoding the last 64 AAs after the sequence of the mouse DDIT4L gene encoding AAs 1-20 (FIG. 11A; FIG. 10) was inserted. Genotyping and sequencing of newborn mice confirmed accurate sequence insertion (FIG. 11B). Of note, human DIR mRNA was amplified by designed primers (FIG. 11C). The body weight of the homozygous DIR-KI mice was not significantly different from that of the wild-type (WT) mice, suggesting that the mutant mice had normal growth and metabolism (FIG. 12A). Moreover, the WT and homozygous DIR-KI mice showed no difference in velocity or distance travel in the open field test, indicating that motor function was not altered (FIG. 12B).

Western blotting with an antibody against the IR portion of DIR showed the presence of one specific band (˜12 kDa) in the hippocampi of homozygous DIR-KI mice (FIG. 11D, FIG. 12C-12E). Immunostaining of brain slices from homozygous DIR-KI mice showed that DIR was expressed in many hippocampal dentate gyrus (DG) neurons (FIG. 11E; Supplementary FIG. 12F), some neurons in the CA1 and CA3 areas, and a few cortical and thalamic neurons. Therefore, the homozygous DIR-KI mice exhibited DIR protein expression in the hippocampus.

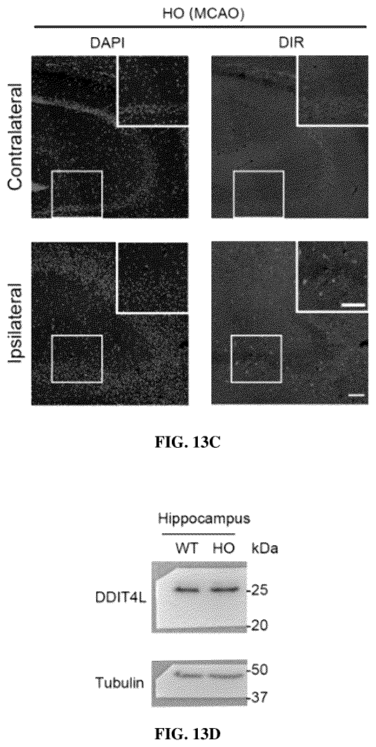

DIR expression in homozygous DIR-KI mice was assessed under hypoxic conditions. The expression of DIR was markedly enhanced in the hippocampi of homozygous DIR-KI mice exposed to low-oxygen conditions (8% O₂) for 8 h (FIG. 11F; FIG. 13A). Unilateral middle cerebral artery occlusion (MCAO) was used to mimic hypoxic-ischemic brain injury, and we found that DIR expression was apparently increased in the ipsilateral hippocampi in homozygous DIR-KI mice subjected to MCAO but not in WT mice subjected to MCAO (FIG. 11F; FIG. 13B and FIG. 13C). Thus, hypoxia could significantly enhance DIR expression in vivo.

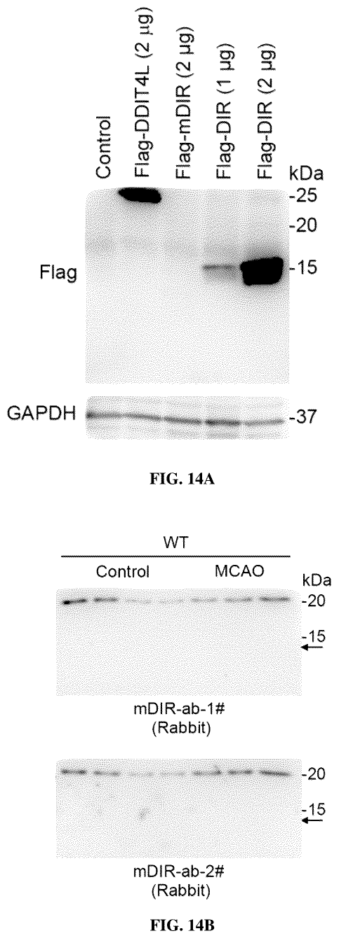

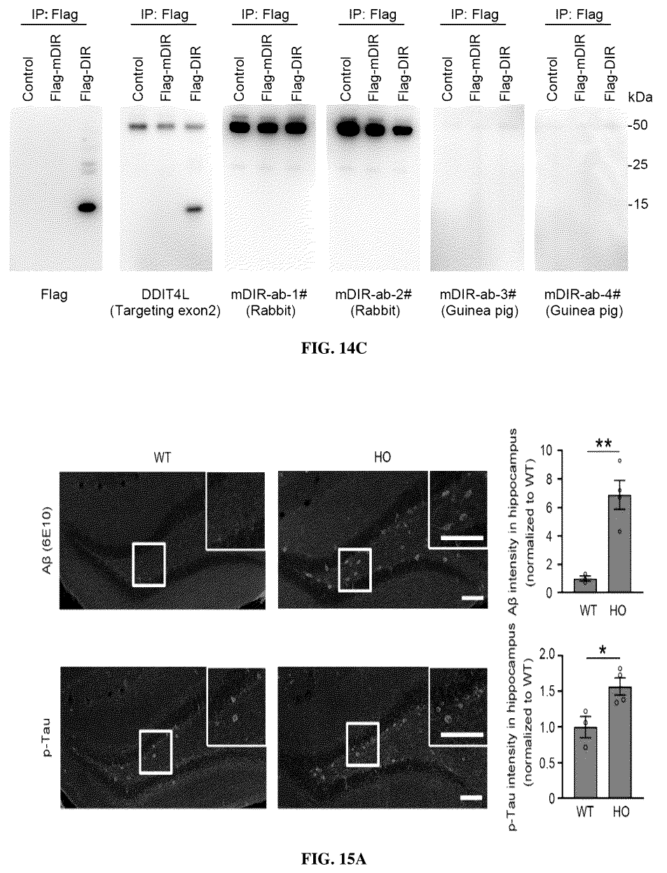

To test whether the mouse intrinsic intron of DDIT4L could be retained, we designed specific primers to clone the fragment from mouse brain mRNA (FIG. 11G). Subsequent sequencing showed the existence of mouse DDIT4L intron retention (mDIR) mRNA in WT mice (FIG. 11H). The mDIR could not be transcribed in homozygous DIR-KI mice and not be translated in flag-mDIR transfect HEK293 cells and WT mice, which may be determined by different spliceosome function in species, while mDDIT4L expression was not changed in homozygous DIR-KI mice, as compared to that in WT mice in normal condition (FIG. 11H, FIG. 13D, FIGS. 14A-14C).

### Example 11 AD-Like Pathological Features in Homozygous DIR-KI Mice

To further investigate the potential roles of DIR in AD-like pathology, we performed immunostaining for Aβ and phosphorylated Tau (p-Tau), two classic AD-related molecules, in brain slices from 3-, 6-, and 9-month-old homozygous DIR-KI mice.

All animal operations were under the guidelines of the National Center for Protein Science Shanghai. Mice were accommodated in the specific pathogen-free rooms and supplied with enough foods and water. The DIR-KI mice were generated by Shanghai Model Organisms Center, Inc. Briefly, a cDNA encoding human DIR was inserted into mouse DDIT4L gene locus (FIG. 11A, FIG. 10) through homologous recombination by using CRISPR/Cas9 technology. All mice were maintained on a C57BL/6J background. The neonatal mice were identified by the PCR analysis, using the following genotyping primers of SEQ ID NO: 98 and 99.

The adult mice were anaesthetized and sacrificed. Then the brain tissues were isolated and homogenized Next, total mRNAs were extracted using Trizol regents and 1 μg mRNAs were transcribed reversely to cDNA. The DIR mRNA, mDIR mRNA, mDDIT4L mRNA and GAPDH mRNA were detected by using the primers of SEQ ID NO: 100-107 respectively.

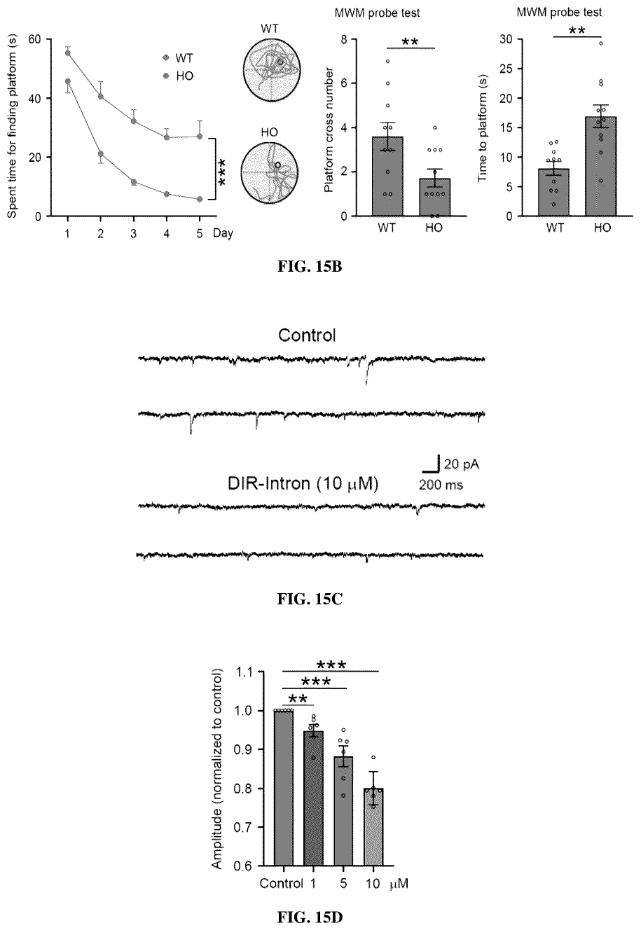

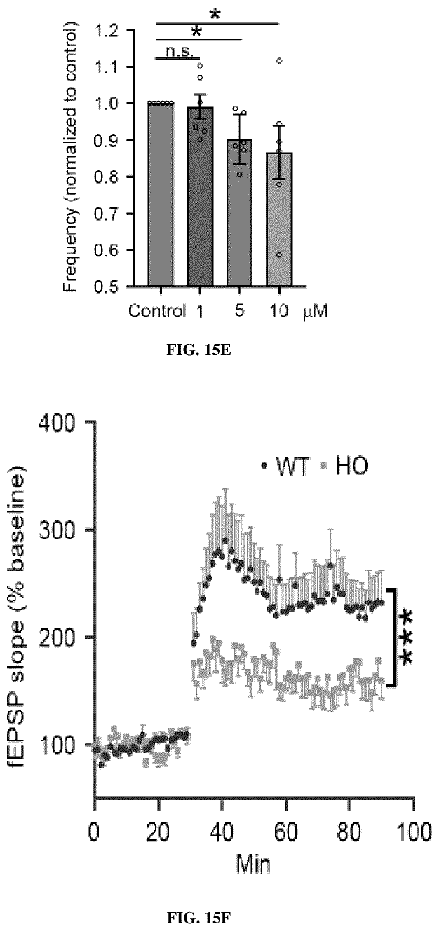

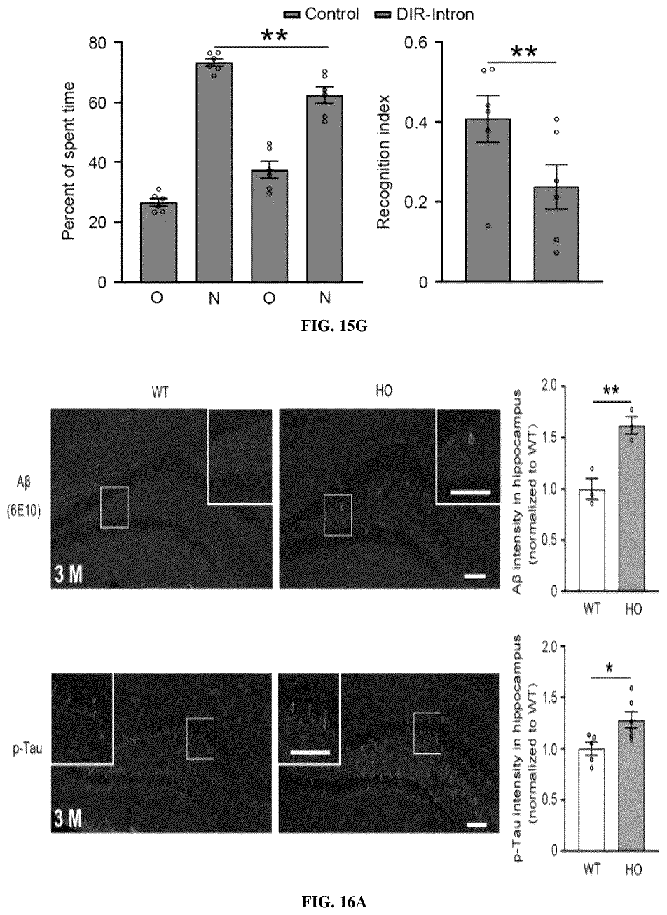

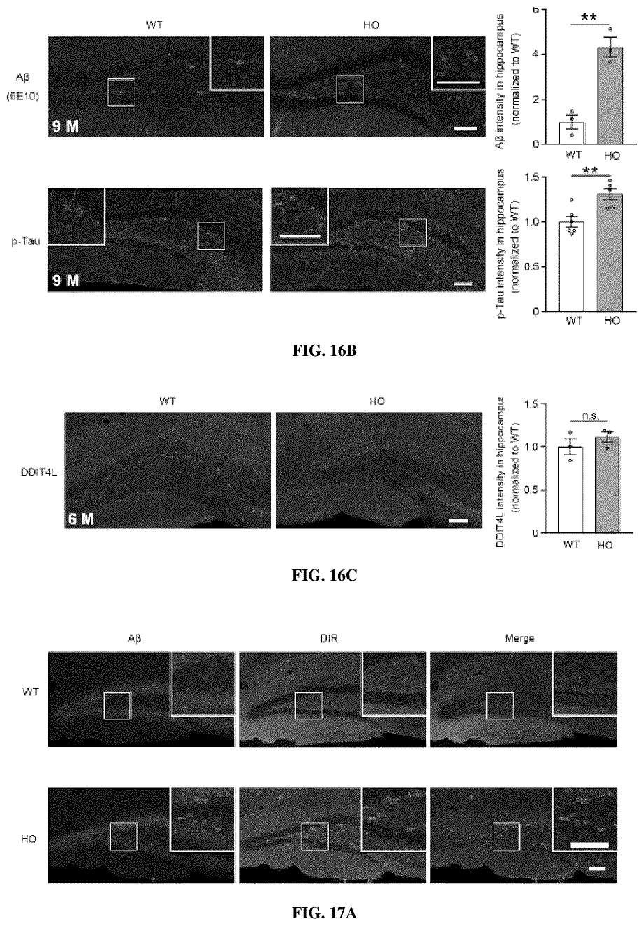

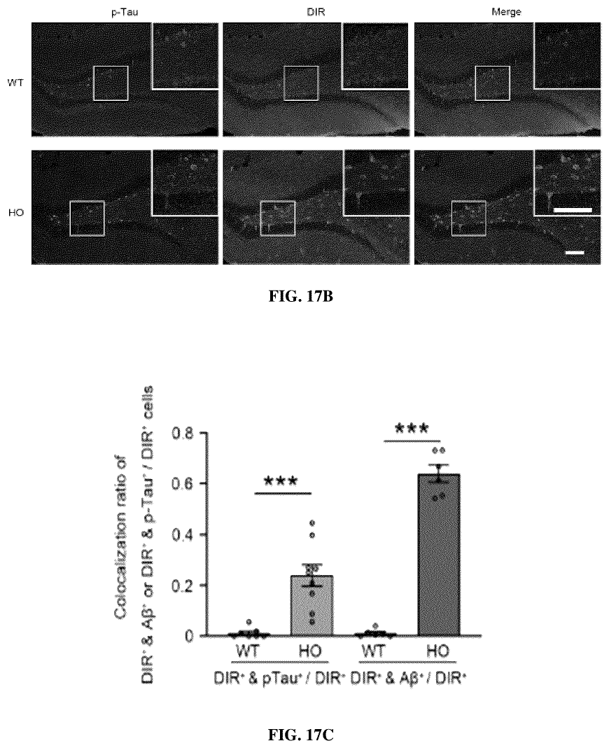

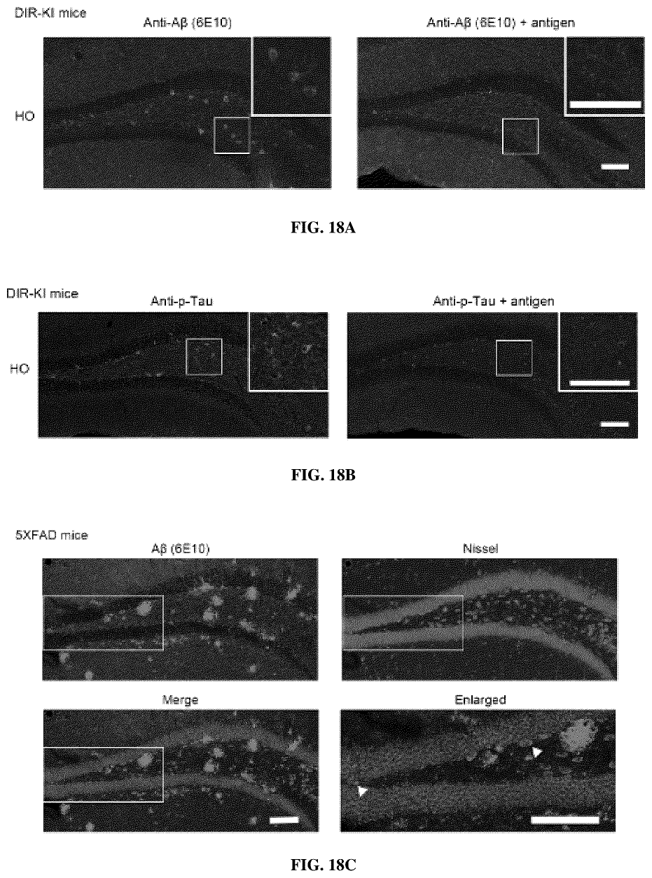

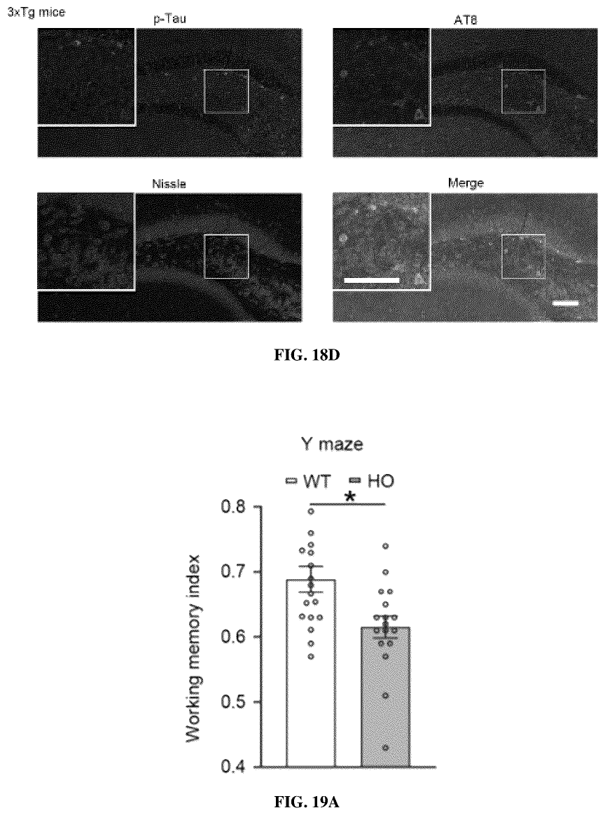

The expression of Aβ and p-Tau was apparently upregulated in the hippocampi of homozygous DIR-KI mice (FIG. 15A; FIG. 16A-16B), while DDIT4L expression showed no change in homozygous DIR-KI and WT mice (FIG. 16C). Importantly, the DIR was partly co-localized with Aβ and p-Tau in hippocampal neurons of homozygous DIR-KI mice (FIG. 17). To check the specificity of Aβ (6E10) and p-Tau antibodies, we did the antigen absorption with Aβ42 and corresponding p-Tau peptide, respectively. The immunostaining results showed the signal of Aβ (6E10) and p-Tau antibody disappeared in the hippocampus of homozygous DIR-KI mice after the antigen absorption (FIG. 18A-18B). We also examined the expression pattern of Aβ (6E10) and p-Tau in the hippocampus of 5×FAD and 3×Tg mice, respectively. Immunostaining results revealed that Aβ (6E10) was expressed in hippocampal neurons and accumulated at plaques deposited in the hippocampus of 5×FAD mice while p-Tau could be detected in 3×Tg mice. (FIG. 18C-18D). Taken together, these results indicate that DIR may promote the progression of AD-like pathology, particularly in the hippocampus.

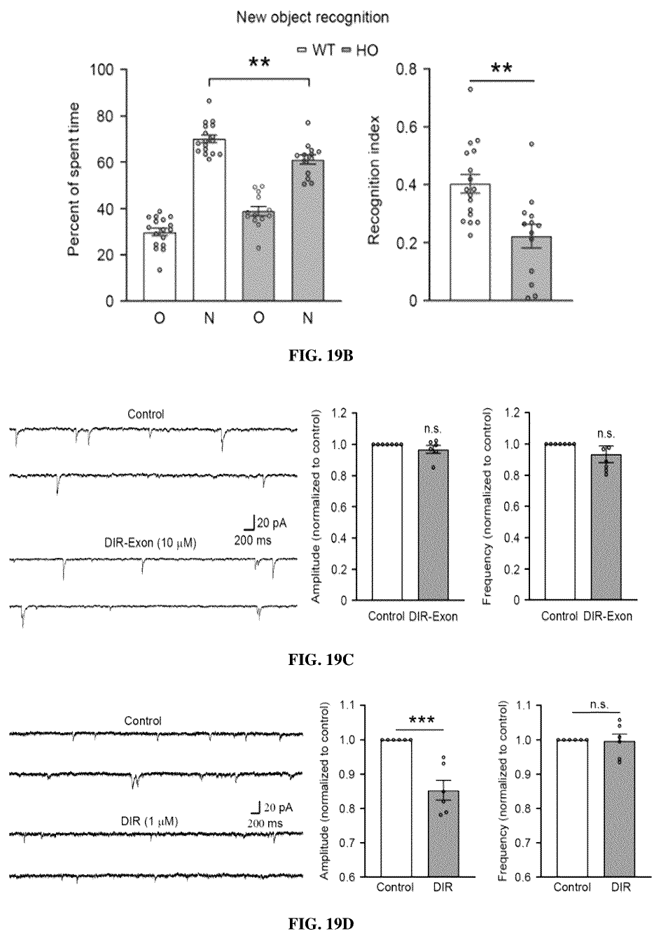

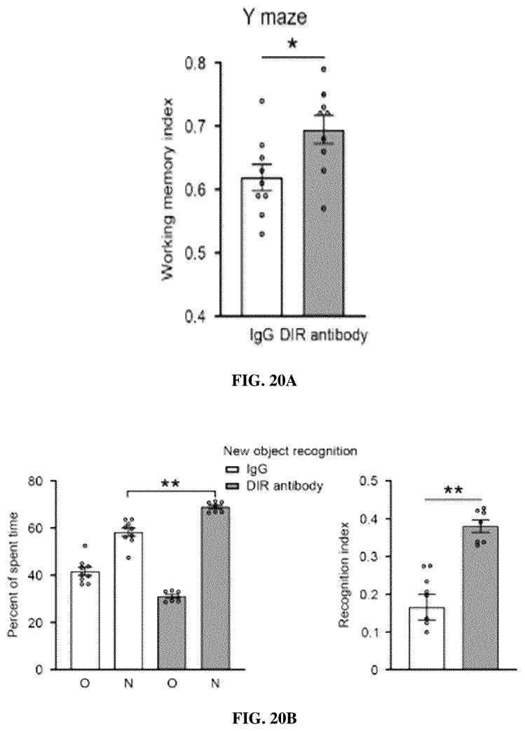

Next, we explored whether cognitive functions were impaired in homozygous DIR-KI mice. The results of the Y maze test showed that working memory was impaired in the homozygous DIR-KI mice (FIG. 19A). Novel object recognition was attenuated in the homozygous DIR-KI mice (FIG. 19B). In the Morris water maze test, most WT mice learned to use distal cues to navigate a direct path to the hidden platform following a 5-day training period. The homozygous DIR-KI mice showed an increased escape latency during the training process (FIG. 15B). In the probe test, which was used to assess spatial memory, the homozygous DIR-KI mice did not show a preference for the target quadrant, whereas WT mice showed a shorter latency to enter the target quadrant and crossed the platform more times (FIG. 15B).

To determine the functional domain of DIR, two polypeptides consisting of the first 30 AAs (DIR-Exon) or the last 54 AAs (DIR-Intron) of DIR were synthesized. Hippocampal slices from WT mice were incubated with either DIR-Exon, DIR-Intron or DIR. Notably, DIR-Intron and DIR reduced the spontaneous excitatory postsynaptic current (sEPSC) amplitude and frequency, whereas neither the amplitude nor the frequency of sEPSCs was altered after DIR-Exon incubation (FIG. 15C-15E; FIG. 19AC-19D). Moreover, long-term potentiation (LTP) of field excitatory postsynaptic potentials (fEPSPs) recorded in the hippocampal CA1 region was apparently decreased in the brain slices from homozygous DIR-KI mice compared with those from WT mice, indicating that DIR suppresses the major excitatory synaptic pathway in the hippocampus (FIG. 15F). Furthermore, we found that local delivery of the peptide DIR-Intron (200 ng per mouse) via osmotic pumps connected to catheters inserted into the CA1 region of the hippocampus decreased novel object recognition (FIG. 15G). Overall, the homozygous DIR-KI mice displayed progressive Aβ deposition as well as increased Tau phosphorylation coinciding with the decline in cognition and memory because of the inhibitory effect of DIR on excitatory synaptic transmission in hippocampal neural circuits.

### Example 12 Alleviating AD-Like Pathological Features with DIR mAb

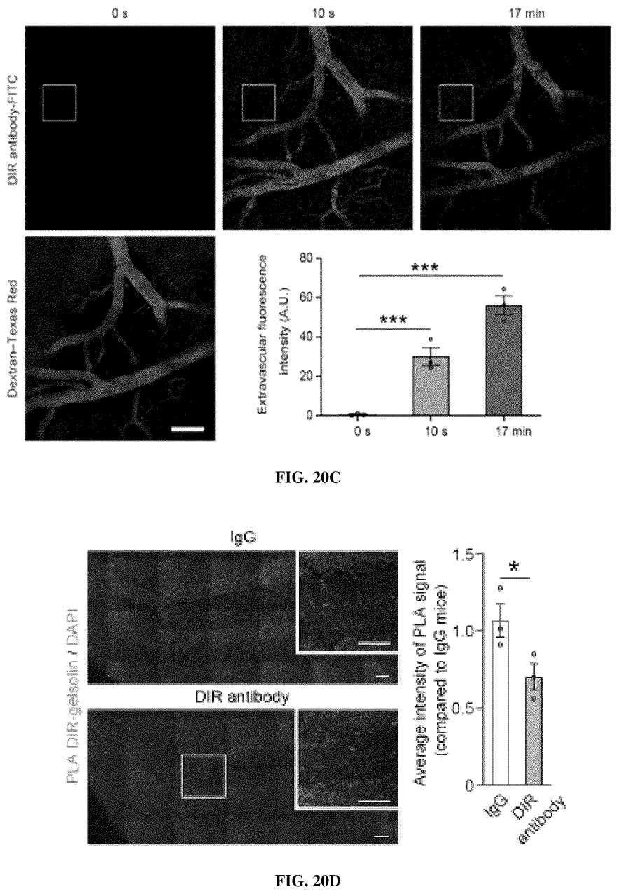

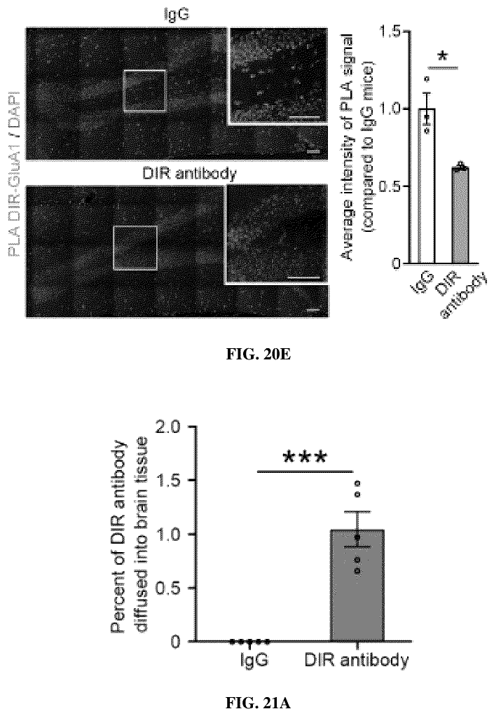

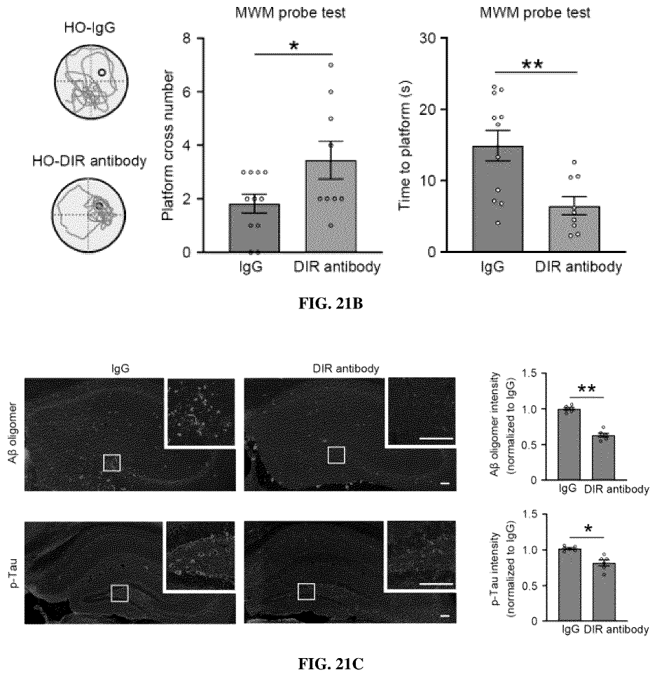

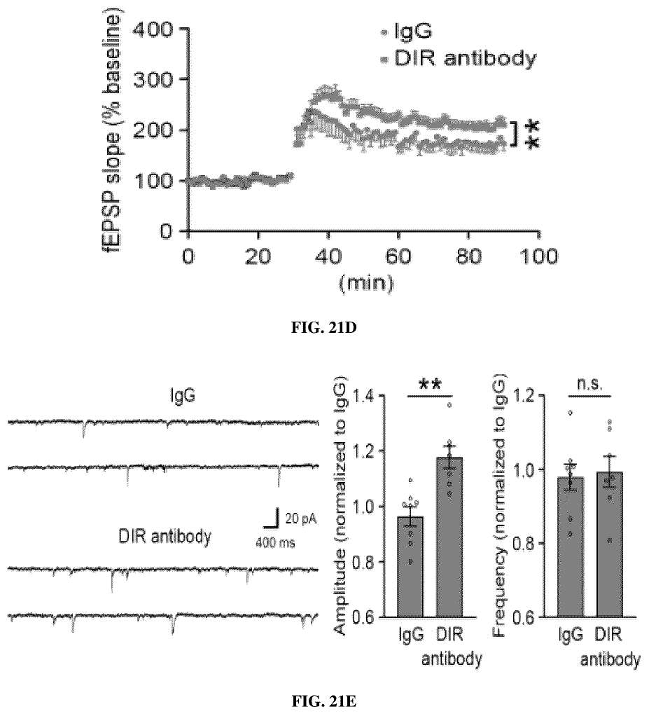

To examine whether a mAb against DIR could alleviate AD-like pathogenesis, we intraperitoneally injected the DIR mAb (0.5 mg/day) or IgG into homozygous DIR-KI mice for 5 days. Both working memory and novel object recognition were improved by mAb treatment (FIGS. 20A-20B). The intraperitoneally injected mAb could be detected in brain tissues by ELISA (FIG. 21A), and DIR mAb-FITC could be diffused in the brain tissue (FIG. 20C), suggesting that the DIR mAb crossed the BBB into brain tissue, and decreased interaction between DIR and GluA1 or DIR and gelsolin in the hippocampi of homozygous DIR-KI mice (FIGS. 20D-20E). In the Morris water maze test, the mice treated with the mAb had a shorter latency to enter the target quadrant and crossed the platform more times (FIG. 21B). Moreover, DIR mAb treatment reduced the expression of Aβ and p-Tau (FIG. 21C). The decrease in hippocampal LTP in the homozygous DIR-KI mice could be rescued by intraperitoneal injection of the mAb (0.5 mg/day, 5 days) (FIG. 21D). Hippocampal slices from DIR-KI mice intraperitoneally injected with the DIR mAb showed increased sEPSC amplitude, compared with those from DIR-KI mice intraperitoneal injected with IgG (FIG. 21E). Collectively, these results indicate that inhibiting DIR activity with the DIR mAb not only decreased the levels of Aβ and p-Tau but also reversed the cognition and memory deficits in homozygous DIR-KI mice, indicating the important role of DIR in AD-like pathogenesis.

All the documents mentioned in the present application are hereby incorporated by reference in their entireties, just as each document is cited separately as a reference. In addition, it should be understood that various modifications or changes can be made by those skilled in the art after reading the above teachings of the present application, and these equivalent forms shall also fall within the scope defined by the claims appended hereto.

What is claimed is:

1. A method for diagnosing cognitive impairment or a neurodegenerative disease, and/or evaluating progression of said cognitive impairment or said neurodegenerative disease, comprising the step of: detecting, from a sample of a subject in need thereof, a content of a DNA damage-inducible transcript 4-like DDIT4L intron retention spliced product DIR, and/or of a nucleic acid encoding said DIR.

2. The method according to claim 1, wherein said DIR comprises the amino acid sequence as set forth in SEQ ID NO: 1.

3. The method according to claim 1, wherein said cognitive impairment comprises cognitive impairment caused by normal aging, Lewy body dementia (LBD), frontotemporal dementia, vascular dementia, and/or cognitive impairment induced by a disease, wherein the cognitive impairment-inducing disease comprises Alzheimer's disease, multi-infarct type, Parkinson's disease, AIDS and/or Creutzfeldt-Jakob disease (CJD).

4. The method according to claim 1, wherein said cognitive impairment comprises mild cognitive impairment MCI, intermediate cognitive impairment and late cognitive impairment.

5. The method according to claim 1, wherein said cognitive impairment comprises multi-domain amnestic MCI (aMCI-m).

6. The method according to claim 1, wherein said neurodegenerative disease comprises a neurodegenerative disease caused by neuronal death and glial cell homeostasis, a neurodegenerative disease caused by aging, a neurodegenerative disease caused by affected CNS cell function, a neurodegenerative disease caused by abnormal intercellular communication and/or a neurodegenerative disease caused by impaired cell mobility.

7. The method according to claim 1, wherein said neurodegenerative disease comprises Alzheimer's disease, Parkinson's disease, multiple sclerosis MS, amyotrophic lateral sclerosis (ALS) Huntington's disease (HD), and/or presenile dementia.

8. The method according to claim 1, wherein said sample comprises a cerebrospinal fluid sample, a blood sample a tissue and/or a cell sample, wherein said blood sample comprises whole blood, serum, and/or plasma.

9. The method according to claim 1, wherein said method comprises: comparing the content of said DIR and/or of said nucleic acid encoding said DIR in the sample of said subject with a control value, wherein the higher content of said DIR and/or of said nucleic acid encoding said DIR in the sample of said subject relative to the control value indicates said subject as having said cognitive impairment and/or said neurodegenerative disease, or the progression of the cognitive impairment and/or neurodegenerative disease afflicting the subject has worsened.

10. The method according to claim 9, wherein said control value comprises a normal control value or an early control value, wherein said normal control value comprises a content of said DIR and/or of said nucleic acid encoding said DIR in a normal subject, wherein said early control value comprises a previously measured content of said DIR and/or of said nucleic acid encoding said DIR in said same subject.

11. The method according to any one of claim 1, further comprising the step of detecting a content of a marker associated with said cognitive impairment and/or said neurodegenerative disease in a sample derived from said subject, and/or the step of observing brain imaging of said subject.

12. The method according to claim 11, wherein said marker associated with said cognitive impairment and/or said neurodegenerative disease comprises AD7C-NTP, pTau-181, pTau-217, Aβ40 and/or Aβ42.

13. A diagnostic kit, comprising a substance for detecting a DNA damage-inducible transcript 4-like DDIT4L intron retention spliced product DIR, and/or a nucleic acid encoding said DIR, wherein said diagnostic kit is for use in diagnosis of cognitive impairment, and/or evaluation of progression of said cognitive impairment; and/or for use in diagnosis of a neurodegenerative disease, and/or evaluation of progression of said neurodegenerative disease.

14. The diagnostic kit according to claim 13, wherein said DIR comprises the amino acid sequence as set forth in SEQ ID NO: 1.

15. The diagnostic kit according to claim 13, wherein said substance comprises a reagent and/or an apparatus capable of detecting said DIR, and/or said nucleic acid encoding said DIR.

16. The diagnostic kit according to claim 13, wherein said substance comprises an antigen-binding protein capable of specifically binding to said DIR, a probe capable of specifically binding to said DIR, a primer capable of specifically amplifying said nucleic acid encoding said DIR, a gene chip capable of specifically detecting said nucleic acid encoding said DIR, an aptamer capable of specifically binding to said nucleic acid encoding said DIR, and/or a guide RNA capable of specifically binding to said nucleic acid encoding said DIR.

17. The diagnostic kit according to claim 16, wherein said antigen-binding protein capable of specifically binding to said DIR comprises HCDR3, HCDR2, HCDR1, LCDR3, LCDR2 and LCDR1, wherein:
a) the HCDR1 comprises the amino acid sequence as set forth in SEQ ID NO: 20, the HCDR2 comprises the amino acid sequence as set forth in SEQ ID NO: 21, the HCDR3 comprises the amino acid sequence as set forth in SEQ ID NO: 22, the LCDR1 comprises the amino acid sequence as set forth in SEQ ID NO: 25, the LCDR2 comprises the amino acid sequence as set forth in SEQ ID NO: 26, the LCDR3 comprise the amino acid sequence as set forth in SEQ ID NO: 27;
b) the HCDR1 comprises the amino acid sequence as set forth in SEQ ID NO: 20, the HCDR2 comprises the amino acid sequence as set forth in SEQ ID NO: 21, the HCDR3 comprises the amino acid sequence as set forth in SEQ ID NO: 30, the LCDR1 comprises the amino acid sequence as set forth in SEQ ID NO: 25, the LCDR2 comprises the amino acid sequence as set forth in SEQ ID NO: 26, the LCDR3 comprises the amino acid sequence as set forth in SEQ ID NO: 27,
c) the HCDR1 comprises the amino acid sequence as set forth in SEQ ID NO: 35, the HCDR2 comprises the amino acid sequence as set forth in SEQ ID NO: 36, the HCDR3 comprises the amino acid sequence as set forth in SEQ ID NO: 37, the LCDR1 comprises the amino acid sequence as set forth in SEQ ID NO: 25, the LCDR2 comprises the amino acid sequence as set forth in SEQ ID NO: 26, the LCDR3 comprises the amino acid sequence as set forth in SEQ ID NO: 27,
d) the HCDR1 comprises the amino acid sequence as set forth in SEQ ID NO: 20, the HCDR2 comprises the amino acid sequence as set forth in SEQ ID NO: 42, the HCDR3 comprises the amino acid sequence as set forth in SEQ ID NO: 43, the LCDR1 comprises the amino acid sequence as set forth in SEQ ID NO: 25, the LCDR2 comprises the amino acid sequence as set forth in SEQ ID NO: 26, the LCDR3 comprises the amino acid sequence as set forth in SEQ ID NO: 27,
e) the HCDR1 comprises the amino acid sequence as set forth in SEQ ID NO: 20, the HCDR2 comprises the amino acid sequence as set forth in SEQ ID NO: 48, the HCDR3 comprises the amino acid sequence as set forth in SEQ ID NO: 49, the LCDR1 comprises the amino acid sequence as set forth in SEQ ID NO: 52, the LCDR2 comprises the amino acid sequence as set forth in SEQ ID NO: 26, the LCDR3 comprises the amino acid sequence as set forth in SEQ ID NO: 53,
f) the HCDR1 comprises the amino acid sequence as set forth in SEQ ID NO: 56, the HCDR2 comprises the amino acid sequence as set forth in SEQ ID NO: 36, the HCDR3 comprises the amino acid sequence as set forth in SEQ ID NO: 57, the LCDR1 comprises the amino acid sequence as set forth in SEQ ID NO: 25, the LCDR2 comprises the amino acid sequence as set forth in SEQ ID NO: 26, the LCDR3 comprises the amino acid sequence as set forth in SEQ ID NO: 27,
g) the HCDR1 comprises the amino acid sequence as set forth in SEQ ID NO: 35, the HCDR2 comprises the amino acid sequence as set forth in SEQ ID NO: 36, the HCDR3 comprises the amino acid sequence as set forth in SEQ ID NO: 62, the LCDR1 comprises the amino acid sequence as set forth in SEQ ID NO: 25, the LCDR2 comprises the amino acid sequence as set forth in SEQ ID NO: 26, the LCDR3 comprises the amino acid sequence as set forth in SEQ ID NO: 27,
h) the HCDR1 comprises the amino acid sequence as set forth in SEQ ID NO: 35, the HCDR2 comprises the amino acid sequence as set forth in SEQ ID NO: 42, the HCDR3 comprises the amino acid sequence as set forth in SEQ ID NO: 37, the LCDR1 comprises the amino acid sequence as set forth in SEQ ID NO: 52, the LCDR2 comprises the amino acid sequence as set forth in SEQ ID NO: 26, the LCDR3 comprises the amino acid sequence as set forth in SEQ ID NO: 53,
i) the HCDR1 comprises the amino acid sequence as set forth in SEQ ID NO: 56, the HCDR2 comprises the amino acid sequence as set forth in SEQ ID NO: 36, the HCDR3 comprises the amino acid sequence as set forth in SEQ ID NO: 57, the LCDR1 comprises the amino acid sequence as set forth in SEQ ID NO: 52, the LCDR2 comprises the amino acid sequence as set forth in SEQ ID NO: 26, the LCDR3 comprises the amino acid sequence as set forth in SEQ ID NO: 67,
j) the HCDR1 comprises the amino acid sequence as set forth in SEQ ID NO: 10, the HCDR2 comprises the amino acid sequence as set forth in SEQ ID NO: 11, the HCDR3 comprises the amino acid sequence as set forth in SEQ ID NO: 12, the LCDR1 comprises the amino acid sequence as set forth in SEQ ID NO: 15, the LCDR2 may contain the amino acid sequence as set forth in SEQ ID NO: 16, and the LCDR3 comprises the amino acid sequence as set forth in SEQ ID NO: 17.

18. The diagnostic kit according to claim 16, wherein said antigen-binding protein comprises a heavy chain variable region VH and a light chain variable region VL, wherein:
a) said VH comprises the amino acid sequence as set forth in SEQ ID NO: 23 and said VL comprises the amino acid sequence as set forth in SEQ ID NO: 28;
b) said VH comprises the amino acid sequence as set forth in SEQ ID NO: 31 and said VL comprises the amino acid sequence as set forth in SEQ ID NO: 33;
c) said VH comprises the amino acid sequence as set forth in SEQ ID NO: 38 and said VL comprises the amino acid sequence as set forth in SEQ ID NO: 40;
d) said VH comprises the amino acid sequence as set forth in SEQ ID NO: 44 and said VL comprises the amino acid sequence as set forth in SEQ ID NO: 46;
e) said VH comprises the amino acid sequence as set forth in SEQ ID NO: 50 and said VL comprises the amino acid sequence as set forth in SEQ ID NO: 54;
f) said VH comprises the amino acid sequence as set forth in SEQ ID NO: 58 and said VL comprises the amino acid sequence as set forth in SEQ ID NO: 60;
g) said VH comprises the amino acid sequence as set forth in SEQ ID NO: 63 and said VL comprises the amino acid sequence as set forth in SEQ ID NO: 60;
h) said VH comprises the amino acid sequence as set forth in SEQ ID NO: 65 and said VL comprises the amino acid sequence as set forth in SEQ ID NO: 54;
i) said VH comprises the amino acid sequence as set forth in SEQ ID NO: 58 and said VL comprises the amino acid sequence as set forth in SEQ ID NO: 68; or
j) said VH comprises the amino acid sequence as set forth in SEQ ID NO: 13, and said VL comprises the amino acid sequence as set forth in SEQ ID NO: 18.

19. The diagnostic kit according to claim 16, wherein said antigen-binding protein comprises a heavy chain constant region, which comprises an IgG-derived constant region.

20. A medicament screening method, comprising the step of: detecting changes in a content of DIR and/or of a nucleic acid encoding said DIR in a subject after administration of a candidate medicament, wherein said medicament is for use in prevention, treatment and/or alleviation of cognitive impairment and/or a neurodegenerative disease.
